(19) United States
(12) Patent Application Publication
(10) Pub. No.: US 2025/0131980 A1
(43) Pub. Date: Apr. 24, 2025

(54) SYSTEMS AND METHODS FOR DISCOVERY AND PREDICTING PHENOTYPES

(71) Applicant: THE TEXAS A&M UNIVERSITY SYSTEM, College Station, TX (US)

(72) Inventors: Seth C. Murray, College Station, TX (US); Alper Adak, College Station, TX (US); Steven Anderson, College Station, TX (US); Aaron DeSalvio, College Station, TX (US); Holly Lane, College Station, TX (US); Shakirah Nakasagga, College Station, TX (US)

(21) Appl. No.: 18/921,951

(22) Filed: Oct. 21, 2024

Related U.S. Application Data
(60) Provisional application No. 63/591,561, filed on Oct. 19, 2023.

(51) Int. Cl. G16B 20/00; G16B 40/20

(52) U.S. Cl. CPC G16B 20/00; G16B 40/20

(57) ABSTRACT

[symbol]

An example method of predicting a phenotype of interest of a subject includes generating apluralityofbiomarkers; selectingasubsetofbiomarkersfromthepluralityofbiomarkers;anddeterminin garelationshipbetweenthesubsetofbiomarkersandthephenotypeofinterest;receivingsample biomarkers;where thesample biomarkerscomprise oneor more measurementsofthesubject;andpredictingthephenotypeofinterestofthesubjectbasedonthesampleb iomarkers and the relationship between the subsetof biomarkers and the phenotype of interest.

[symbol] SYSTEMS AND METHODS FOR DISCOVERY AND PREDICTING PHENOTYPES

### CROSS REFERENCE TO RELATED APPLICATIONS

Thisapplicationclaimspriority to, and the benefitof, U.S.Provisional PatentApplication No. 63/591,561, filed Oct. 19, 2023, which is incorporated by reference herein in itsentirety.

STATEMENT REGARDINGFEDERALLY FUNDED RESEARCH

Thisinventionwasmadewithgovernmentsupportunder2020-68013- 32371awardedbytheNationalInstituteofFoodandAgriculture,2021-67013- 33915awardedbytheNational Institute of Food and Agriculture, and TEX0-2-9348 awarded by the National Institute ofFoodandAgriculture. Thegovernmenthascertainrightsintheinvention.

### BACKGROUND

Aphenotypeisanobservablecharacteristicofanorganism (e.g., aplantoranimal) offun ctional,biologicaloreconomicinterest.Phenotypescanincludeawiderangeoffeatures,includingphysic alattributes, behavioraltraits, andphysiologicalfunctions (e.g., metabolism, disease resistance). Phenotypes are the observable endpoints of complex interactionsbetweenanorganismsgenesandtheenvironment. Thus, observing the phenotypeis anefficientwaytoselectorganismforbreeding,predictthebehaviororperformanceoforganisms, andot herwiseunderstandtheorganism.Phenotypesarefrequentlyobservedandusedforbreedingandpredict ion,evenwhentheorganism′sgenotypeisnotcompletelyknownorunderstood.

However,a potentially infinite number of phenotypesare possible for any given organism andincombinations unique to that individual. Improvements to determining and predicting phenotypescanimprovethepredictionofperformanceoforganisms,suggestmanagementorhealthinter ventions, aswellastheselectionoforganismsforbreedingorgeneticimprovement.

### SUMMARY

In some aspects, the techniques described herein relate to a method of selectingbiomarkers, themethodincluding: generatingapluralityofbiomarkers;selectingasubsetof biomarkers from the plurality of biomarkers; and determining a relationship between the subset ofbiomarkersandoneormorephenotypesofinterest.

In some aspects, the techniques described herein relate to a method, whereinselecting the subset of biomarkers includes selecting heritable biomarkers from the plurality ofbiomarkers.

In some aspects, the techniques described herein relate to a method, wherein thepluralityofbiomarkersincludesatleastoneofgenetic data, metabolomic dataorproteomic data.

In some aspects, the techniques described herein relate to a method, wherein thepluralityofbiomarkersaregeneratedusingaconvolutionalneuralnetwork.

Insomeaspects, thetechniquesdescribedhereinrelatetoamethod, whereinselectin gthesubsetofbiomarkersincludesselectingbiomarkersbasedacorrelationbetweenabiomarker of the plurality of biomarkers with a different biomarker of the plurality of biomarkers.

In some aspects, the techniques described herein relate to a method, whereindeterminingarelationshipbetweenthesubsetofbiomarkersandthephenotypeofinterestis basedonamachinelearningtestofpredictionability.

In some aspects, the techniques described herein relate to a method, wherein themachinelearning testofpredictionabilityincludesalassotest, aridgeregressiontest,aBayesB . . . orarandomforestregression test.

In some aspects, the techniques described herein relate to a method, wherein themachinelearningtestfurtherincludescrossvalidation.

In some aspects, the techniques described herein relate to a method, wherein thepluralityofbiomarkersincluderemotelysenseddata.

In some aspects, the techniques described herein relate to a method, wherein thepluralityofbiomarkersincludetemporally (longitudinally) measuredbiomarkers.

In some aspects, the techniques described herein relate to a method, wherein themethodfurtherincludesdetermininganoptimalmanagementstrategyforimprovinghealth,producti onorothervalueaddedtraitforasubject, whereintheoptimalmanagementstrategyforthesubjectisconfi guredtochangethephenotypeofinterestofthesubject.

Insomeaspects, thetechniquesdescribedhereinrelatetoamethod, wherein the method further includes predicting a chance of success of the phenotype of interest in a selectivebreedingprogram.

In some aspects, the techniques described herein relate to a method, whereinphenotypeofinterestincludesadiseaseorriskofdisease.

In some aspects, the techniques described herein relate to a method, wherein thephenotypeincludesaplantphenotype.

In some aspects, the techniques described herein relate to a method, wherein theplantphenotypeincludesanestimateofplantheightorplantyield.

In some aspects, the techniques described herein relate to a method, wherein thepluralityofbiomarkersincludeimagedata.

Insomeaspects, thetechniquesdescribedhereinrelatetoamethod, furtherincluding decomposing the image data into a plurality of image features, which can be used as onetypeofbiomarker.

In some aspects, the techniques described herein relate to a method, wherein thepluralityofimagefeaturesincludeestimatesofapositionororientationofasubject.

In some aspects, the techniques described herein relate to a method, wherein thepluralityofimagefeaturesincludeestimatesofasizeofasubject.

In some aspects, the techniques described herein relate to a method of predictingaphenotypeofinterestofasubject, themethodincluding: generatingapluralityofbiomarkers;s electingasubsetofbiomarkersfromthepluralityofbiomarkers;anddeterminingarelationshipbetweent hesubsetofbiomarkersandthephenotypeofinterest;receivingsamplebiomarkers;whereinthesampleb iomarkersincludeoneormoremeasurementsofthesubject;andpredictingthephenotypeofinterestofth esubjectbasedonthesamplebiomarkers andtherelationshipbetweenthesubsetofbiomarkersandthephenotypeofinterest.

In some aspects, the techniques described herein relate to a method, whereinselectingthesubsetofbiomarkersincludesselectingheritableorotherwiserepeatableandin heritedbiomarkersfromthepluralityofbiomarkers.

In some aspects, the techniques described herein relate to a method, wherein thepluralityofbiomarkersincludesatleastoneofgenetic data, metabolomic dataorproteomic data.

Insomeaspects, thetechniquesdescribedhereinrelatetoamethod, wherein the plurality of biomarkers are generated using a neural network.

Insomeaspects, thetechniquesdescribedhereinrelatetoamethod, whereinselectin gthesubsetofbiomarkersincludesselectingbiomarkersbasedacorrelationbetweenabiomarker of the plurality of biomarkers with a different biomarker of the plurality of biomarkers.

In some aspects, the techniques described herein relate to a method, whereindeterminingarelationshipbetweenthesubsetofbiomarkersandthephenotypeofinterestis basedonamachinelearningtestofpredictionability.

In some aspects, the techniques described herein relate to a method, wherein themachine learning test of prediction ability includes a lasso test, a ridge regression test, or a randomforestregressiontest.

In some aspects, the techniques described herein relate to a method, wherein themachinelearningtestfurtherincludescrossvalidation.

In some aspects, the techniques described herein relate to a method, wherein thepluralityofbiomarkersincluderemotelysenseddata.

In some aspects, the techniques described herein relate to a method, wherein thepluralityofbiomarkersincludetemporallymeasuredbiomarkers.

In some aspects, the techniques described herein relate to a method, wherein themethod further includes determining an optimal management strategy for the subject, wherein theoptimalmanagementstrategyforthesubjectisconfiguredtochangethe phenotypeofinterestofthesubject.

In some aspects, the techniques described herein relate to a method, wherein themethodfurtherincludespredictingachanceofsuccessofthesubjectinaselectivebreedingprogram.

In some aspects, the techniques described herein relate to a method, whereinphenotypeofinterestofthesubjectincludesadiseaseorriskofdisease.

In some aspects, the techniques described herein relate to a method of predictingaphenotypeofinterestofasubject, themethodincluding: determininga compositescoreofthesubject, whereinthecompositescoreisbasedonaplurality ofmeasurements ofthesubject;and predicting a phenotype of interest of the subject based on the composite score of the subject and abiomarkermodel.

In some aspects, the techniques described herein relate to a method, wherein thebiomarkermodelincludesbiomarkersselectedbasedonacorrelationbetweenabiomarkerofapluralit yofbiomarkerswithadifferentbiomarkerofthepluralityofbiomarkers.

In some aspects, the techniques described herein relate to a method, wherein thepluralityofmeasurementsofthesubjectaretemporally (longitudinally) separated.

In some aspects, the techniques described herein relate to a method, wherein thepluralityofbiomarkersincludeimagedata.

In some aspects, the techniques described herein relate to a method, furtherincludingdecomposingtheimagedataintoapluralityofimagefeatures.

In some aspects, the techniques described herein relate to a method, wherein thepluralityofimagefeaturesincludeestimatesofapositionororientationofasubject.

In some aspects, the techniques described herein relate to a method, wherein thepluralityofimagefeaturesincludeestimatesofasizeofasubject.

In some aspects, the techniques described herein relate to a method, wherein thephenotypeincludesaplantphenotype.

In some aspects, the techniques described herein relate to a method, wherein theplantphenotypeincludesanestimateofplantheightorplantyield.

Insomeaspects, thetechniquesdescribedhereinrelatetoasystemincluding: aremote sensing platform including a remote sensor configured to capture a plurality of biomarkers, whereinthebiomarkersincludeimagedata;acomputingdeviceoperablycoupledtotherem otesensingplatform, whereinthecomputingdeviceincludesatleastoneprocessorandmemory, themem oryhavingcomputer-executableinstructionsstoredthereonthat, whenexecutedbytheatleast one processor, cause the at least one processor to: selecting a subset of biomarkers from thepluralityofbiomarkers;anddeterminingarelationshipbetweenthesubsetofbiomarkersandaphenot ypeofinterest.

In some aspects, the techniques described herein relate to a system, wherein theremotesensingplatformincludesanunmannedaerialvehicle (alsotermedunoccupiedaerialsystem, droneorsimilar) orroboticcollectionsystem.

In some aspects, the techniques described herein relate to a system, wherein theremotesensingplatformisconfiguredtoimagecrops.

In some aspects, the techniques described herein relate to a system, wherein theremotesensingplatformisconfiguredtoacquireapluralityoftemporally-spacedimages.

In some aspects, the techniques described herein relate to a system, wherein thememory has further computer-executable instructions stored thereon that, when executed by theatleastoneprocessor, causetheatleastoneprocessorto,causetheremotesensing platformtocontrol theremotesensingplatform basedontherelationshipbetweenthesubsetofbiomarkersandthephenotypeofinterest.

Itshouldbeunderstoodthattheabove- describedsubjectmattermayalsobeimplemented as a computer-controlled apparatus, a computer process, a computing system, or anarticleofmanufacture,suchasacomputer- readablestoragemedium.

Othersystems, methods, featuresand/oradvantageswillbeormaybecomeapparentt oonewithskillintheartuponexaminationofthefollowingdrawingsanddetaileddescription. It is intended that all such additional systems, methods, features and/or advantages beincludedwithinthisdescriptionandbeprotectedbytheaccompanyingclaims.

### BRIEF DESCRIPTION OF THE DRAWINGS

The components in the drawings are not necessarily to scale relative to eachother.Likereferencenumeralsdesignatecorrespondingpartsthroughouttheseveralviews. FIG. 1Aillustratesanexamplemethodofdeterminingarelationshipbetweenbiomar kers and a phenotype of interest, according to implementations of the present disclosure.

FIG. 1B illustrates an example method of predicting a phenotype of interest of asubject based on a subset of biomarkers, according to implementations of the present disclosure.

FIG. 1Cillustratesanexamplemethodofpredictingaphenotypeusingacomposite score and biomarker model, according to implementations of the present disclosure.

FIG. 2 illustrates an example system for predicting phenotypes using remotesensing,accordingtoimplementationsofthepresentdisclosure.

FIG. 3 illustrates an example computing device.

FIG. 4 illustrates a flowchart of an example method, according toimplementationsofthepresentdisclosure.

FIG. 5shows a family showing resemblance over time. Siblings look similar andhave facial features suggesting relationships, but exact features and measurements differ due totemporalgeneticsanddivergentinteractionswithenvironments.

FIG. 6showsgenomiccorrelationsandphenomiccorrelationson520recombinant inbred lines. Genomic data had 11,334 SNPs after cleaning. Phenomic data included896features (32RGBderivedvegetationindicesover14flightdatesandtwomanagementenvir onments, drought and irrigated). Both have measures with high and low correlations (+or-) andareunsaturated.

FIGS. 7A-7Ishowagraphicaloverviewofdatacollection, visualandvegetationindex- based senescence scoring of cotton, and preparation of data for CNN training and evaluation.FIG. 7Ashowsorthomosaicsfromarepresentativesampleof3ofthe14flightscapturingthesen escencewindowareshowninsequentialorder;FIG. 7Bshowssingle-plantshapefilesconstructed and overlaid on each orthomosaic with minor adjustments to boundary boxes made asneeded; FIG. 7CshowsindividualGeoTIFFsextractedfromeachorthomosaicandconvertedtoJPEGs;

FIG. 7D shows plants visually scored for senescence training data using images from FIG. 7C;FIG. 7Eshowsempiricalsenescencescoresobtainedbycalculatingvegetationindicesforeachimageas showninFIG.7CusingtheFIELDimageRpackage;FIG. 7Fshowsthatfunctionalprincipalcomponentanalysi s (FPCA) wasusedtoassesstemporaldatafromvisualsenescenceratings (VSRs), RCCindexvalues, andTND GRindexvalues; FIG. 7Gshowsthefirstfunctionalprincipalcomponentvalues, FPC1, wereusedinANOVA;F IG.7Hshowstime- seriesimage (TSI), or “imagesandwich” creationisoutlined;FIG. 7|presentsCNNdatapartitioning,whereo ptimalmodelhyperparameters were determinedusing 50% ofTSIs from experiment1 (E1) andevaluatedonallimages (unseen) fromE2.

FIGS. 8A-

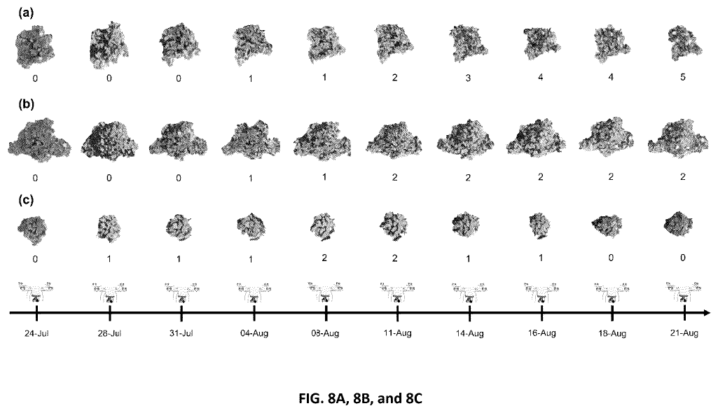

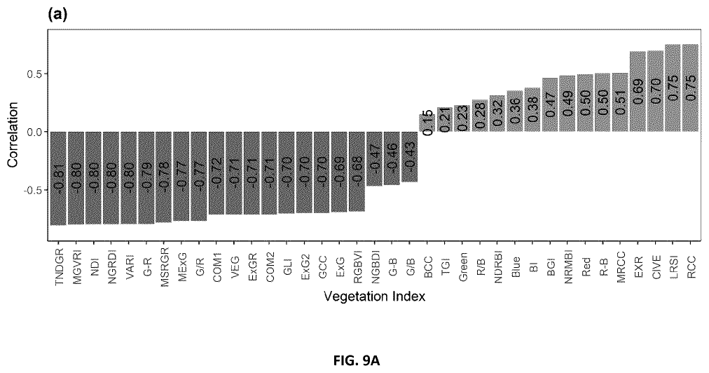

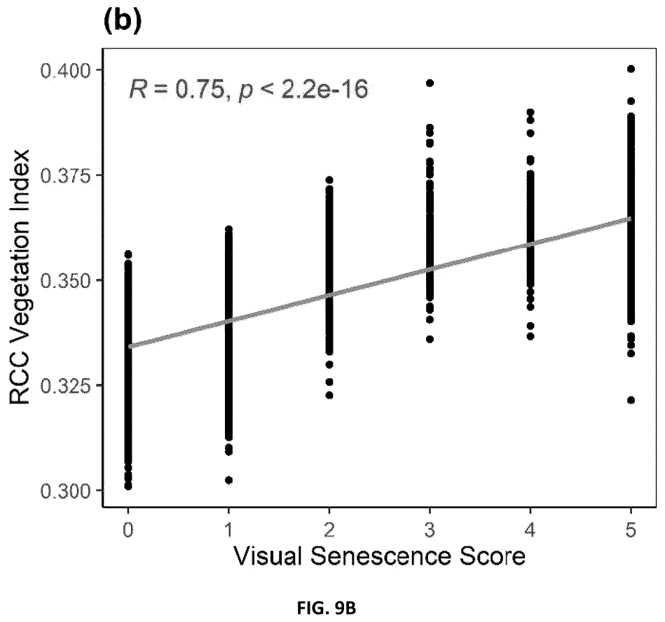

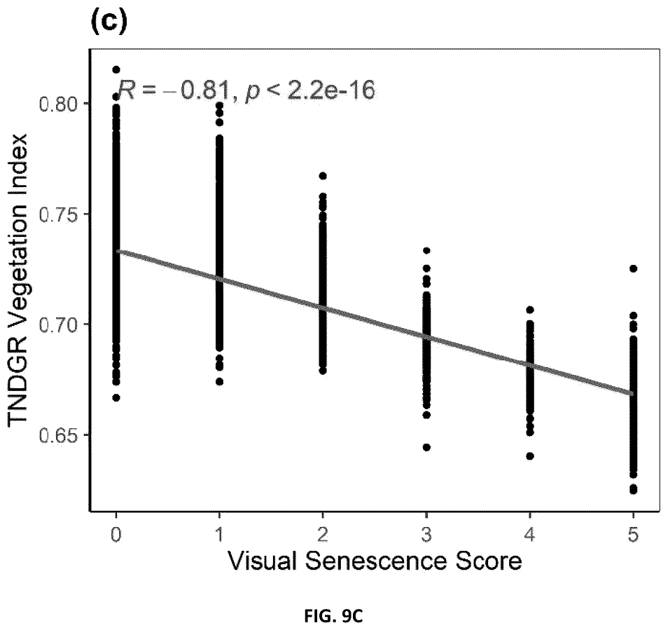

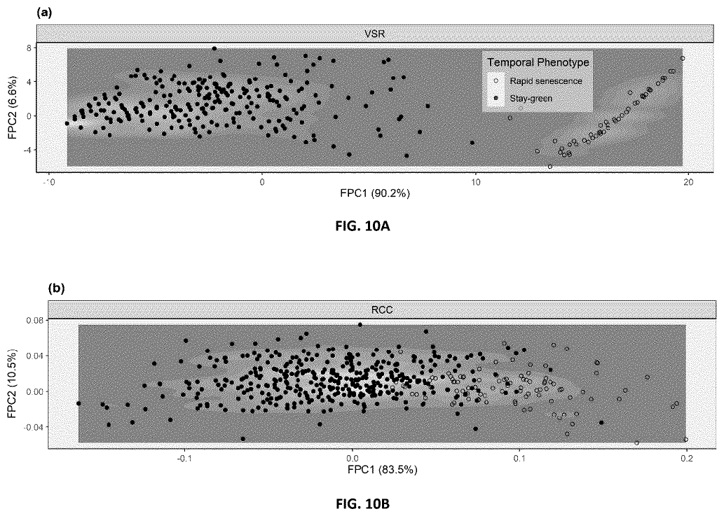

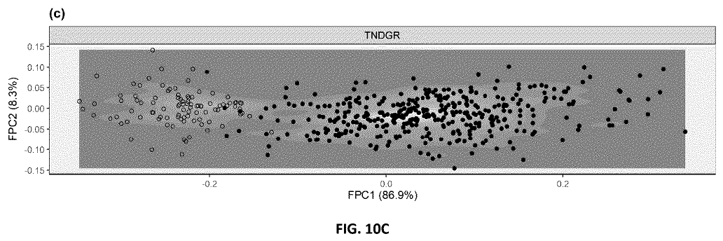

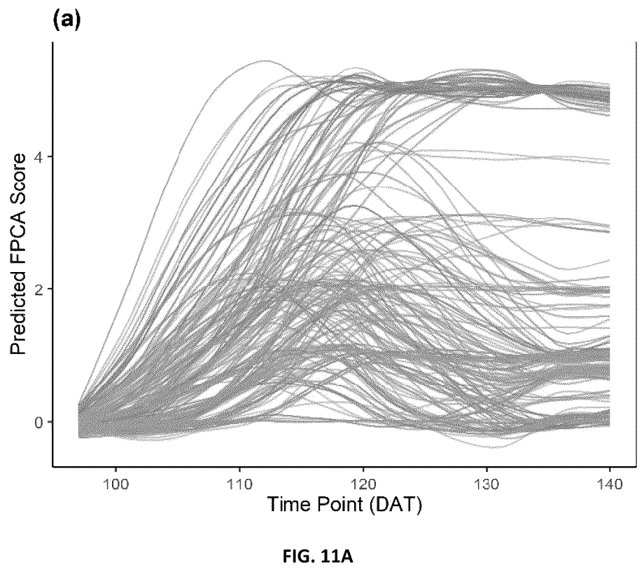

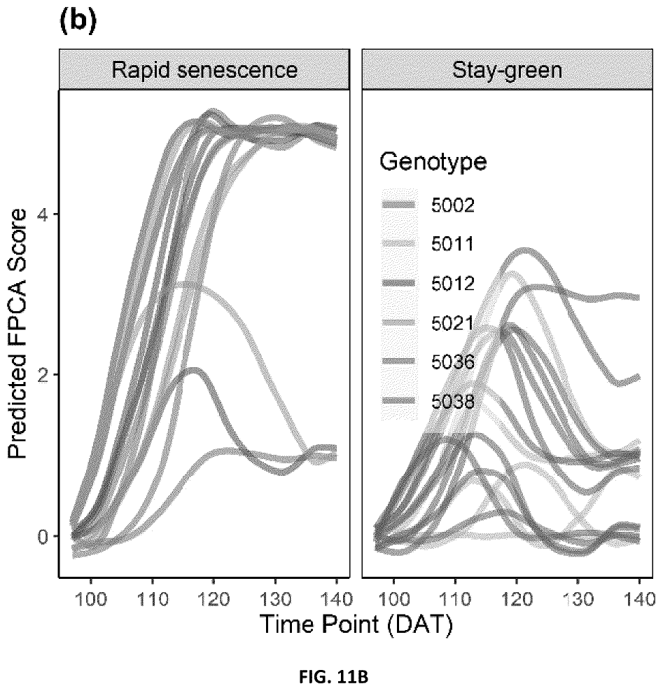

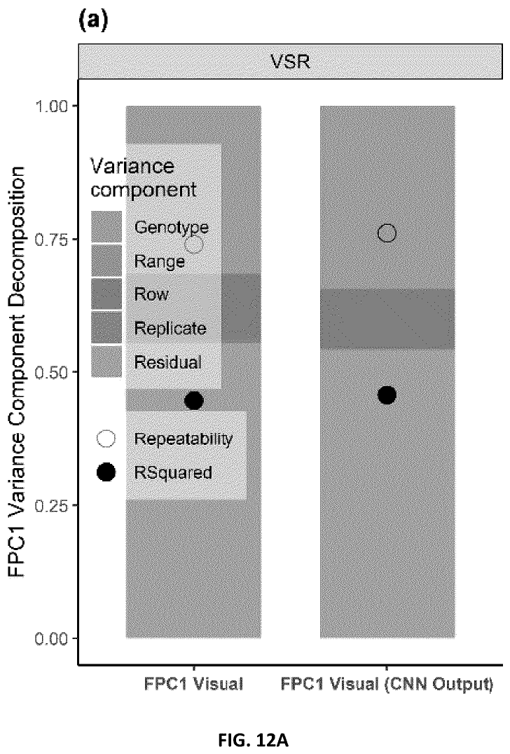

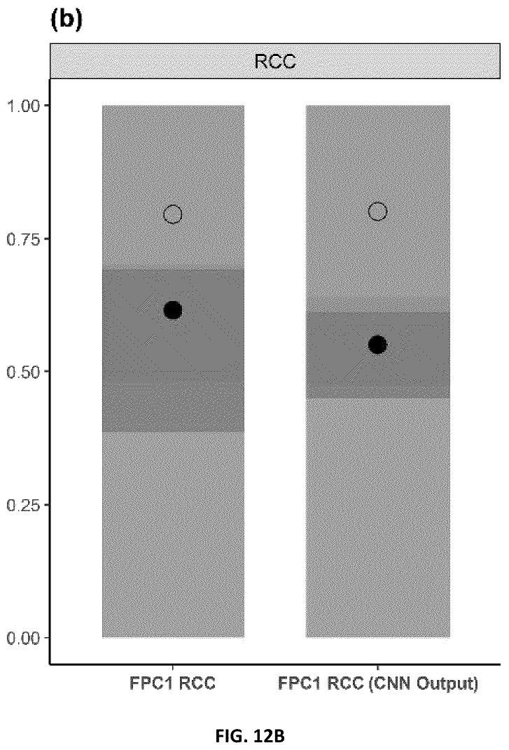

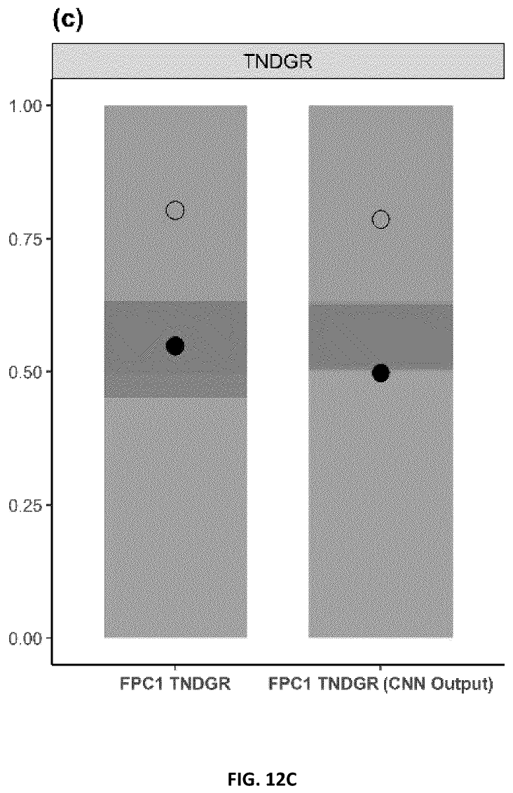

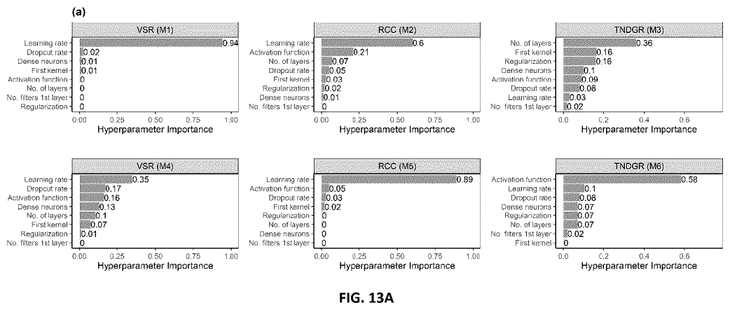

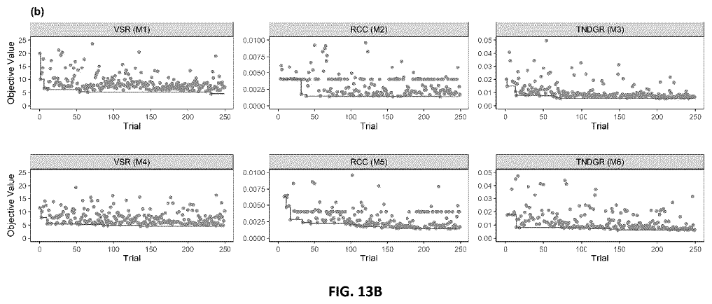

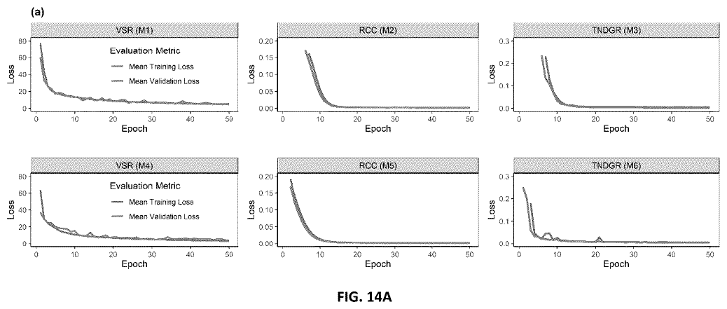

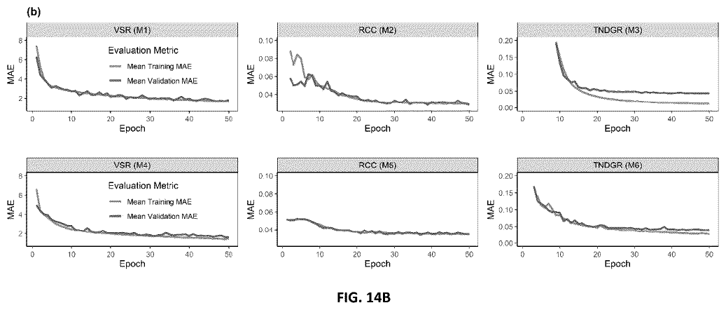

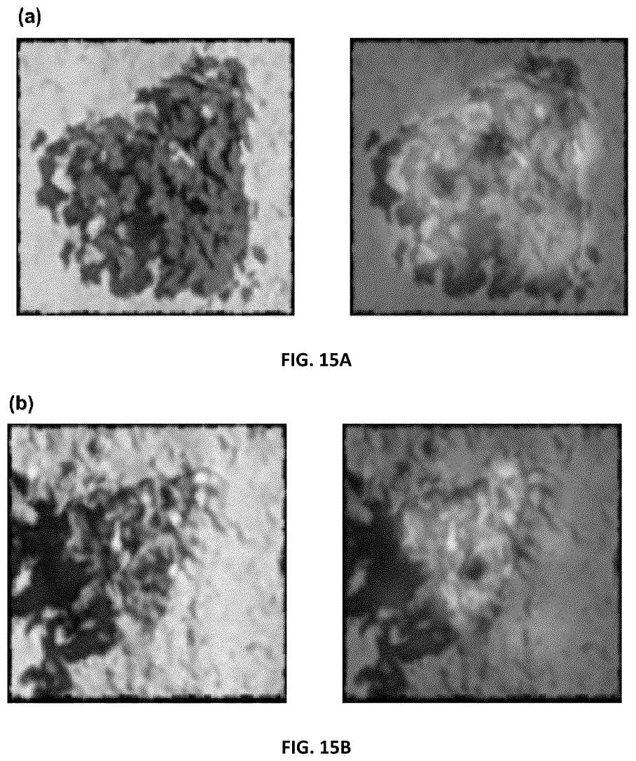

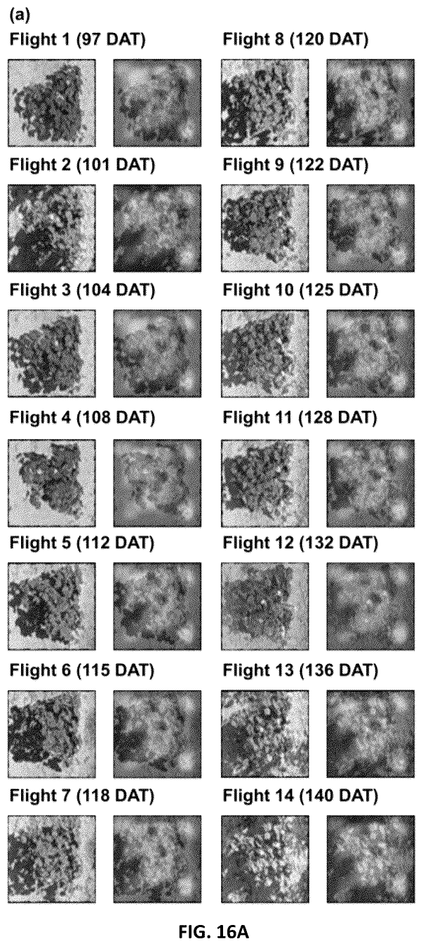

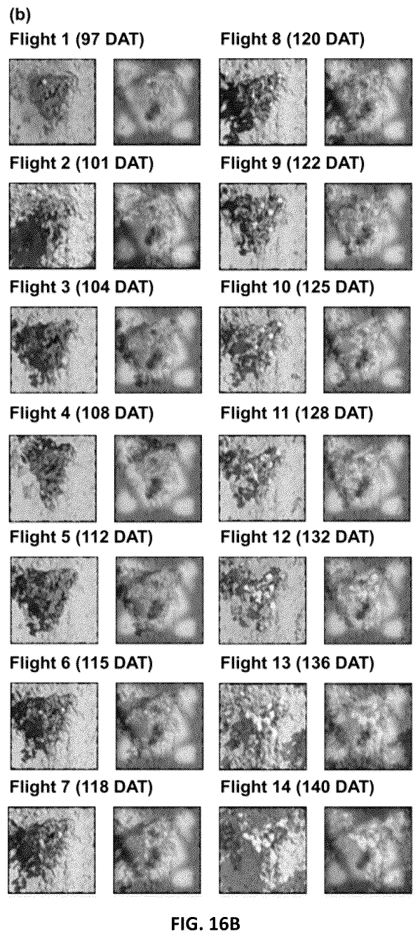

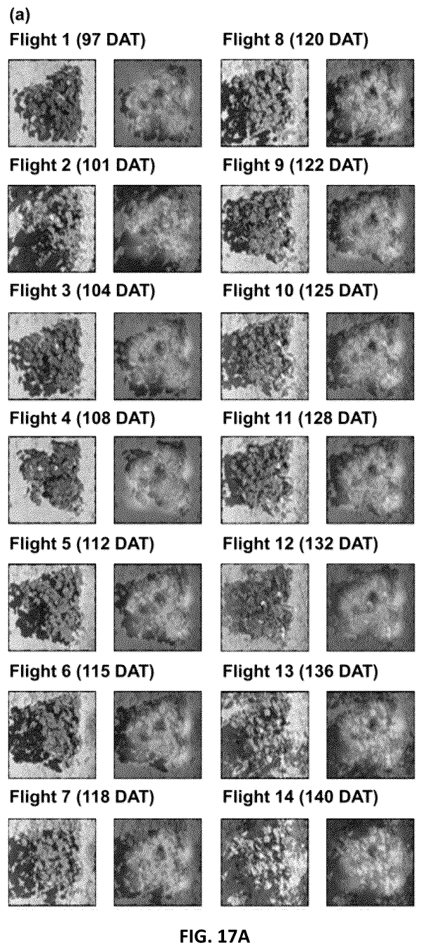

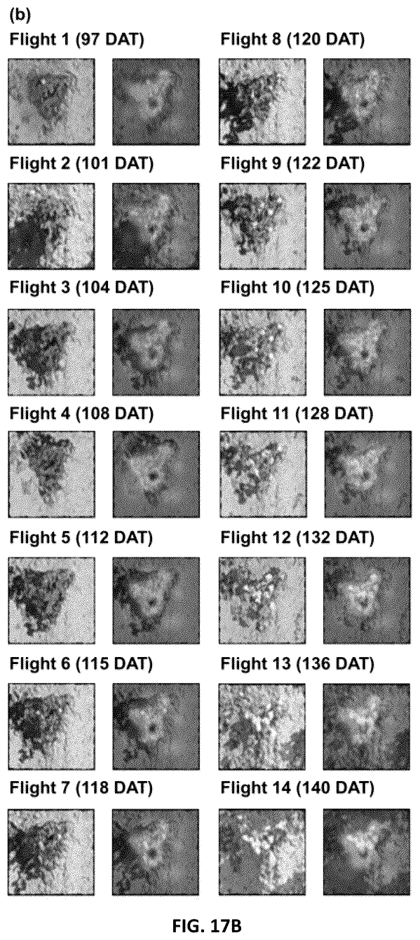

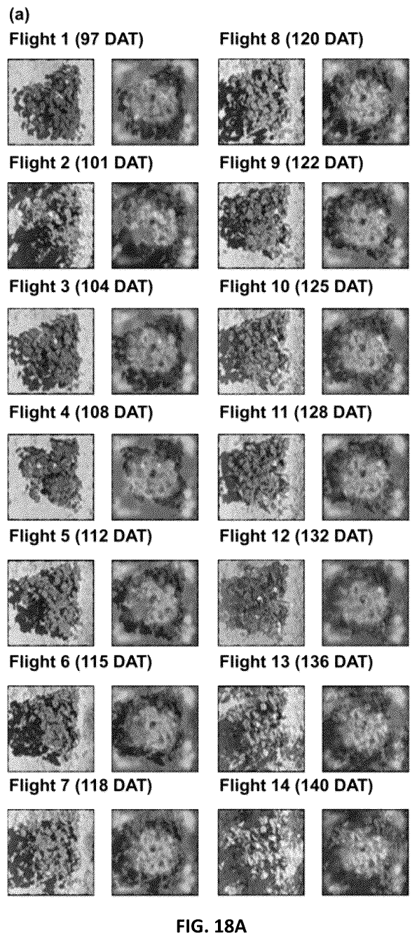

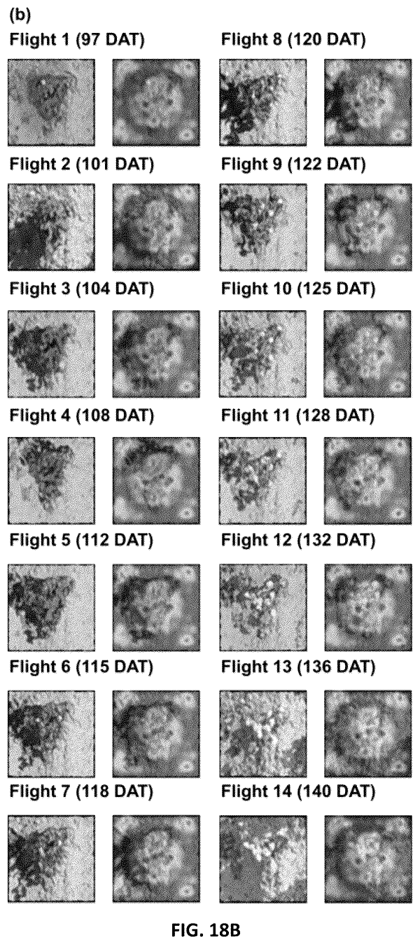

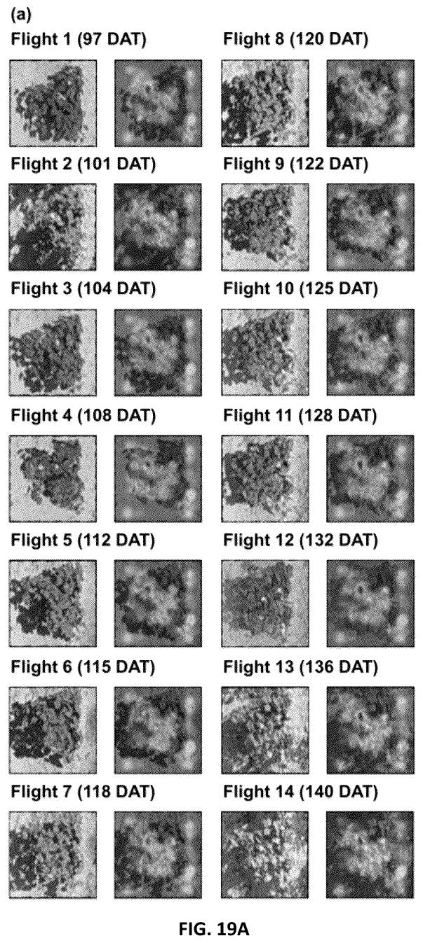

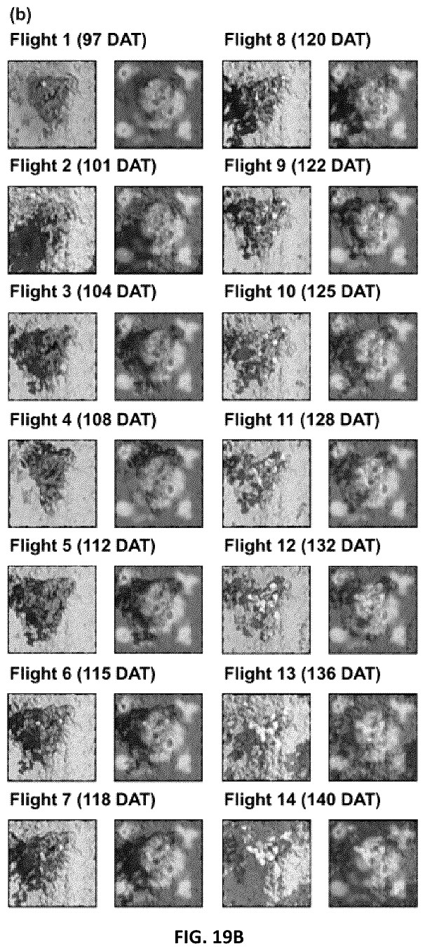

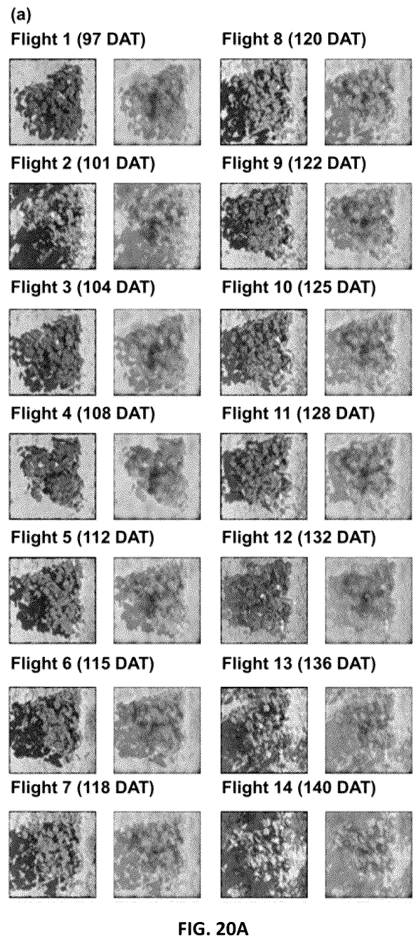

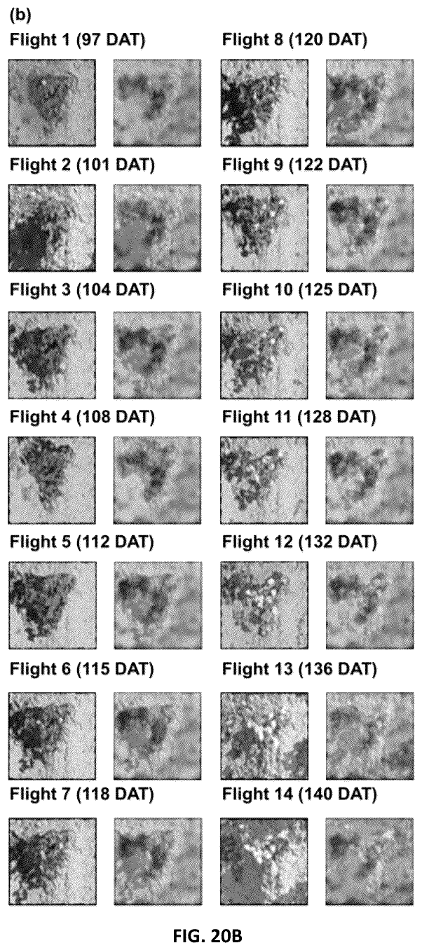

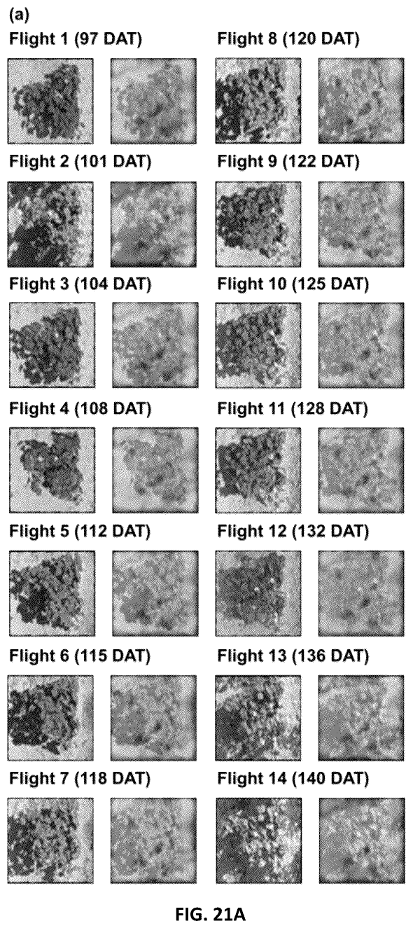

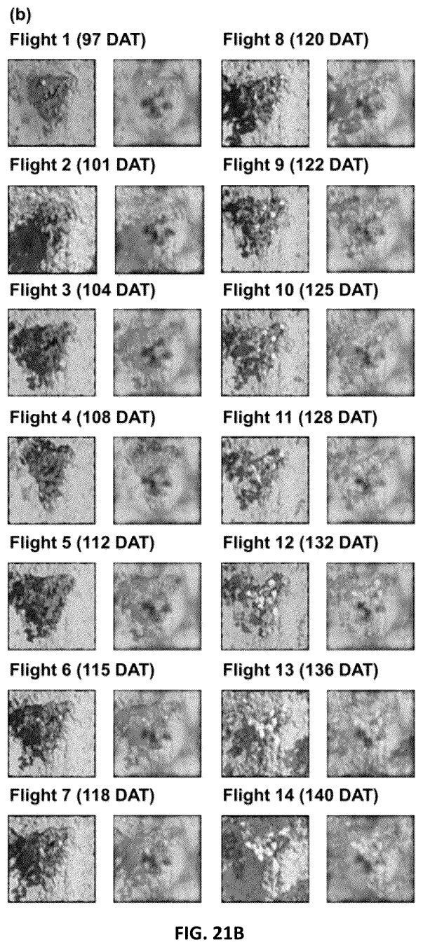

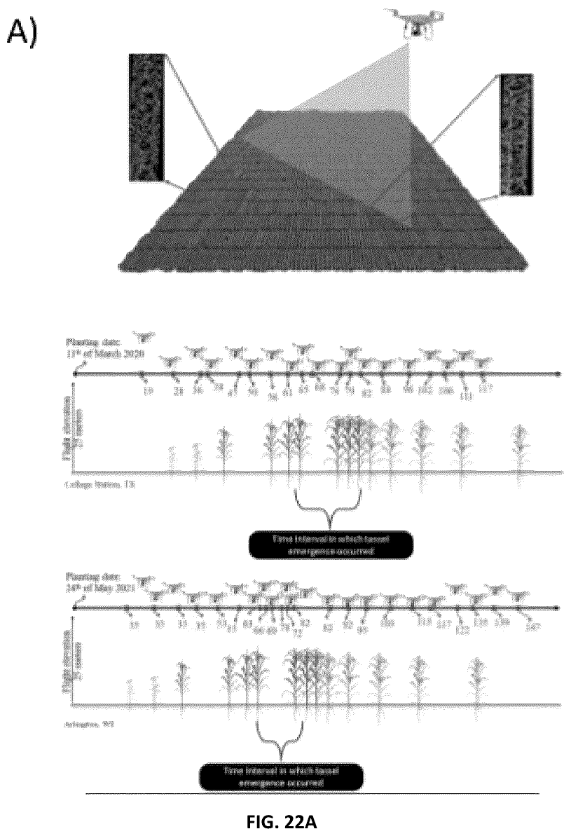

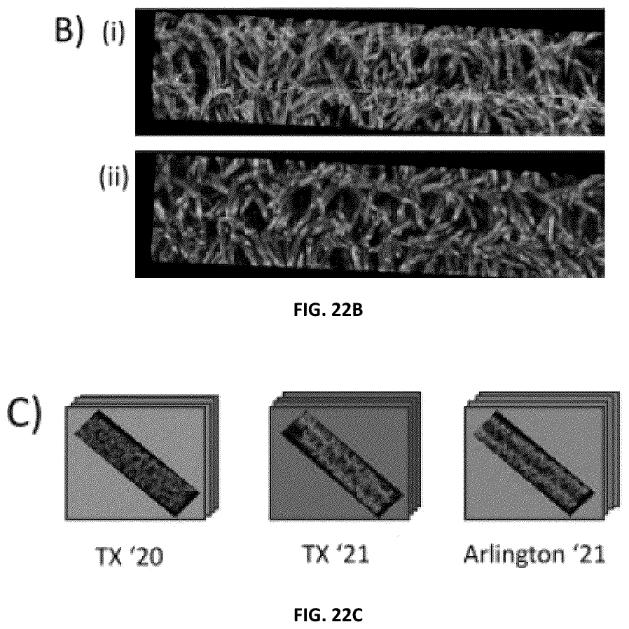

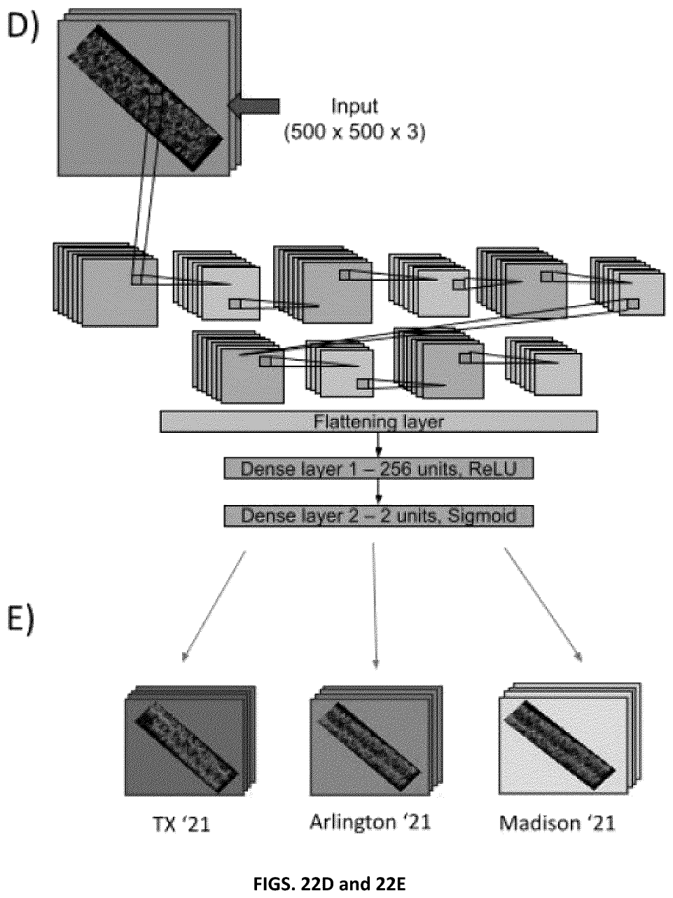

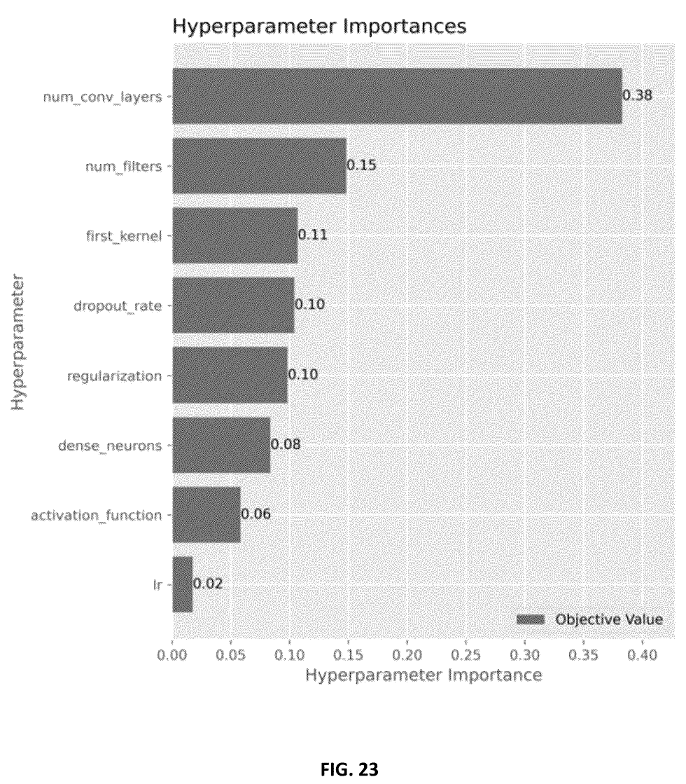

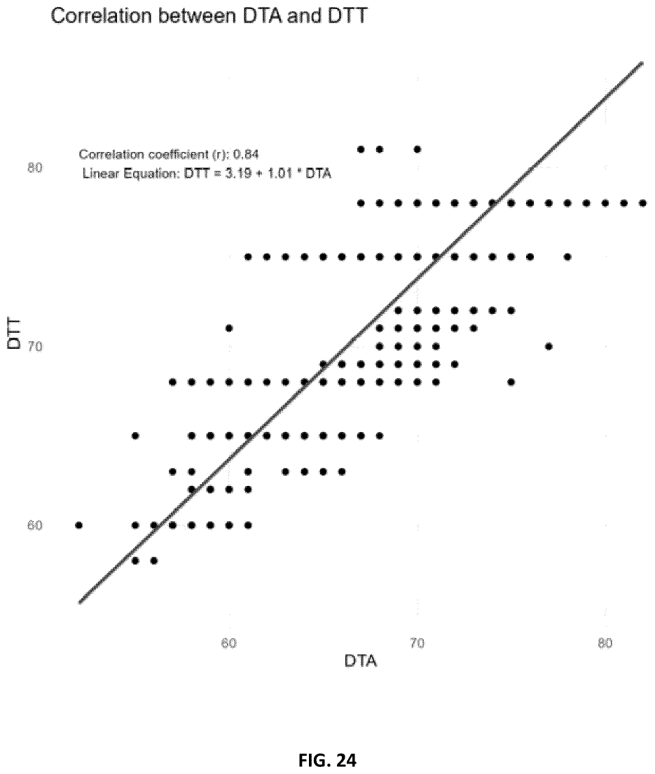

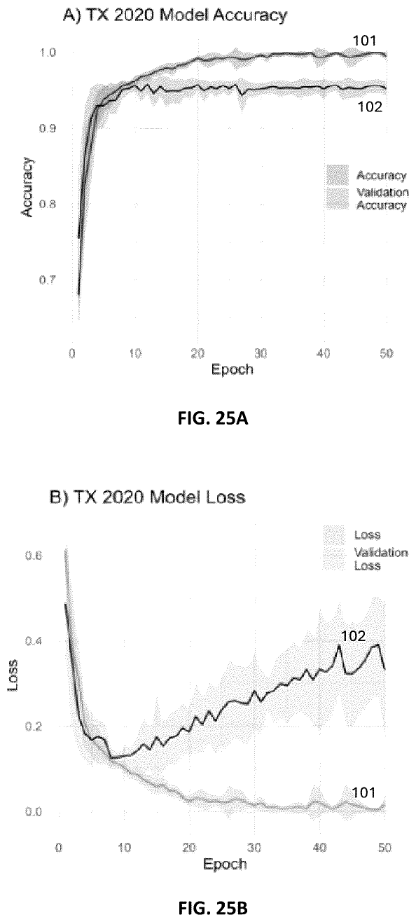

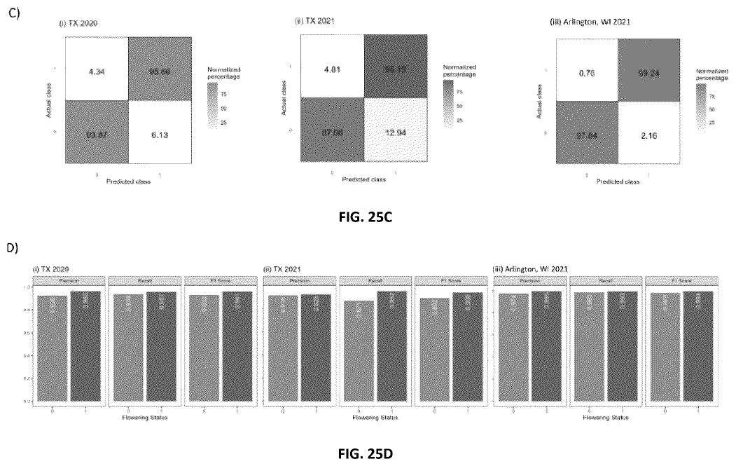

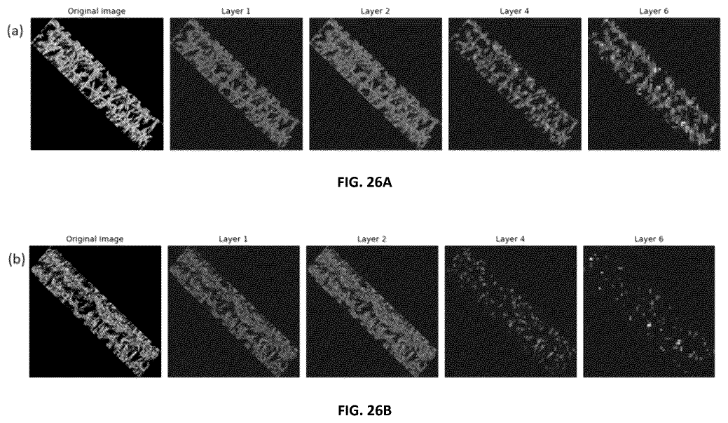

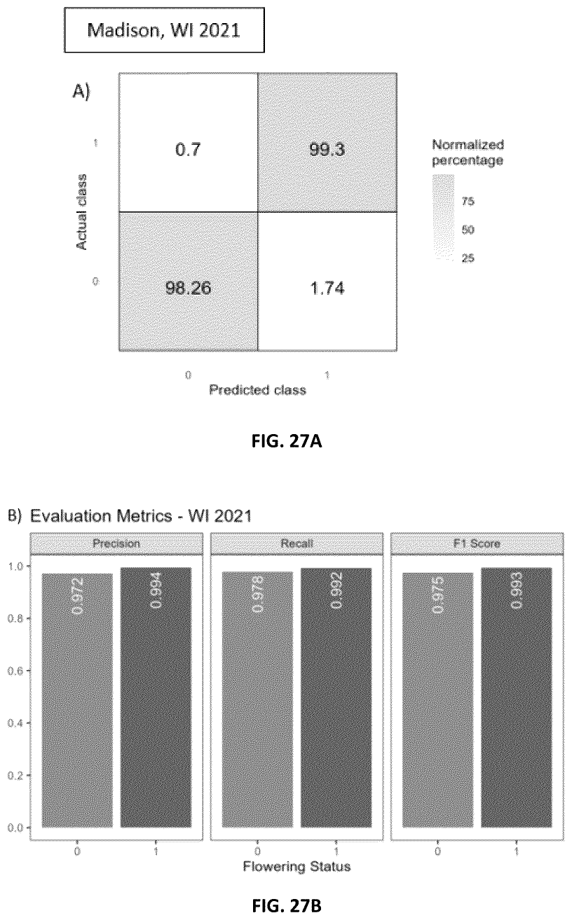

8Cshowthreetemporalphenotypesofsenescenceareshownforindividualplantsacross10ofthe14flight sselectedforsenescencescoring. Inthisfigureonly,contrast was enhanced, and soil was manually removed for illustration purposes, however this wasnotperformedforCNNtraining andtesting (rawimageswereused).FIG. 8Ashowsthatforthistemporalphenotype,senescenceprogress eduntilpermanentplantdeath.FIG. 8Bshowsthatthe stay-greenphenomenonwasobserved,wheretheplantmaintainedintermediategreennessdespite heat stress and drought. FIG. 8C shows that the temporal phenotype experienced an initialsenescentepisodebutrecoveredanddisplayedlate-seasonvigor.

FIGS. 9A-9Cshowcorrelationsofall34vegetationindices (VIs) withvisualsenescence ratings (VSRs) as shown in FIG. 9A. FIG. 9B shows the red chromatic coordinate index (RCC) displayedthehighestpositivecorrelationwithVSRs,whilethetransformednormalizeddiff erencegreenand red index (TNDGR) as shown inFIG. 9C displayed the highestmagnitude ofcorrelationwithVSRs.

FIGS. 10A-10C show plots of the first two functional principal components (FPC1and FPC2) are presented from FPCA of combined senescence-tracking metrics from experiment 1 (E1) andE2 basedonvisualsenescence ratings (VSR) asshowninFIG.10A, RCCasshowninFIG.10B, andTNDGRvegetationindexvaluesasshowninFIG.10C.Opencirclesindicatepl antsclassifiedasrapidlysenescingbasedonVSRFPC1>10.

FIGS. 11A- 11BshowpredictedFPCAscoresofvisualsenescenceratingsarepresented for all 235 single plants in experiment 1 (E1) as shown in FIG. 11A and for six selectedgenotypesandtheirrespectivereplicatesasshowninFIG.11B.

FIGS. 12A-12Cshowthegraphicaldepictionofanalysisofvariance (ANOVA) forsetting FPC1 scores of VSRs as shown in FIG. 12A, RCC as shown in FIG. 12B, and TNDGR vegetationindicesasresponsevariablesforexperiment1 (E1) asshowninFIG.12C.Opencirclesindicatere peatabilityvaluesascalculatedbyEq.3andclosedcirclesdenote

R2valuesforeachtrait.Eachpane,reportsANOVAofFPC1scorescalculateddirectlyfromthedata,wherea stherightsidereports ANOVA of CNN regression output of the top-performing model for each senescence metric.CNNtrainingandtestinghereoccurredwithinE1.

FIGS. 13A-13B show an assessment of models M1-6 according to hyperparametervariableimportancescoresdeterminedbyfunctionalanalysisofvariance (fANOVA) usin gtheOptuna Python package. Variable importances are presented in FIG. 13A. Each Optuna trial's meansquarederror (MSE) valueisindicatedbyacircleinFIG.13B. The lines atthe bottom ofFIG.13Bindicatelocalminima (improvements) astheywerediscoveredacrossthe250trials.Y- axesareidentical withineachtraitforeaseofcomparisonbetweenmodels (visual score-M1/M4,RCC- M2/M5, and TNDGR M3/M6), but within each trait, some outlier MSE values are not visible as theY-axesweretruncatedforvisualclarityofthemajoritytrialresults.

FIGS. 14A-14BshowallsixCNNs (M1-
6) wereevaluatedwith25replicationsbyusingOptuna- derivedhyperparameters. Ineachreplication,modelsweretrainedusing80% oftime-series images (TSIs) from E1 (with an internal 20% validation image set) and were tasked withoutputtingFPC1valuesfromallE2images, whichrepresentedunseenvalidationTSIs. Lossvalues (aver aged from 25 replications) are presented in FIG. 14A. Mean absolute error (MAE) values, againaveragedacross25 replications, aregiveninFIG. 14B.ForRCCandTNDGR results, some initial lossandMAEvalueswereoutliersandhencetheY-axeshavebeentruncatedforvisualclarity.

FIGS. 15A-15B show saliency/activation maps from model M4 for two plants, onewithastay-greenphenotypeasshowninFIG.15Aandtheotherwitharapidsenescencephenotype as shown in FIG. 15B. Both plants shown were from the E2 validation set that were notusedinmodeltraining.Colorsclosertoredindicateregionsofstrongactivation.

FIGS. 16A-16B show activation maps presented for M1 (visual senescence scores).

FIGS. 17A-17B show activation maps presented for M4 (visual senescence scores).

FIGS. 18A-18B show activation maps presented for M2 (RCC).

FIGS. 19A-19B show activation maps presented for M5 (RCC).

FIGS. 20A-20B show activation maps presented for M3 (TNDGR).

FIGS. 21A-21B show activation maps presented for M6 (TNDGR).

FIGS. 22A-22E show a summary of initial methods. FIG. 22A shows UAS images wereusedtogenerateorthomosaicsofthefieldacrossflightdates (asdaysafterplanting, (DAP)) forCollege Station, TXin2020andArlington,WI in2021containingimagesofmaizewithandwithout tassels. The highlighted window contains the flowering window used. FIG. 22B shows rowplots that were then cropped into images, consisting of either a (i) flowered or (ii) unflowered plot.FIG. 22CshowsimagesfromCollegeStation, Texasin2020and2021aswellasArlington, Wisconsin in 2021 were visually scored using a 0 or 1 for flowering status. FIG. 22D shows a Keras- basedCNNmodelthatwastrainediterativelyon arandomlysorted80% subsampleof162preprocessedimagesintheRGBspectra from onlyCollegeStation, Texas plots in2020alone withdimensions 500163x500pixelsx 3 (RGB).FIG. 22Eshowsmodel predictedclassesforunseen images that were withheld from training from College Station, Texas in 2021; Arlington, Wisconsinin2021;andMadison, Wisconsinin2021.

FIG. 23 shows the hyperparameter importance as determined by Optuna.

OptunaV3 was performed iteratively over 294 fifty trials examining the importance of all listedtunablehyperparameters.

FIG. 24 shows the correlation between DTT and DTA in both WI and TX in 2020 &2021.Correlationbetweendaystotasseling (DTT) anddaystoanthesis (DTA).

FIGS. 25A-

25Dshowmodeltraining&validationmethods.FIG. 25Ashowsaccuracy (line101) andvalidationaccuracy ( line102) fortheTX2020modelwhichwas trainedon80% oftheimagesfromCollegeStation, TXin2020.FIG. 25Bshowsloss (line101) andvalidationloss (line 102) for the TX 2020 model. FIG. 25C shows confusion matrices for unseen images in (i) TX2020, (ii) TX 2021and (iii) Arlington, WI2021allclassifiedusing the

TX2020model.Rows ofeachconfusionmatrixarenormalizedsuchthateachrowsumsto100%. FIG. 25Dshowsprecision, recall, andF1-scores for eachenvironment ((i) TX2020, (ii) TX2021and (iii) Arlington, WI2021) basedonfloweringstatus.

FIGS. 26A-26Bshowsaliencymaps.Activationmapsofmaizeplants, flowered/with tassels as shown in FIG. 26A and non-flowered/without tassels as shown in FIG. 26Binthecroppedimages.

FIGS. 27A-

27BshowtheMadison, WImetrics.DeeplearningmetricsforthedistincthybridsintheMadisonenviron mentthatwerenotpresentinCollegeStation, TXorArlington, WI.FIG. 27Ashowsconfusionmatrixforth eactualandpredictedclassesofmaizeimages from Madison, WI. FIG. 27B shows precision, recall, and F1-scores for maize images fromMadison, WIbasedonactualfloweringstatus.

### DETAILEDDESCRIPTION

Unlessdefinedotherwise,all technicalandscientifictermsusedhereinhavethesame meaning as commonly understood by one of ordinary skill in the art. Methods and materialssimilar or equivalent to those described herein can be used in the practice or testing of the presentdisclosure.As usedinthespecification, andintheappendedclaims, thesingularforms “a,” “an,” “the” include plural referents unless the context clearly dictates otherwise. The term “comprising” andvariationsthereofasusedhereinisusedsynonymouslywiththeterm “including” andvariationsthereo fandareopen,non- limitingterms. Theterms “optional” or “optionally” usedhereinmeanthatthesubsequentlydescribedfea ture, eventorcircumstancemayormaynotoccur, and that the description includes instances where said feature, event or circumstance occursandinstanceswhereitdoesnot.Rangesmaybeexpressedhereinasfrom “about” oneparticularvalu e, and/orto “about” anotherparticularvalue.Whensucharangeisexpressed, anaspectincludesfromtheo neparticularvalueand/ortotheotherparticularvalue.Similarly, whenvaluesareexpressedasapproximat ions,by useoftheantecedent “about, “itwillbeunderstoodthattheparticularvalueformsanotheraspect.Itwill befurtherunderstoodthattheendpointsofeachoftherangesaresignificantbothinrelationtotheotheren dpoint, andindependentlyoftheotherendpoint.

Theterm” artificialintelligence “isdefinedhereintoincludeanytechniquethatenables oneormorecomputingdevicesorcompingsystems (i.e.,amachine) tomimichumanintelligence.Artificia lintelligence (Al) includes,butisnotlimitedto, knowledgebases, machinelearning, representationlearni ng, anddeeplearning. Theterm” machinelearning “isdefinedhereintobeasubsetof

Althatenablesamachinetoacquireknowledgebyextracting patternsfromrawdata.Machinelearningtechniquesinclude,butarenotlimitedto,logisticregression,su pportvectormachines (SVMs),decisiontrees, NaïveBayesclassifiers, andartificialneuralnetworks. The term “representation learning” is defined herein to be a subset of machine learningthatenablesamachinetoautomaticallydiscoverrepresentationsneededforfeaturedetection, prediction, or classification from raw data. Representation learning techniques include, but are notlimitedto, autoencoders. Theterm “deeplearning” isdefinedhereintobeasubsetofmachinelearning that that enables a machine to automatically discover representations needed for featuredetection,prediction,classification,etc.usinglayersofprocessing.Deeplearningtechniquesincl ude,butarenotlimitedto, artificialneuralnetworkormultilayerperceptron (MLP).

Machine learning models include supervised, semi-supervised, and unsupervisedlearning models. Inasupervisedlearningmodel, themodel learnsafunctionthatmapsaninput (alsoknownas featureorfeatures) toanoutput (alsoknownas targetortargets) during trainingwith a labeled data set (or dataset). In an unsupervised learning model, the model learns patterns (e.g., structure,distribution,etc.) withinanunlabeleddataset. Inasemi-supervisedmodel, the model learns a function that maps an input (also known as feature or features) to an output (alsoknownastargetortarget) duringtrainingwithbothlabeledandunlabeleddata.

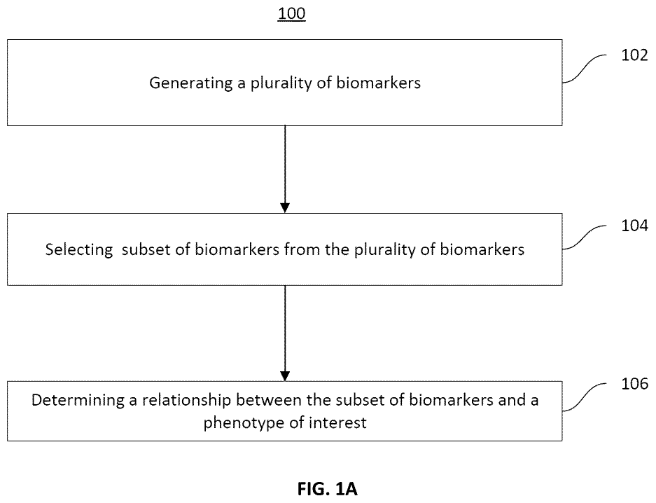

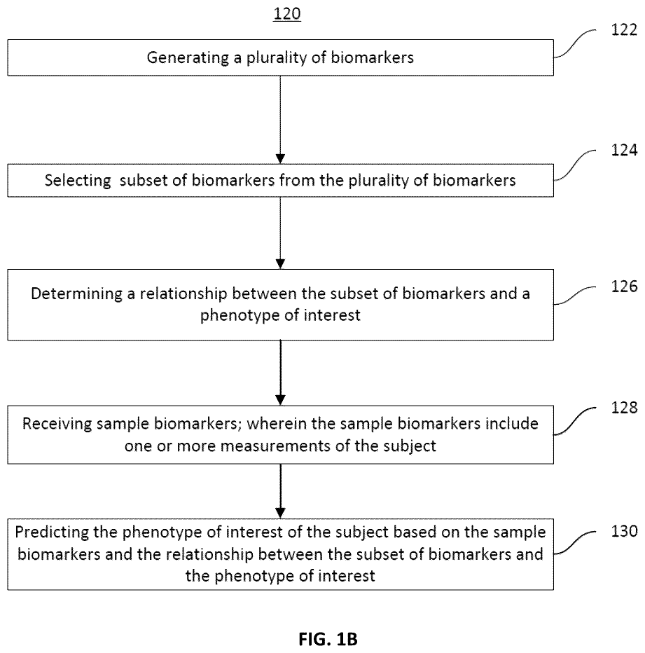

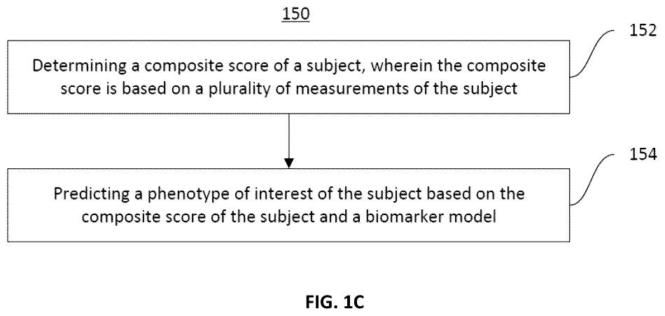

With reference to FIG. 1A, an example method 100 of selecting biomarkers isshownaccordingtoimplementationsofthepresentdisclosure.

Atstep102, themethodincludesgeneratingapluralityofbiomarkers. Thebiomarkers canbeanybiologicalmeasurementofasubjectincludingthoseimagedorremotelysensed.Optionally,th epluralityofbiomarkerscanincludeoneormoreof: geneticdata,metabolomic data or proteomic data. Alternatively or additionally, the plurality of biomarkers canincludetemporally (longitudinally) measuredbiomarkers.

Insomeimplementations, thepluralityofbiomarkersincludesremotelysenseddata.A nexampleofremotelysenseddataisimagedataandfeaturesextractedfromthisimagedataatonepointin timeortemporally.As usedherein, “imagedata” canrefertoanykindofimaging, including imaging performed by unmanned aerial vehicles (UAVs) and satellites, as well asimagingperformedusinganywavelengthofelectromagneticradiation (e.g., infraredimaging). Additionaldescriptionofimagingtechniquesthatcanbeusedtoacquirebiomarkersinimplementations of the present disclosure are described in Example 1, as well as Appendices A-Ehereto.

Optionally, imagedatacanbedecomposedintoapluralityofimagefeatures, where the image features can be estimated from the image data. The image features can representdifferent subjects in an image (e.g., each of the organisms that is in the image) or different parts ofsubjects inanimage (e.g., eachpartofeachorganismintheimage).Alternatively oradditionally, theimagefeaturescanrepresentthespectralreflectance,position, orientationand/orsize oftheorganismsintheimage.Asyetanotherexample, theimagefeaturescanrepresentthespectralreflect ance, position, orientation, and/or size of each part of each organism in the image. Any or alloftheimagefeaturescanbeestimatedfromtheimagedata.

Optionally, machine learning can be used to generate the biomarkers. In someimplementations, thepluralityofbiomarkerscanbegeneratedusingaconvolutionalneuralnetw ork.

Atstep104, the method includes selectinga subsetofbiomarkers fromtheplurality of biomarkers. Optionally, it should be understood that in some implementations themethod100canbeperformedwithoutperformingstep104.

Insomeimplementationsofthepresentdisclosure,selectingthesubsetofbiomarkersc anincludeselectingbiomarkersbasedacorrelationbetweenabiomarkeroftheplurality of biomarkers with a different biomarker of the plurality of biomarkers. For example, if thecorrelation between two biomarkers is too great (e.g., 1.0), then one of the two biomarkers can beconsideredtoberedundant, andthetwobiomarkerscanbeconsiderednon-uniquebiomarkers. Biomarkersthatare not completelyredundantwithother biomarkers canbeconsidered “unique” biomarkers. In some implementations of the present disclosure, step 104 can include selecting thesubsetofbiomarkerstosothatthesubsetofbiomarkersincludesonlyuniquebiomarkers.

Optionally selecting the subset of biomarkers can include selecting heritable orrepeatablycollectablebiomarkersfromthepluralityofbiomarkers.

At step 106, the method further includes determining a relationship between thesubsetofbiomarkersandaphenotypeofinterest.

In some implementations of the present disclosure, determining the relationshipbetween the subset of biomarkers and the phenotype of interest can be based on an Al or machinelearningtestofpredictionability. Non- limitingexamplesofmachinelearningtestsofpredictionability thatcanbeusedinimplementations ofthepresentdisclosureincludealassotest, aridgeregressiontest, aBayes Btest, and/ororarandomforestregressiontest.

Optionally, theAlormachinelearningtestcanfurtherincludecrossvalidation.

As non-limiting examples, the phenotype of interest can be a plant phenotype. Example plant phenotypes include plant disease resistance, plant composition, plant performance,plant height and/or plant yield. Alternatively, or additionally, the phenotype of interest can includeadiseaseorriskofdisease.

In some implementations, the method 100 can further include determining anoptimal management strategy for improving health, production or other value-added trait for asubject. Therelationshipbetweenthesubsetofbiomarkersandthephenotypeofinterestdetermine datstep106canbeusedtopredicthowchanges tothebiomarkerscanaffectthephenotypeofinterest.

Optionally, theoptimalmanagementstrategycanbeconfiguredbasedonbiomarkerst ochangethephenotypeofinterest. Asanon-limitingexample, increasingordecreasing the nutrients available to the subject (e.g., by watering or fertilizing a plant subject) canaffect biomarkers that are related to nutrition, and thereby improve the health of the subject (e.g., theyieldofaplantsubject).

In some implementations, the method 100 can further include predicting achanceofsuccessofthephenotypeofinterestinaselectivebreedingprogram.

With reference to FIG. 1B, implementations of the present disclosure can includemethodsofpredictingphenotypesofinterestbasedonsamplebiomarkersobtainedfromasubjec t.

Atstep122, themethodincludesgeneratingapluralityofbiomarkers. Theplurality of biomarkers can be generated using any of the methods described with reference tostep102inFIG.1A.

At step 124, the method includes optionally selecting a subset of biomarkers fromthepluralityofbiomarkers. Thesubsetofbiomarkerscanbeselectedusinganyofthemethodsdescrib edwithreferencetostep104inFIG.1A.

At step 126, the method can further include determining a relationship betweenthesubsetofbiomarkersandthephenotypeofinterest.Again, therelationshipbetweenthesub set of biomarkers and the phenotype of interestcan be determined using any of the methodsdescribedwithreferencetostep106inFIG.1A.

Atstep128, themethodcanfurtherincludereceivingsamplebiomarkers. Thesample biomarkers can include one or more measurements of the subject. Again, the subject canbeanyorganism, includinganyplantoranimal.

Atstep130, themethodcanfurtherincludepredictingthephenotype(s) ofinterest of the subject based on the sample biomarkers and the relationship between the subset ofbiomarkersandthephenotype(s) ofinterest.Itshouldbeunderstoodthatthemethodsandsystems described herein can be applied to large number of biomarkers, for example at least thirtybiomarkers.Largenumbersofbiomarkerscanbeusedto “predicttheorganism” bypredictingmorep henotypes, and/orpredictingthosephenotypesmoreaccurately, thanconventional predictions (that may use a single biomarker to predict a single phenotype, for example correlatingthebiomarkerofplantheightwiththephenotypeofplantyield).

In some implementations, the method can further include predicting a chance ofsuccessof the subject in aselective breedingprogram. Asused herein, the chanceofsuccessof asubject(s) inaselectivebreedingprogramcanbethechancethatoffspringofbreedingwiththesubject(s) exhibitthedesiredphenotype(s) ofinterestofthesubject.

WithreferencetoFIG.1C,implementationsofthepresentdisclosureincludemethods of predicting one or more phenotypes of interest of a subject using a composite score. Asused herein, a composite score can represent one or more measurements of a subject.

Optionally,ameasurementcanbea measurementofbiomarkers,wherethebiomarkerscanbedeterminedusinganyofthemethods100,120 describedwithreferencetoFIG.1Aand1B.Thiscompositescorecouldalsocontainothermeasurementss uchastemporaldata,geneticdata, metabolomicdata,proteomicdataoroutputsofmodelsthatusesuch measurements.

At step 152, the method can include determining a composite score of a subject, where the composite score is based on a plurality of measurements of the subject. Optionally, themeasurementscanincludeanyofthebiomarkersdescribedhereinoranyothermeasuresdescribedh erein.

Atstep154, themethodcanincludepredictingoneormorephenotype(s) ofinterestoft hesubjectbasedonthecompositescoreofthesubjectandabiomarkermodel. Thebiomarker model can be generated based on the subset of biomarkers described with reference toFIGS. 1A- 1B.Optionally, thebiomarkermodelcanincludebiomarkersselectedbasedonacorrelationbetweenabio markerofapluralityof biomarkerswithadifferentbiomarkerofthepluralityofbiomarkers.

WithreferencetoFIG.2,implementationsofthepresentdisclosureincludesystems for implementing any of the methods 100, 120, 150 shown and described with referencetoFIGS. 1A-1C.

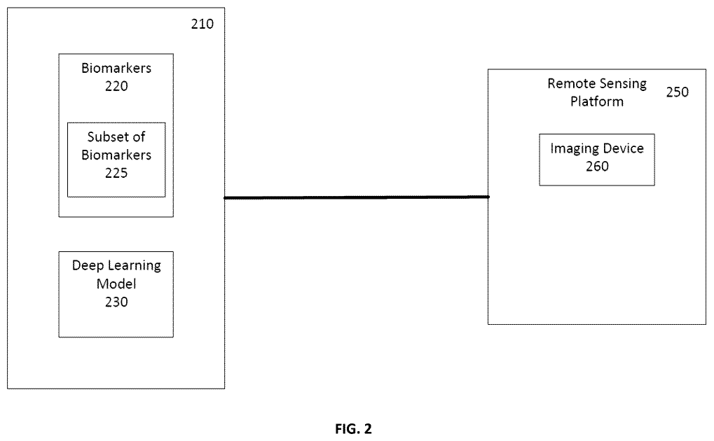

FIG. 2illustratesasystem200forcapturingbiomarkersanddeterminingrelationshipsbetweenthosebio markersandaphenotypeofinterest.

Thesystem200showninFIG.2canincludearemotesensingplatform250.

The remote sensing platform 250 can include an imaging device or other devicetocaptureanorganism orpartofanorganism. Theimaging device260canbeconfiguredto acquire images or point measures using any wavelength(s) of electromagnetic radiation. As non- limitingexamples, theimagingdevice260canbe adigitalcameraconfiguredtoacquirevisiblelight, and/oranear- infraredcameraconfiguredtocapturenear- infraredimagesoraspectrophotometertocapturemeasurementofasinglepoint.

Optionally, theremotesensingplatform250canincludeanunmannedaerialvehicle (e.g., a drone with a rotary or fixed wing design). Alternatively, the remote sensing platform250canincludeamannedaerialvehicle (e.g., ahelicopterorplane).Alternatively, theremotesens ing platform250canincludeasatellite. Asyetanotherexample, theremotesensingplatform250 can include a ground vehicle or ground robot in a field, greenhouse or growth chamber. As yetanotherexample, theremotesensingplatform250canincludeamedicalimagingdeviceorhandheld camera. Further, implementations of the present disclosure can include any combinationof remote sensing platforms 250. The remote sensing platform 250 can be configured to image anyorganismorgroupoforganisms.For example, theremotesensing platform250canbeconfiguredtomeasureoneormorefieldsofcrops.Alternativelyoradditionally, there motesensingplatform250canbeconfiguredtoimagetheorganismsatdifferentpointsintime,referredto hereinas “temporally-spacedimages” (e.g., everyhour,day,weekormonth).

Thesystem200canfurtherincludeacomputingdevice210. Thecomputingdevice210 canincludeanyorallofthecomponentsshownanddescribedwithreferencetothecomputingdevice300 showninFIG.3. Thecomputingdevice210canbe operablyconnectedtothe remotesensing platform 250 (e.g., by a wireless or wirednetwork). In someimplementationsof the present disclosure, the system 200 can include more than one remote sensing platform 250 (e.g., agroupofdrones) and/ormorethanonecomputing device210 (e.g., aserver),wherethecomputingdevicesandremotesensingplatforms250arenetworked togethertoimplementthemethods100,120,150describedwithreferencetoFIGS. 1A-1C.

Thecomputingdevice210canoptionallybeconfiguredtoperformanyorallofthemeth odsshownanddescribedwithreferencetoFIGS. 1A-1C. Thecomputingdevice210canbeconfigured to storea plurality of biomarkers 220and to select asubset of biomarkers225 fromthe plurality of biomarkers. For example, the image data collected by the imaging device 260 of caninclude the plurality of biomarkers, and the computing device 210 can be configured to identify theplurality of biomarkers from theimage data and determinea subset of thosebiomarkers. In some implementationsofthepresentdisclosure, thecomputingdevice210canfurtherstoreanAlmodel,machi nelearningmodeland/ordeeplearningmodel.Asshownin FIG. 2, theexamplesystem200includesadeeplearningmodel230, whichcanbeusedtogenerateapluralityofbiomarkers and/or select the subset of biomarkers from the plurality of biomarkers, as describedwithreference toFIGS. 1A-1C. Itshouldbe understoodthatthe othermachine learning modelsdescribedhereincanbe usedinconjunctionwith, orinplaceof, thedeeplearning model 230.

Alternatively or additionally, the computing device 210 can determine arelationshipbetweenthesubsetofbiomarkersandthephenotypeofinterest.

In some implementations of the present disclosure, computing device 210 can beconfiguredtocontroltheremotesensingplatform250.For example, theremotesensingplatform250 can be controlled remote based on the relationship between the subset of biomarkers and thephenotype of interest. Controlling the remote sensing platform 250 can optionally include changingthefrequencythatimagingis performedusing theimaging device260, and/orcausing theremotesensingplatform250toacquireimagesofspecificorganismsorgroupsoforganisms.

Asyetanotherexample, theremotesensingplatform250canbeconfiguredtoacquire biomarkers of a group of organisms, which the computing device 210 can use to identify asubset of biomarkers that can be used for prediction (e.g., according to the method described withreferencetoFIG.1Band1C). Theremotesensingplatform250canbeconfiguredtoimageasecondorg anismorsubsetoforganisms, andpredictaphenotypeofthat organismorsubsetoforganisms using the relationship between the phenotype of interest and the subset of biomarkers.

As another example, implementations of the present disclosure can be applied topredicting agricultural yield for a given crop (e.g., maize). A UAV can be configured to fly over a firstmaize field once or throughout growth to acquire many biomarkers. The computing device 210 canusesome or asubsetofthese biomarkers to buildanalgorithm thatcanpredictthegrainyieldphenotypeofthefirstmaizefield (anexamplephenotypeofinterest, disease wouldbeanotherexample). TheUAVcan be flownovera second maize fieldand acquireimagingdata including thebiomarkers detectedfromthefirstfield.Basedonthevaluationofbiomarkersfromthefirstfield, the computing device 210 can estimate the grain yield of the second maize field at any point in thegrowingseason.Implementationsofthepresentdisclosureallowforearlierandbetterpredictionofth e phenotype(s) ofinterest.Implementations of the presentdisclosure alsoallowtheselection ofbiomarkersthatpredictthephenotype(s) ofinterest, thuspredictionsbasedonthesubsetofbiomarkers canbemoreaccuratethanpredictionsbasedonconventionalassociatedphenotypesorroutinecollected biomarkers (e.g., plantheightorsizealone).Thus, implementationsofthepresent disclosure can allow for improved predictions of biological or agricultural productivity usingpreviouslyunknownbiomarkers.Further, implementationsofthepresentdisclosurecanallowforus es of biomarkers and combinations of biomarkers that were previously not known to correlate tocertain phenotypes (e.g., new ways to measure a plant that will cause or correlate to its yield). Stillfurther, implementationsofthepresentdisclosureallowforgeneratingnewbiomarkersthatcanbesp ecifictocertainorganisms (e.g., atypeofwheat).And,stillfurther,implementationsofthepresentdisclosu reenabletheselectionanduseofalluniquebiomarkers (“saturatingthephenotype” as describedinExample1herein).Alternatively, thesebiomarkerscanbeusedininplants, animals, microbest opredictimprovedmanagementstrategiestowardsprecisionagriculture.Alternatively, thesebiomarker scouldbeusedinplants, animals, microbesorhumanstopredictdiseasebetter orearlier.Alternatively, thesebiomarkerscouldbeusedinplants, animals, microbesorhumanstotrackoru nderstandbiologicalpathwaysandprocesses.

Alternatively these biomarkers could be used in animals or humans to predict improved healthinterventions (e.g. “personalizedmedicine).

Thus, implementationsofthepresentdisclosureenableimprovementstosensingandmonitoringofagric ulturalyields.

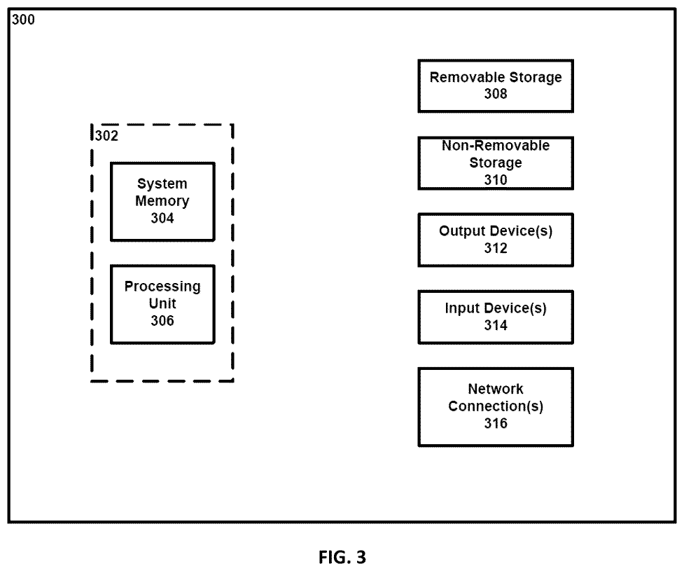

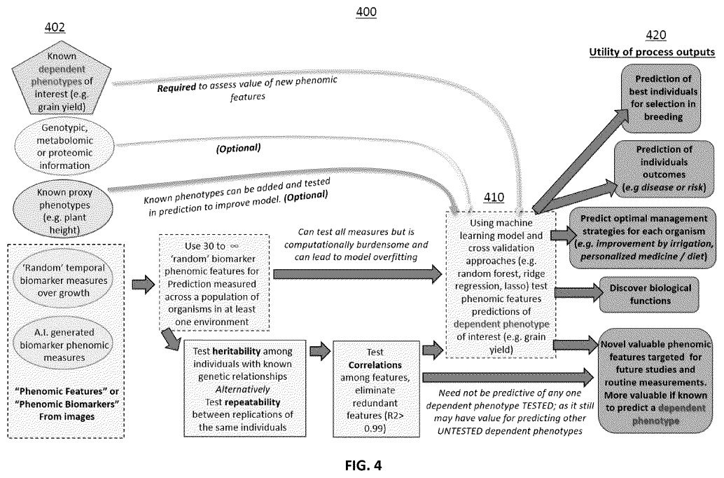

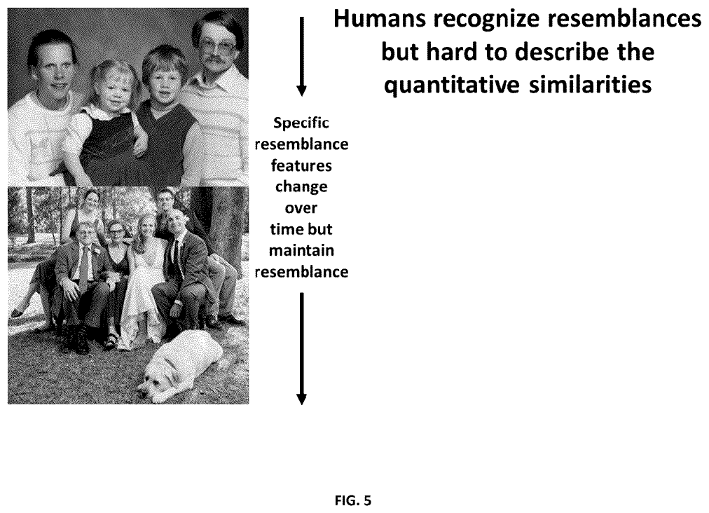

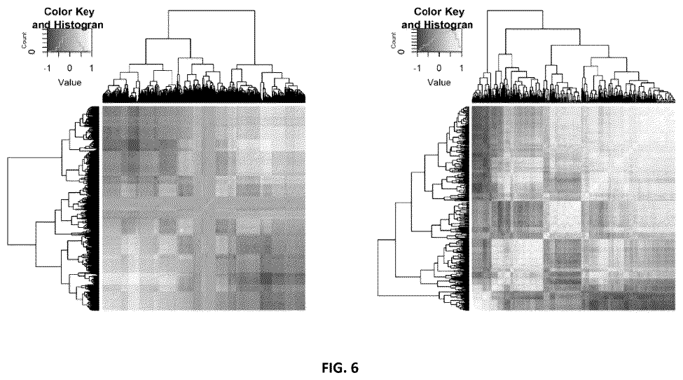

It should be appreciated that the logical operations described herein with respecttothevariousfiguresmaybeimplemented (1) asasequenceofcomputerimplementedactsorprog rammodules (i.e.,software) runningonacomputingdevice (e.g., thecomputingdevicedescribed in FIG. 3), (2) as interconnected machine logic circuits or circuit modules (i.e., hardware) within the computing device and/or (3) a combination of software and hardware of the computingdevice. Thus, the logical operations discussed herein are not limited to any specific combination ofhardware andsoftware. Theimplementationis a matterofchoicedependent on the performanceandotherrequirementsofthecomputingdevice.Accordingly, thelogicaloperationsdescrib edhereinarereferredtovariouslyasoperations,structuraldevices, acts, ormodules. Theseoperations, structural devices,actsandmodules maybeimplementedin software, infirmware, inspecial purpose digital logic, and any combination thereof. It should also be appreciated that more or fewer operations may be performed than shown in the figures and described herein. Theseoperationsmayalsobeperformedinadifferentorderthanthosedescribedherein.

ReferringtoFIG.3,anexamplecomputingdevice300uponwhichthemethodsdescribe dhereinmaybeimplementedisillustrated.Itshouldbeunderstoodthattheexamplecomputingdevice30 Oisonlyoneexampleofasuitablecomputingenvironmentuponwhichthemethodsdescribedhereinmay beimplemented.Optionally, thecomputingdevice300canbeawell-known computing system including, but not limited to, personal computers, servers, handheldorlaptopdevices, multiprocessorsystems, microprocessor- basedsystems, networkpersonalcomputers (PCs),minicomputers, mainframecomputers,embeddedsy stems, and/ordistributedcomputingenvironmentsincludingapluralityofanyoftheabovesystemsordevi ces.Distributedcomputingenvironmentsenableremotecomputingdevices, whichareconnectedtoaco mmunicationnetworkorotherdatatransmissionmedium, toperformvarioustasks. Inthedistributedco mputingenvironment, theprogrammodules, applications, andotherdatamaybestoredonlocaland/orre motecomputerstoragemedia.

Initsmostbasicconfiguration,computingdevice300typicallyincludesatleastoneproc essingunit306andsystemmemory304.Dependingontheexactconfigurationandtypeof computing device, system memory 304 may be volatile (such as random access memory (RAM)), non- volatile (suchasread- onlymemory (ROM), flashmemory,etc.), orsomecombinationofthetwo.Thismostbasicconfigurationisil lustratedinFIG.3bydashedline302. Theprocessingunit306maybeastandardprogrammableprocessort hatperformsarithmeticandlogicoperationsnecessary for operation of the computing device 300. The computing device 300 may also include abusorothercommunicationmechanismforcommunicatinginformationamongvariouscomponentsof thecomputingdevice300.

Computingdevice300mayhaveadditionalfeatures/functionality.For example,comp utingdevice300mayincludeadditionalstoragesuchasremovablestorage308andnon-removable storage 310 including, but notlimited to, magnetic or optical disks or tapes. Computingdevice 300 may also contain network connection(s) 316 that allow the device to communicate withother devices. Computing device 300 may also have input device(s) 314 such as a keyboard, mouse,touchscreen,etc.Outputdevice(s) 312suchas adisplay,speakers,printer,etc.mayalsobeincluded. Theadditional devices may beconnectedto the bus in order to facilitate communication of data among the components of the computing device 300. All these devices are well known intheartandneednotbediscussedatlengthhere.

Theprocessingunit306maybeconfiguredtoexecuteprogramcodeencodedintangible, computer-readable media. Tangible, computer-readable media refers to any media that iscapableofproviding datathatcauses thecomputingdevice300 (i.e.,amachine) tooperateinaparticular fashion. Various computer-readablemediamay be utilized to provideinstructions to theprocessing unit306 for execution. Example tangible,computer- readablemediamay include, butisnotlimitedto, volatilemedia, non- volatilemedia, removablemediaandnon- removablemediaimplementedinanymethodortechnologyforstorageofinformationsuchascomputerr eadableinstructions, datastructures,programmodulesorotherdata.Systemmemory304,removablesto rage 308, and non-removable storage 310 are all examples of tangible, computer storage media.Examplesoftangible,computer- readablerecordingmediainclude,butarenotlimitedto, anintegratedcircuit (e.g., field- programmablegatearrayorapplication-specificIC),aharddisk,anoptical disk,amagneto-optical disk,a floppy disk, amagnetic tape, a holographic storage medium, asolid- statedevice, RAM,ROM,electricallyerasableprogramread- onlymemory (EEPROM),flashmemoryorothermemorytechnology,CD- ROM, digitalversatiledisks (DVD) orotheropticalstorage,magneticcassettes,magnetictape,magneticdis kstorageorothermagneticstoragedevices.

Inanexampleimplementation, theprocessingunit306mayexecuteprogramcodestor edinthesystemmemory 304. For example, thebusmaycarry datatothesystemmemory304,fromwhichtheprocessingunit306receivesandexecutesinstructions.Th edatareceived by the system memory 304 may optionally be stored on the removable storage 308 or thenon-removablestorage310beforeorafterexecutionbytheprocessingunit306.

Itshouldbeunderstoodthatthevarioustechniquesdescribedhereinmaybeimpleme nted in connection with hardware or software or, where appropriate, with a combinationthereof.

Thus, the methodsandapparatusesofthepresentlydisclosedsubjectmatter, or certainaspectsorportionsthereof,maytaketheformofprogramcode (i.e.,instructions) embodiedintan giblemedia,suchasfloppydiskettes,CD-ROMs,harddrives, oranyothermachine-readablestorage medium wherein, when the program code is loaded into and executed by a machine, suchasacomputingdevice, themachinebecomesanapparatusforpracticingthepresentlydisclosed subjectmatter. Inthecaseofprogramcodeexecutiononprogrammablecomputers, thecomputingdevice generallyincludesaprocessor, astoragemediumreadablebytheprocessor (includingvolatileandnon- volatilememoryand/orstorageelements), atleastoneinputdevice, andatleastoneoutputdevice.Oneor moreprogramsmayimplementorutilizetheprocessesdescribedinconnectionwiththepresentlydisclose dsubjectmatter,e.g., throughtheuseofanapplicationprogramminginterface (API), reusablecontrols, ort helike.Suchprogramsmaybeimplemented in a high level procedural or object-oriented programming language to communicatewithacomputersystem.However, theprogram(s) canbeimplementedinassemblyormach inelanguage,ifdesired. Inanycase, thelanguagemaybeacompiledorinterpretedlanguageanditmaybeco mbinedwithhardwareimplementations.

### EXAMPLES

The following examples are put forth so as to provide those of ordinary skill in theartwithacompletedisclosureanddescriptionofhowthecompounds, compositions, articles, devicesa nd/ormethodsclaimedhereinaremadeandevaluated, andareintendedtobepurelyexemplary and are not intended to limit the disclosure. Efforts have been made to ensure accuracywith respect to numbers (e.g., amounts, temperature, etc.), but some errors and deviations shouldbeaccountedfor. Unless indicatedotherwise, partsareparts byweight, temperatureisin® Corisatambienttemperature, andpressureisatornearatmospheric.

### Example 1:

Anexampleimplementationofthepresentdisclosureincludessystemsandmethodsfo rselectingbiomarkersandpredicting phenotypes. Theexampleimplementationofthepresentdisclosuredescribedhereincanovercomechall engesofconnectinganorganism′sgenotype or genome, to their phenotype and their phenome [1- 6]. Overcoming the challenges ofconnectinganorganismsgenotypeorgenometothatorganismsphenotypeandphenomehassignifican timplicationsforaddressingenvironmentalandpolicychallenges.For example,sustainablefoodsecurity underclimatechange.

WithreferencetoFIG.4,acomputer- implementedmethod400ofperformingpredictionsisshownaccordingtoanexampleimplementationo fthepresentdisclosure. Themethod400canreceiveinputs402includingknowndependentphenotypes, genotypicinformation, known proxy phenotypes, random temporal measures (temporal biomarkers), and Al generatedphenomicmeasures (Alextractedbiomarkers). Themethod400canfurtherincludeusing a machine learning model 410 to test phenomic features (biomarkers) and predictions of thedependent phenotype of interest. The outputs 420 of the machine learning model 410 can includeanyorallofthefollowing: predictionofbestindividualsforselectioninbreeding, predictionofindivi dual outcomes, predictionofoptimalmanagementstrategiesforeachorganismorgroupoforganisms, new understanding of biological processes and relationships, and identifying novel andvaluablephenomicfeaturestargetedforfuturestudiesandroutinemeasurements.

Asanexample, thehumangenomehasbeensequenced [7,8], andanotherexample is the Arabidopsis 2010 project [9], to determine the function of all genes in Arabidopsis.

Inphenomics, ananalogouschallengeistosaturateaphenomeofanindividualorpopula tion, findingallpossiblebiomarkers.Asusedinthepresentexample, “saturatingthephenome” canmeant hatnonewbiomarkermeasurementsofanorganismcanbemadethatcannotalreadybepredictedfromot hermeasures. Inotherwords, thedepthofphysiologicalresponsesoftheorganismacrossanypossibleenvi ronment, management, orscenario (withinreason) canbereducedtonumbersandpredictedatany pointintime.

Thiscanrequirenotonlypredictingnature (thegenome), butnurture (theresponseofanorganismtoenvir onmentandmanagement conditions) as well as interactions between the two [10-16]. Controlled environments (suchasgreenhouses,growthchambers,animalcontrolfacilities, orurbanenvironments) r educeenvironmental,management, anderrornoiseinphenomicmeasurementsandaddalevelofprecisi on to understanding the phenome. Controlled environments also have the potential capacitytocollectmorebiomarkermeasurementswithinenvironmentsthroughfullyautomatedsystems thatareimpracticalunderfieldconditions [17, 18].However,highlycontrolledsystemscannotscreenthenumberordiversityofenvironmentsorthenu mberofindividualsneededtosaturatephenomes,soanimplementationsofthepresent disclosurecanbenefitfieldsystemswhichcanincludeagreaterdiversityofenvironmentsand/orgreatern umberofindividuals.

Phenomicselectionapproachescanoutperformgenomicselection, thegoldstandar dinpredictingphenotype, andwithlesscost. [33].For example,usinglaboratorynearinfraredreflectanc espectroscopy (NIRS) measuresofwheatgrainandpoplarbark. Incredibly,throughcross- validation, theyshowedthatusingmanybiomarkerreflectancebandsfromtheseproducts could accurately predict yields in a population [33-36]. In the case of NIRS, thousands of reflectancebandsaretreatedsimilarlytogenomicmarkersingenomicselection;quasi- independentrepeatablemeasuresseparatingindividuals. Infieldstudieshowever, evenwiththemost intense measurement of currently known phenotypes (e.g., height, leaf angle, flowering time,etc.) arichenoughdatasetcannotbecreatedwithcurrentmethods torepeatablyseparateandpredicteachgenotype.Furthermore, mosttraditionalphenotypesareendpoi ntmeasures, whereinteractionswithenvironmentareintegratedovertheorganismslife.Unoccupiedaeri alsystems (e.g., theUAVsdescribedabove) providenewwaystocollectmassiveamountsofbiomarkerdata across large numbers of individuals and environments, extracting novel features (biomarkers)

[22]. Yetunlesshyperspectralbandsareused, vegetationindexesarecalculated, oralgorithmsextractprev iouslyunknownbiomarkers, challengesremainincollectingenoughfeaturestouseinphenomic prediction. Using temporal collection of biomarkers, can be multiplicative in the numberofphenomicfeaturesandallowearliernearreal-

timepredictionability. [37] Thisshowedthattemporalphenomicpredictioncanindeedperformsimilarly, perhapsbetterthangenomicprediction.However, evenwiththelargestreportedUASdataset,only896ph enomicfeatureswereextractedcomparedwith11,000genomicmarkers.Phenomicsaturationdemands morefeaturesandthepresentmethodpresentsamethodbywhichnewfeaturescanbeevaluatedforutilit yasadditionalbiomarkers.

Humansiblingspresentexamplesofphenomicfeaturesbeingproductsofbothnaturea ndnurture.Humanshaveevolvedorbeentrainedtorecognizeresemblancesbetweenrelativescausedby numerousbiomarkers. However, thesesubtleandheritabledifferencesfromcombinationsofbiomarkers (includingovertime) thatmakeeachindividualuniquearedifficulttoexplain and are not yet quantified as phenotypes, some combinations of these may be predictive ofotherphentoypesofinterest.FIG. 5illustratesanexampleoftherelationshipbetweengenetics, environ ment, anderror. Obvioustraitslikehaircolor,eyecolor, orothernear- Mendeliantraitscometomindtodescribedifferencesbetweenindividuals, butthesealonearenotsufficie nttorecognizepeople.However, imagingandcomputationaladvancescannowaccuratelymeasuremore subtleandquantitativebiomarkerdifferences, forexamplethedistancebetweencheekbones, the wrinkling around themouth, theshapeof the nose [43, 44]. Suchphenomic approachescannowpopulateamatrixwiththousandsofsuchbiomarkermeasurementsperindividualfro mRGB camera images. These measurements can be used to predict relationships between individuals inthesamewaykinshipmatrices usegenomicSNPstofindrelationships

inanimals, plantsandhumans [47] andinasimilarwaythathumanbrainscanseetheserelationships [48].Th eseconnections can be important because it is believed that closely related individuals tend to performsimilarly (althoughnotthesame) inmanyfacetssuchaspersonality, athleticability, orhealthchoic es [49,50]. Individualswhogrowupinsimilarenvironmentstendtosharephenotypesaswellsasphenotypi cbiomarkerfeatures, forinstancetimespentinawar

zonewilldecreasechildren′sheight [51] orthatbirthmonthsimpacthumandiseaseprobabilities [52]. Ther efore, phenomicmeasuresincludingapluralityofbiomarkersandphenotypescanbeimportanttocapture notonlygeneticsbuttheenvironmentwhererelativeswereraisedandallowimprovedpredictionsforthati ndividual.Plantbreeders, wholookfrequentlyandcloselyattheirplantprogeny, also often recognize resemblances between progeny of their plant crosses that others willnot, partofthe “breeder′seye” ofsubtlepreviouslyuncharacterizedbiomarkersusedinprogenyselecti on.

The example implementation of the present disclosure can include

methodsleadingtosaturatingthephenomewithbiomarkersandallpossiblephenotypes, and/orde terminingthatthephenomehasbeensaturated.

Ingenomics, linkagedisequilibrium (LD) isagoodmeasurebutalsoadifficultconcept.C ompleteLDmeansadifference (polymorphism) betweentwoDNAbasepairsareperfectlyassociatedacr ossallmeasuredorganisms, ifthevariantatonelocationisknowninasample then so is the other. Each additional locus measured, when not in perfect LD or correlationwithothers, addsfurtherpredictiveinformationintothephenotypeandpotentialoforganis ms.

OnceadditionalmarkersmeasuredareallincompleteLD, thegenomeandpopulationarefullycharacterize d, andnoadditionalvariationcouldbeattributabletothegenome. Inpractice,thisremainsanunachievable idealunlesseverybase pairissequencedineveryindividualinapopulation.Likewise, inphenomics, acorrelationmeasuresimilart oLDbetweenallmeasuredphenotypescouldbemaximized. Inthepresentexample,ifnewbiomarkersaref oundwithcorrelationslessthan1 (orgreaterthan-1) toall previousbiomarkers, thephenomeisnotsaturated.Thissuggestsmorebiomarkersareavailableformeas ureanduseinpredictinganorganismsphenotype. Inpreviousstudies, itappearsthatthatevenwithmanyd ozenvegetationindices extracted from a few RGB spectral bands, and many closely spaced timepoints, correlations between phenomic features remain incomplete and thus more biomarkers are available fordiscoveryanduseinpredictinganorganismsphenotypes.

FIG. 6A illustrates genomic correlations. FIG. 6B illustrates phenomic correlations. Thecorrelationswereperformedon520recombinantinbredlines, wheretheGenomicdatahad11,334SN Ps (polymorphicDNAmarkers) aftercleaning.Phenomicdataincluded896featuresobtainedby32RGBder ivedvegetationindicesover14flightdatesandtwomanagementenvironments (droughtandirrigated).Bo ththegenotypeandphenotypecorrelationsshowninFIGS. 6Aand6Bhavemeasureswithhighandlowcorr elationsandareunsaturated. Thissupportsthat additional biomarkers and variation can still be discovered and exploited for prediction modelsandbiologicaldiscovery.

The study shows that new biomarker measurements have practical applicationsforpredictingattributesofpeople, plants, andanimals. “Saturating” thephenotypeisthere forebeneficial, andtheexampleimplementationcanusehigh- volumetemporalmeasurementsonapopulation′sindividualsacrossmanyenvironments.

### EXAMPLES

The following examples are put forth so as to provide those of ordinary skill in theartwithacompletedisclosureanddescriptionofhowthecompounds, compositions, articles, devicesa nd/ormethodsclaimedhereinaremadeandevaluated, andareintendedtobepurelyexemplary and are not intended to limit the disclosure. Efforts have been made to ensure accuracywith respect to numbers (e.g., amounts, temperature, etc.), but some errors and deviations shouldbeaccountedfor. Unless indicatedotherwise, partsareparts byweight, temperatureisin® Corisatambienttemperature, andpressureisatornearatmospheric.

### Example 1: Temporal field phenomics allows discovery of nature AND nurture,socanthephenomebesaturated?

Anorganism′sphenomeresultsfromexpressionofitsgenome (nature) undercertain environment and management effects (nurture) and interactions between these factors, aswellasmeasurementerror.Forover30 years, DNAsequencingandgenomicstoolsadvancedtowhereiti s nowfeasibletosaturategenomes ofsegregatingindividuals,suchthatpolymorphismsatnearlyanypositioncanbedeterminedfromotherk nownpositions. Thisisduetostructure, linkagedisequilibrium (LD), orlinkageandisapowerful tool for genomic prediction and investigating biologicalphenomena. Incontrast, most phenomicstodatefocusesonautomatingpreviouslyknown “traits” asmeasurableandinterpretablephe notypes;akinto focusing onmeasuring a single DNA marker rather thanmeasuring an entire saturated genome. Viewingphenomicsasa platformfordiscovery,similar togenomics, opensnewmethods forcapturingphenomenainnatureandnurture.Saturatinga phenome wouldmeanthatanindividual's fitness, performance, responses toenvironmentand/orspecific phenotypescould be accurately predictedinuntestedenvironments. To date, experience with phenomic prediction for cumulative, complexphenotypes such as grain yield suggests it's possible to predict organismal performance in untestedenvironments, possibly better than genomic methods despite less advanced tools and data. Factorslimiting to saturating a phenomeare evaluating enough individuals and environments, but moreimportantly, tools and methods to extractor “sequence” more phenomic features. Successfullysaturating phenomes will impact every aspect of scienceandsociety, inbiologicaldisciplines fromgermplasmcurators,physiologists tobreeders, to education, thecourtroomandpolicy.

Achallengeofphenomicsisconnectinganorganism's genotypeor genome, totheir phenotype and their phenome. While genomics has had nearly half a century of research, organismalphenomes,goingbeyondmeasuringafewtargetedphenotypes,remainarelatively newconcept. Intheplantresearchcommunity, this linkstoapplicationsfor societalgrandchallenges,suchas sustainable foodsecurityunderclimatechange.Amoonshot, incontrast, isalargeandaudaciousproject.Twonotable moonshotsingenomicshavebeentosequencethehuman genome, which succeeded beyond measure despite some bumps, and the Arabidopsis 2010project, todeterminethefunctionofallgenesinArabidopsis by 2010, which proved overly- ambitious and infeasible. Regardless of success, moonshots canserveasguidepostsforacommunityofresearcherstowork towards, discoveringnewbarriersalongtheway.Oneclearmoonshotforthephenomicscommunityis tosaturate the phenome.Saturatinga phenomeofanindividual or population would mean that no new measurements of an organism can be made thatcannotalreadybepredictedfromothermeasures. Inotherwords, thedepthofphysiologicalresponse softheorganismacrossanypossibleenvironment, management, orscenario (withinreason) can be reduced to numbersandpredictedatanypointintime.Thisisanaudaciousgoaland requires predicting not only nature (the genome), but nurture (the response of an organism toenvironment and managementconditions) aswellasinteractionsbetweenthetwo.

Controlled environment research seeks to create specific conditions to accuratelyassessorganismalresponsesundervariedconditionsthroughouttime.Computermodelsared evelopingsynthetic plants, in silico, to mimic actual plant growth and responses, with goals ofprediction, Al training, and understanding gaps in physiological knowledge. New software packagescaneasilycreatedozens orhundreds of measurements basedon imaging tools for field, root, orcontrolled environments. These disparate activities are all integrable into a larger goal of saturatingthephenome. Controlledenvironmentsreduce environmental, management, and error noise inphenomic measurements and add a level of precisiontounderstandingthephenome.Controlledenvironments also have the potential capacity to collect more measurements within environmentsthroughfullyautomatedsystemsthatareimpracticalunderfieldconditions. However, highlycontrolled systemscannotscreen the number or diversity of environmentsor the number ofindividuals needed to saturate phenomes, so this paper focuses primarily on field systems.

Sixmajorapproachestophenomicsandhighthroughputphenotyping [00145] Goals and use of high-throughput field phenotyping and phenomics across environments,specifically field phenomics with drones or rovers, can begrouped into six majorapproaches. The first, and most common, is automating existing measurements such as plant height, disease lesions, lodging, and plantpopulationcounts.Whilethishelpsscaletraditional biologicalknowledge to more genotypes,itis likeenvisioninghighperformancecomputingjusttocompletethousands of peoples taxes. A second approach is making new measurements that were previouslyinfeasible or impossible manually, for example plant growth over time or novel spectral signatures. This approach hypothesizes that there are valuable traits thathavenot previously been measured, inviting new insights into biology that could never be gained without novel phenomic tools. A thirdisphenomic selection,viewingphenomicsasa platformtoquantifyandcharacterizebiology, requiringacompleteshiftinphilosophyofhowtoapproacht hesephenomicstoolstomaximizemeasurements.Thisisamoredifficultconcept, butonethatmotivatess aturatingphenomes.Afourth isthe useofdeep learning (oftencalledartificialintelligence) tofindpatternsdirectlyfromimages. The fifth and sixth are applications and use cases of the first four with high interest amongplant and crop scientists. Fifth is the discovery of new physiology, biology and genetics to helpscience and scientists advance basic knowledge in these areas, for which new predictive phenotypesare valuable. Sixthis discovering new traits or signatures for intervention that can be deployed on farms. For instance, detecting disease early to deploy pesticides, or drought signatures for irrigationbeforeyieldislost.

Phenomic selection

Phenomicselectionwasfirstcoined, proposedandexploredbyRincentetal. (2017) using laboratory near infrared reflectance spectroscopy (NIRS) measures of wheat grain andpoplarbark. Incredibly,throughcross- validation, theyshowedthatusingmanyreflectancebandsfromtheseproductscouldaccuratelypredictyi eldsinapopulation. Thiswasvalidateddirectlyorindirectlyinmultipleotherstudies. Most incredibly, Rincent et al. (2017) demonstrated thatphenomic selection approaches couldoutperformgenomicselection, thegoldstandardinpredicting phenotype, andwithlesscost. InthecaseofNIRS, thousands of reflectance bands aretreated similarly to genomic markers in genomic selection; quasi-independent repeatable measuresseparatingindividuals. Infieldstudies, however, evenwiththemostintensemeasurementofkn ownphenotypes (e.g., height,leafangle,floweringtime,etc.) arichenoughdatasetcannotbecreatedtore peatablyseparateandpredicteachgenotype.Furthermore,mosttraditionalphenotypesare endpointmeasures,where interactionswithenvironmentareintegratedovertheorganism′slife.Unoccupiedaerialsystems

(UAS, UAV,drones) haveprovidednewways tocollectmassiveamountsofdataacrosslargenumbersofindividualsandenvironments, extractingnovel features. Yet unless hyperspectral bands are used, challenges remain in collecting enough featurestouseinphenomicprediction.Adaketal. (2022) overcamethishurdlebyusingdensemeasureme nts of temporal features, which was multiplicative in the number of phenomic featuresandallowedearliernear real- timepredictionability.Thisshowedthattemporalphenomicpredictioncanindeedperformsimilarly,perh apsbetterthangenomicprediction.However, evenwiththe largest reported UAS dataset, only 896 phenomic features were extracted compared with11,000genomicmarkers.Phenomicsaturationdemandsmorefeatures.

Phenomic selection and phenomics as a platform

Aculturalshiftthroughoutscienceandsocietyisrequiredtounderstandandjustifyphe nomics′grandchallengeandmoonshot. Inmolecularquantitativegenomics, radicalchanges in application and thought came from shifting goals of genetic mapping, trying to find locicontrollingspecifictraitsvariation,togenomicselection, usingalllociacrossthegenometofind “theb est” individual.Notably,inboth, moregenetic markerswerealwaysseen asbeneficial to characterizemorethoroughlywhatwasoccurringin thegenome.Markernumberswereinitiallylimitedbytechnologyandresources (e.g.RFLPsandSSRs) comp aredwithcurrentfinanciallimitationstoscreenfor SNPs. Inmaizegeneticmapping, thenumberofDNA markers went from190 in 2002, to 1329 in 2006, to 12.2 million in 2022. Despite these advances, the stated Arabidopsis2010 goal of determining the function of every gene in the genome is a little closer. It is somewhatparadoxical that capturing as many phenomic features as possible, without a prioriknowledgeofthe underlying biology is considered by some a fishing expedition. Scientific communities have yettofully considerthebenefits ofsuch “random” phenomic measures. Humans areagood exampleofhowsuchrandomphenomicmeasurescanbeextendedtodiscovernatureandnurtureovertim e.

Human siblings as an example of phenomic features being products of bothnatureandnurture

Humanshaveevolvedorbeentrainedtorecognizeresemblancesbetweenrelatives.

However, when asked what causes resemblance there is usually a loss for words (FIG. 5).Obvious traits likehaircolor, eye color, orothernear-Mendeliantraitscometomind. However, imaging and computational advances can nowaccurately measure more subtle and quantitativedifferences, for example the distance between cheek bones, thewrinklingaround themouth, theshapeofthenose.Suchphenomic approaches couldnow populateamatrixwiththousandsofmeasurementsperindividualfromRGBcameraimages. Thesemeasu rementscanbeusedtopredictrelationshipsbetweenindividualsinthesamewaykinshipmatricesusegen omicSNPs tofind relationships in animals, plants and humans and in a similar way that brains of humans can seetheserelationships. Theseconnectionscan beimportantbecausecloselyrelated individuals canperformsimilarly (althoughnot necessarilythesame) inmanyfacetssuchaspersonality, athleticability, orhealthchoices.Itis alsoknownthatindividuals whogrowupinsimilarenvironmentstendtosharephenotypicfeatures, forinstancetimespentinawarzo newilldecreasechildren′sheightorthatbirth months impact human disease probabilities. Therefore, itis expectedthatphenomic measures to capture not only genetics but the environment where relatives were raised.Plantbreeders, wholookfrequentlyandcloselyattheirplantprogeny,alsooftenrecognizeresemb lancesbetweenprogenyoftheirplantcrossesthatotherswillnot, partofthe “breeder′seye” usedinprogenyselection.

Evaluating phenomic measurement success: how will it be known if thephenomeissaturated?

Ingenomics, linkagedisequilibrium (LD) isagoodmeasurebutalsoamongthemostdiffic ultconcepts.CompleteLDmeansapolymorphismbetweentwoDNAbasepairsareperfectlyassociated, ifthevariantatone locationisknowninasample thensois the other. Eachadditionallocusmeasured, whennotinperfectLDorcorrelationwithothers, addsfurtherpredi ctiveinformation. Onceadditional markers measuredare all incomplete LD, thegenomeandpopulation are fully characterized, and no additional variation could be attributable to the genome. Inpractice, thisremains anunachievableidealunlesseverybasepairissequencedineveryindividual.Likewise,inphenomics,acorr elationmeasuresimilartoLDbetweenallmeasuredphenotypescouldbemaximized.Currently, ifnewphe notypesarefoundwithcorrelationslessthan1 (orgreater than-1), thephenome isnotsaturated. From publishedandpreliminarystudiestodate, itappearsthatevenwithmanydozenvegetationindicesextract edfromafewRGBspectralbands, andmanycloselyspacedtimepoints, correlationsbetweenphenomicfe aturesremainincomplete (FIG. 6). Thissupportsthatadditionalphenotypicvariationcanstillbediscovere dandexploited.

### CONCLUSION

For the phenomics community to mature beyond being seen as simply working toincreasethethroughput ofmeasurements, orreducedtoserviceofgenomics, abroadervisionincludingagrandchallengeandamo onshotareneeded. Theareaofphenomicselectionhasalreadyprovidedtantalizingnewinsightintohow massive amounts of biological phenotypic datacan be used to discover unanticipated biological truths. For phenomics discovery to succeed, similarapproachesneedtobeappliedtoareassuchasgermplasmconservation, ecologyandultimatelyimprovementsinfarmers′fields. Thehistoryand developmentofthefields of genetics, statistics, and even evolution owe a great deal to agricultural improvement research interests, and phenomicscould follow asimilar trajectory. The bottleneck to date, is highvolumetemporalmeasurementsmadeonapopulation′sindividualsacrossmanyenvironments.

### Example 2: Temporal Image Sandwiches Enable Link between Functional

### DataAnalysisandDeepLearningforSingle-PlantCottonSenescence

Summary: Senescenceisahighlyordereddegenerativebiologicalprocessthataffects yield and quality in annuals and perennials. Images from 14 unoccupied aerial system (UAS, UAV,drone) flightscapturedthesenescencewindowacrosstwoexperimentswhilefunctionalprinci palcomponentanalysis (FPCA) effectivelyreducedthedimensionalityoftemporalvisualsenescenceratin gs (VSRs) andtwovegetationindices: RCCandTNDGR.Convolutionalneuralnetworks (CNNs) trainedonte mporallyconcatenated, or “sandwiched,” UASimagesofindividualcottonplants (GossypiumhirsutumL.), allowedsingle-plantanalysis (SPA). Thefirstfunctionalprincipal component scores (FPC1) served as the regression target across six CNN models (M1- M6).ModelperformancewasstrongestforFPC1scoresfromVSR (R2=0.857and0.886forM1andM4), strong for TNDGR (R2=0.743 and 0.745 for M3 and M6), and strong-to-moderate for RCC (R2 =0.619and0.435forM2andM5), withdeeplearningattentionofeachmodelconfirmedbyactivation of plant pixels within saliency maps. Single-plant UAS image analysis across time enabledtranslatableimplementationsofhigh- throughputphenotypingbylinkingdeeplearningwithfunctionaldataanalysis (FDA). Thishasapplications forfundamentalplantbiology, monitoringorchardsorotherspacedplantings,plantbreeding, andgenetic research.

### INTRODUCTION

Senescence: Senescence encompasses the summation of gene-, cell-, tissue-, andorganism- level59changesleadingtodeteriorationofbiologicalfunction.Shuttlingofassimilatesfromvegetativeto 60reproductiveorgansincropplantsisakeyfeatureofend-of-seasonsenescenceasitimpacts harvestindexoffruit, yieldofgrainorseed, nutrientcomposition, andefficiency of nutrient use (Gregersen et al., 2013). Structural changes to senescing cells occur in anorderedmanner, withleafsenescenceundernuclearcontrol (Gan,2003,Yoshida,1962).Plantscientist sfrequentlyinterpretsenescenceasaresponsetostressandfactoritintoselectiondecisions for breeding (Makanza et al., 2018), underscoring senescence as a quantitativeselectionmetricthatcansimultaneouslybeanalyzedasatime-seriesdatamodality.

Althoughitisgrowncommerciallyasanannualcrop, Gossypium (cotton) maintainsani ndeterminategrowthhabitcharacteristicofperennials (ChenandDong,2016).Asopposedtoannuals,wh erenutrientsareallocatedtodevelopingseeds,seasonalsenescenceinperennials is marked by nutrient shuttling to stems or roots, where they are reserved for growth inthenextseason (Wooet al.,2019).G.tomentosumisawildallotetraploidspeciesnativetothe

HawaiianIslands, whereitisreferredtoasMalo (DeJoodeandWendel,1992).SeveralG.tomentosumtraits aredesirableforintrogressionintocultivatedcottonspecies, includingitscharacteristic heat tolerance, resistance to pests and diseases such as fleahoppers, tarnished plantbug, bollworm, and boll rot (Saha et al., 2006), thrips and jassids (Hulse-Kemp et al., 2014), and fordesirable agronomic traits including fiber quality, length, and fineness (Shim et al., 2018, Zhang etal., 2011). In cotton, late- season weather conditions influence senescence timing, with prematuresenescence and late boll maturity potentially conferring reductions in fiber quality and yield (Dongetal., 2006).Leafsenescencecanbe accelerated byextreme highorlowtemperatures, withhightemperaturespromotinganincreaseinchloroplastreactiveoxygensp ecies, thusdamagingthechloroplastandphotosynthesis- associatedproteins, whichultimatelyimpactsphotosyntheticelectrontransfer (Oughametal.,2008).Abi oticfactorsincludingdrought, limitednutrients, oxidativestressviaUV- Birradiation, andbioticfactorssuchasplantpathogensandshadingcaneachinduceuntimelysenescencea loneorincombination (Limetal.,2003). Theagingofmanycropspeciesdemonstratessensitivitytothesour ce-sinkratio.Cottonsenescenceratecanbedelayedbyremoval offruitingbranches (increasingthesource-sinkratiobyremovingsink tissue) andacceleratedbyremovingleaves (decreasingtheratiobyremovingsourcetissue) (Niuetal.,2007, ChenandDong,2016).

High- throughputphenotypinganalysesofsenescence: Despitetheimportanceofsenescenceasatrait, robust evaluationsoflargenumbersofgenotypesand/orgenotype

Xenvironmentcombinationsarecomplicatedtoevaluateacrosstime (temporally) usingrepeatedobserv ationsspanningtheoverallmaturationperiod.Becausethefloweringhabitofcottonisindeterminate, the maturation period can last weeks to months. This “phenotyping bottleneck” forsenescencecanbemitigatedusingunoccupiedaerialsystems (UAS, andTester,2011) formingabasis of field-based high-throughput phenotyping (FHTP). An early application of greenhouse- basedHTPproximalsensingshowedthatdespitecolordistortionorblurring, empiricallydeterminedsenes cencescorescouldbeobtainedfromimagesofAustralianspringwheat (Triticumaestivum) andchickpea (C icerarietinum) withmoderate-to- strongpositivecorrelationstovisualscores (Caietal.,2016).Lyuetal. (2017) reportedacomprehensivegre enhousepipelinethatdissectssenescenceatthesingle- leaflevelusingleaftrackingandprincipalcomponentanalysis (PCA) ofempirically-determined senescence scores to cluster wildtype Arabidopsis and senescence mutants intogroupswithdistincttemporalphenotypes.Visuallyscoringsenescenceintime- seriesUASorthomosaics of maize hybrids, DeSalvio et al. (2022) identified quantitative phenotypic indicatorsofsenescenceprogressionthroughplot- basedtemporalvegetationindices. EachstudydemonstratedHTP (greenhouse-orfield- based) candecryptsenescencequantitativelyusingspectral data. However, further methodological development and throughput of phenomics- basedsenescencecharacterizationisrequiredforplantbiology, breeding,genetics, andcommercialappli cations.

Plantbreedingprogramsdependongenerationaloryearlyrecordingofphenotypictrait s,manyofwhichrequiretime-consumingandlabor- intensivecollectionmethods.Accuratelymappingconnectionsbetweenphenotypeandgenotypeandult imatelysaturatingthephenome (Murrayetal.,2022) requiresmethods thatcanshuttlebroadclassesofdatathroughautomatedprocessingpipelinesrequiringlittlemodification ineachusecase.Beginningwithrawinputssuchasimages,deeplearningmethodsfunctionviaaseriesofno nlinearlayersthattransformtherawinputstoslightlymoreabstractrepresentationswitheachlayer, ultim atelyamplifying “signal” from “noise” (LeCunetal.,2015).For example, earlylayersmaydetectbasicfeatur es such as and leaf pigmentation attributable to infections by different pathogens. Within thedeeplearningclassofmodels, convolutionalneuralnetworks (CNNs) aresuitableforimagerecognition and categorization, learning complex and nonlinear mappings from largeexample datasets (LeCunetal.,1998).CNNsaregenerallycharacterizedbythreetypesofneurallayers: convolutiona I, pooling, andfullyconnectedlayers (Guoetal.,2016).Duringtheforwardstageoftraining, theinputimage, usually anRGB pictureintheformofa3-dimensional tensor (whereXisheight, Yis width, andZisdepth),ispassedthrougheachlayerwherethe currentweightsandbiases withineachlayer areapplied, andtheoutput (apredictioninthe formofa classlabel orregressionoutput) issubsequentlycomparedwiththegroundmeasured “truth” valuetocalculateloss. After eachconvolutional layer, nonlinearity is often introduced using theRectifiedLinear Unit (ReLU) function (Glorot et al., 2011) which also combats the vanishing gradient problem (Pawara etal.,2017).Duringthesecondstageoftraining, backpropagationentailsiterativeapplicationofthechainr uletocalculatethegradientofthelossfunctionwithrespecttoeachparameter,withparametersupdatedb asedonthesecalculations (Guoetal.,2016).Fullyconnectedlayersgenerally employ dropouttoavoidoverfittingby randomly removinga percentage of nodes within the fully connected layer during training and backpropagation (Srivastava et al., 2014, Yoo, 2015,Pawaraetal.,2017).

CNNapplicationsinplantsciencesincludesegmentationofoverlappingfieldplantsin maize (Guo etal.,2023), classificationof soybeanstress (Ghosal etal., 2018), and diseasedetectioninbellpepper,potato, andtomato (Jungetal.,2023), wheat (Nigusetal.,2023), andacross thePlantVillagedataset, whichincludes39classesofplantleaves withvaryingdiseases (Mohantyetal.,2016). UbbensandStavness (2017) demonstratedanearlyapplicatio nofneuralnetworksforleafcounting,classifyingmutants, andplantageusingprimarilytheInternationalPI antPhenotypingNetworkArabidopsisphenotypingdataset (Minervinietal.,2014). Inaregressioncontext, Niuetal. (2024) reportedtheuseofrow-andgrid- leveltemporallyconcatenatedimageswherefourRGBimages,eachwithdimensions32x 32x3,werestackedalong the third dimension (time) to produce 32× 32×12 images (where 12 is calculated by 3 RGBchannelsperimage

X4images). Withthisinputdatastructure, theCNNreportedcottonyieldvalueswithlowermeanabsolute errors (MAE) andhigher R2valuesthanAlexNet, ResNet, andCNN-LTSMmodels. This study highlightedthatinputdatacanberestructuredsuchthattimeseriesinformationisembeddedineachinput unit, therebyreducingthedimensionalityofthedatasetdowntothenumberofindividualplotsorinthecas eofsingle-plantanalysis (SPA), individualplants. No CNN method has yet been reported to enable temporal SPA of senescence in field grownrowcrops.SPAwouldrepresentaparadigmshiftfromwhole- plotanalysisthatiscurrentlypervasiveincropexperiments.MostSPAstudiestodatehavefocusedonindivi dualtreephenotypingandhave beenconductedmanually withoutthe benefitofCNNs.NovelSPAmethodscouldenableincreasedstatisticalpowerwithoutrequiringincreasedl andusage, refiningthedissectionofgenotypeXenvironmentinteractionsatthesingle- plantlevel, andearly-generationselectioninplantbreeding.

Functionaldataanalysisinplantsciences: Functionaldataanalysis (FDA) isastatisticalf rameworkfor theanalysisandtheoryofdatathatvaryoveracontinuum,suchasrepeatedmeasuresofa plantphenotypictrait (e.g., plantheight,diseaseseverity) acrosstime.Functionalprincipalcomponentan alysis (FPCA) enablesdimensionalityreductionandimputationwhensparsityispresent (Wangetal.,2016).FPCAcancapturedominantmodesoftemporalvariation embedded in noisy field data. To model temporal NGRDI vegetation index values of maize growninirrigatedanddroughtconditions,Adaketal. (2024) usedFPCAtopartitionthespectraldatabasedonthemanagementconditionsusingthefirstandsecondfun ctionalprincipalcomponents (FPC1andFPC2) andtosettheFPC1andFPC2valuesasresponsevariablesfor quantitativetraitlocus (QTL) analysis. This pointedtowell- knownQTLsimplicatedinmaizestressresponsesandgrowthregulation. Inthecurrentstudy, theconcepto freducingdimensionalityoftemporal data via FPCA and using FPC1 scores as CNN regression target variables is explored in thecontextoftemporalconcatenationofsingle- plantsenescenceimages, anotherdimensionalityreductiontechnique.Thiscomplementsmethodsprop osedbyNiuetal. (2024) forregressionofcottonyield, howeverhere, theregressiontargetisatemporalphe notype (FPC1senescencescores) insteadofayieldvalue.

The methods proposed here serve to address the growing need for novel analysistechniquesindissectingtheplantphenomeandconductingtargetedplantbiologystudiesaswell asbreeding. Themainobjectivesofthisarticlewereto: 1) performdimensionalityreductionoftime- seriessingle-plantsenescencescoresusingvisualandempirical (vegetationindex- derived) senescencescoringmethods;2) trainCNNswithtemporallyconcatenated (“sandwiched”) single -plantimages to learnmappings frominputfeaturesto predictthe targetvariable

(FPC1scores) ofvisual and empirical senescence scores; and 3) to perform a preliminary statistical analysis of time-seriessenescencedataincottonchromosomesubstitutionlines (CSLs) andchromosomesegmentsubstitutionlines (CSSLs).

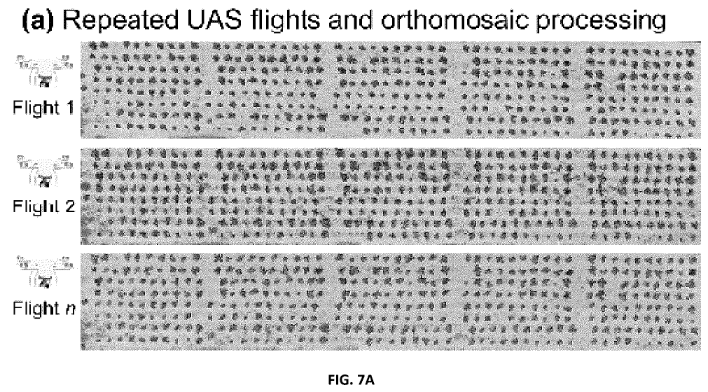

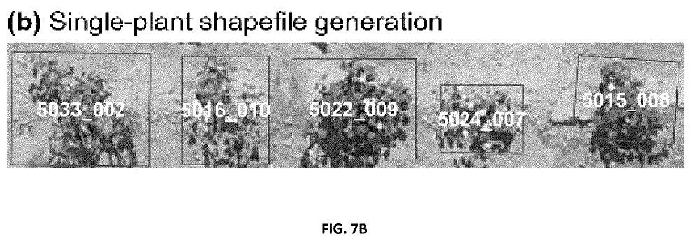

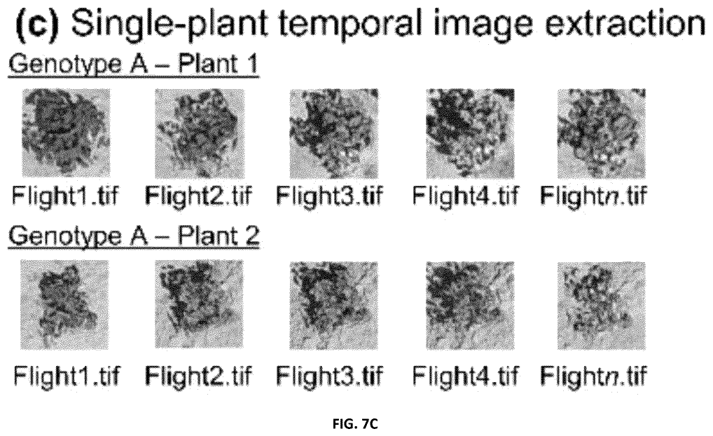

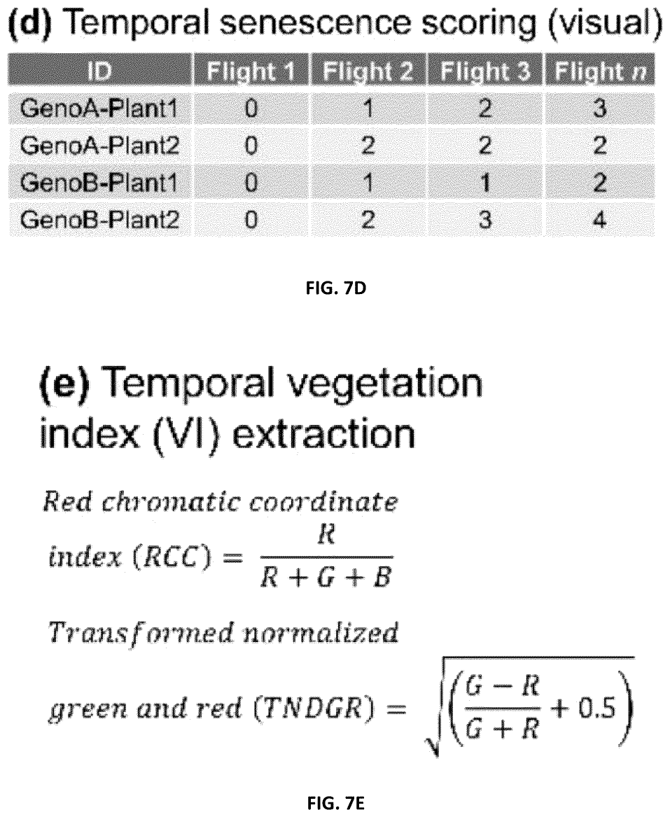

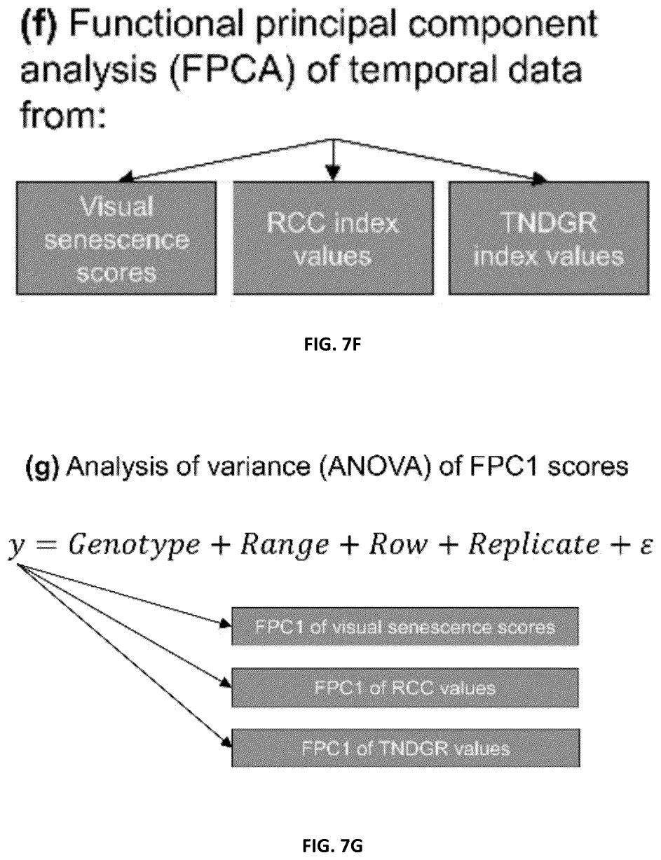

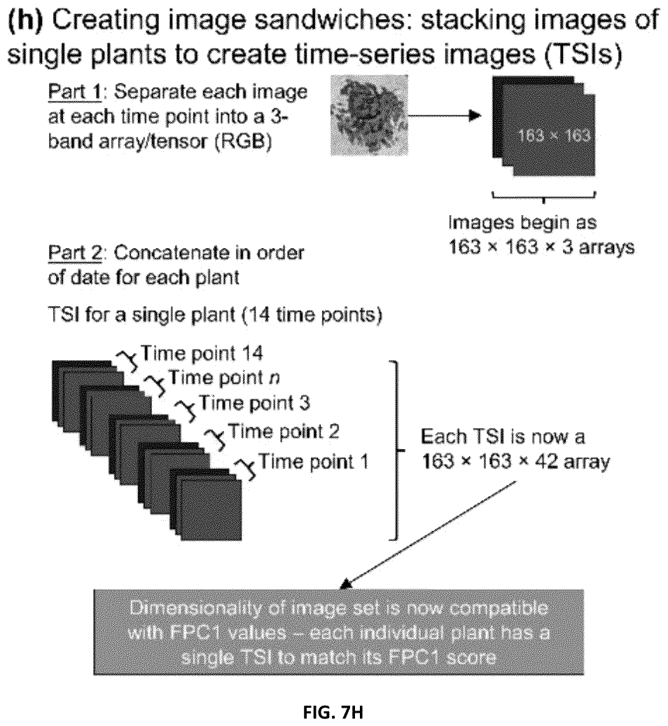

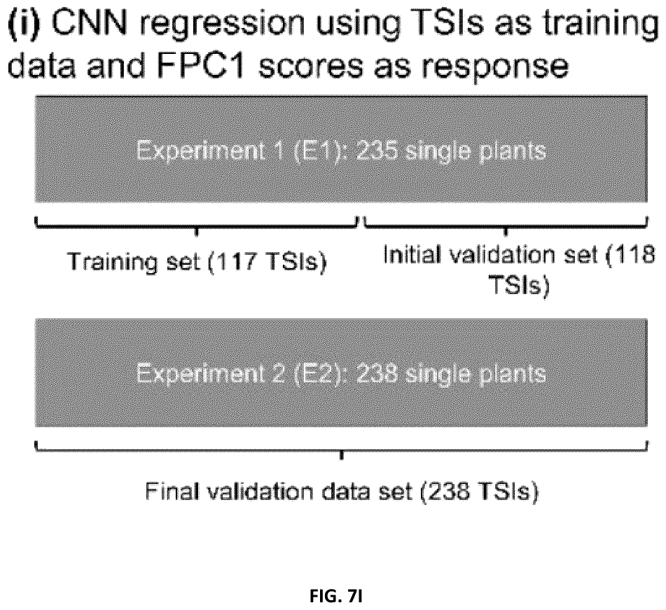

Materials and Methods: FIGS. 7A-71 depict a graphical summary of the methodsemployedinthisarticle. Descriptions ofthemethodsusedarelistedintheorderinwhichtheyappearinthefigure.

Cottongermplasmandexperimentaldesign: Afieldexperimentwas conductedin College Station, TX, between April and September 2023 to evaluate upland cotton (G. hirsutum) BCsbackcross-inbred lines containing small portions of the genome of the wild Hawaiian cotton G.tomentosum (Nutt.exSeem.),including 12CSLs, 35CSSLs, two uplandlines (TexasMarker 1, TM-1), andanon-experimental “filler”.Greenhousegrownthree- weekoldseedlingsof49uniquegenotypes were mechanically space-transplanted into a randomized complete block design (RCBD) with10rows (ca. 200-cm spacing) of10 plants (ca. 180-cm spacing), with outer rows and end hillsservingasnon- experimental “border”, thusyielding48spacedtransplantsineachofthefive blocks. Each genotype had approximately 5 replications with a total of 240 individual plants. Due topoor germination or other environmental causes, 5 plants perished early in the season, leaving 235individualplantsfortime- seriesanalysis.Thisexperiment, hereafterabbreviatedasE1,providedthetraining images fortheCNNregressionmodel.Aconcurrentfieldexperiment (abbreviatedE2) with240individualplantsw asconductedduringthesametimeframeandprovidedunseenvalidationimagesforCNNevaluation.Duet oenvironmentalcausesorpoorgermination,2plantsfrom E2 perished, leaving 238 availablefor analysis across 14 timepoints. Germplasm consistedof27genotypes (replicatedapproximately9times) ofadvancedbackcross- inbredlineswithaG.hirsutum background containing small introgressed portions of the G. mustelinum genome. As thisexperimentwasprimarilyintendedforseedincreasesandinitialfiberqualitycharacterization, itwasn otassessedviaanalysisofvariance (ANOVA) andservedstrictly asvalidationdataforCNNperformance.

High-throughputphenotypingofcottonexperiments: Aftertransplantingseedlings to the field, UAS flights were conducted two or three times each week totaling 46 flightsacrossthegrowingseason,ofwhich14late- seasonflightswereusedtomeasuresenescenceoccurring on: 24,28, and 31 July; 4,8,11,14,16,18,21,24, and 28 August; 1 and 5 Sep. 2023.RGB images were stitched to produce orthomosaics (Methods S1) and single-plant shapefiles werecreatedusingtheUAStoolsRpackage (AndersonandMurray,2020) andadjustedwhereneededma nually (FIG. 7A-7B).

Single-planttemporalimageextraction: Single- plantGeoTIFFswereextractedfromorthomosaicsand wereconvertedtoJPEGs (FIG. 7C). Intotal,235and238imageswereobtainedperflight, respectively, leadin g toatotaldatasetsizeof6,622images (473images perflightx14flights).Generally, theRemSoil ( ) unctionwithinFIELDimageR (Matiasetal.,2020) isadvisabl etolimittheeffectsofsoilonvegetationindexcalculations.However, inspectionofimages after soil removal indicated that senesced planttissue was erroneously being removed andled to large holes in the images, likely due to similarity in color with soil, therefore this function wasnotemployedforthisstudy, andthis limitationisacknowledged. Tomitigatesoil effects,shapefileboundingboxeswereadjustedtocropcloselyaroundthebordersofeachsingleplant.

Temporal senescence scoring of single-plant images (visual): For each plant, 14visualsenescenceratings (VSRs) wereassignedaccordingtoflightdateandrecordedintabular format (6,622 totalsenescencescores). Ascoring system withsix levels wasimplemented, where0=0% senescence (completely green), 1=20%, 2=40%, 3=60%, 4=80%, and 5=100% (completely dead) (FIG. 7D). Examples of plants representing each score are shown in FIGS. 8A-8C.Three temporal phenotypes were observed: senescence progressed toward plant death (FIG. 8A), stay-greenoccurredand vigorwasmaintaineduntiltheendoftheseason (FIG. 8B), orplantspresentedaninitial dropinvigorbutdisplayedresilienceandresurgenceofvigor (FIG. 8C). Thetransientdisplayofintermedia tesenescencestagesledtoanimbalanceddataset, whichwasdominatedprimarily byscoresof 0,1,2, and5, withnotablylessexamplesseenfor 3and4, respectively (Table 1). Due to this, and in keeping with previously published standard deep learningdataaugmentationpractices (Ghosaletal.,2018), eachimageunderwentahorizontalinversiona ndthreerotations (clockwise) of90°, 180°, and270°. Thetotaldatasetsizeconsistedof33,110images (6,622initialimages+6,622×4augment ationmethods) (Table1).

Temporal vegetation index (VI) extraction: Using the fieldIndex ( ) function withinFIELDimageR,vegetationindices (VIs) werecalculatedforeachplantateachtimepoint (FIG. 7E).Toi dentifypotentialcandidatesforempiricalsenescencescoringusingVIs,eachindexwassequentiallyasses sedforitsPearsoncorrelationcoefficientwithvisualscoresusingthecor ( ) unctioninR.Twowereselected (RCCandTNDGR) andtherestarenotfurtherdescribed.

Functional principal component analysis (FPCA) of temporal data and analysis of variance (ANOVA) ofFPC1scores. The fdapaceR package (Wang et al., 2016, Chenetal., 2017) was used to performboth FPCA and prediction/imputation of values occurring during the senescence time grid for

VSR, RCCindexvalues, andTNDGRindexvalues (FIG. 7F). TheFPCA ( ) unctionwithinfdapaceinvolvessolvin gtheintegralFPCAexpansionlistedbelow (Karhunen,1946, Loève,1946):

$${X}_{i} ( t ) = μ ( t ) + {\sum }_{k = 1}^{00} ( {}_{k} {∅}_{k} ( t ) ; Equation 1 , \qquad Eq . 1$$

Here,u (t) representsthemeanfunctionandindicatestheaveragesenescenceprogres sionateachtimepointt (i.e., thedateofeachdroneflight); (kindicatesthefunctionalprincipalcomponents coresforeachfunction;Øk (t) representtheeigenfunctionsofkfunctionalprincipalcomponents.Toimput etemporalvalueswithinthesenescencetimegridforVSRs,RCC, andTNDGRindexvalues, thepredict ( ) unc tionwithinfdapacewasused.FPCAwasperformedunder two frameworks. The first framework sought to enable analysis of variance (ANOVA, FIG. 7G) oftemporalsenescence.

Inthiscase, thefirstfunctionalprincipalcomponentscores (FPC1) from analyzing E1 data were used as the response variable. This framework used data from only E1, as30% ofplantswithinE2hadtobereplacedwithgreenhousebackupsseveralweeksaftertransplantingd uetoenvironmentalcauses. Therefore, E2temporalscoreswerenotevaluatedusingANOVA.Sincevarianc edecompositionusingANOVAaimstopartitiongenetic, environmental, and other effects from unexplainedvariation, only data from E1 was analyzedinthiscapacity.

ThesecondframeworkwasintendedtopreparetargetvariablesforCNNregression.P ooledsenescencedatafromE1andE2wereassessedwithFPCA.FPC1scoresfromFPCA of combined E1 and E2 data were stored for CNN training and evaluation. The primary modeoftemporalsenescence progression (VSRs,RCC, orTNDGR) foreachplantwascapturedbyFPC1and later served as the CNN target variable. The two FPCA frameworks produce unitless values foreach eigenfunction (FPC1, FPC2, etc.), thus if E1 and E2 data were not pooled to obtain regressiontargetvalues, itis possiblethattheranges ofFPC1valuescouldhavedifferedbetweenthetwoexperiments (i.e.,FPC1valuesof10inE1andE2mayno tconferthesametemporal phenotype).

To estimate genetic and field spatial variance components of FPC1 values for E1VSRs, RCC, and TNDGR index values, ANOVA was performed via the Ime4 R package (Bates et al.,2014) usingEq.2:

$${Y}_{ijkl} = μ + {Genotype}_{i} + {Range}_{j} + {Row}_{k} + {Replicate}_{l} + {E}_{ijkl} ; \\ Equation 2 , \qquad Eq . 2$$

Yis a vector of length 235 indicating each FPC1 value for observations of the ithgenotype, inthejth range, kth row, andIth replication;uindicatestheexperimentalmean;Genotypeindicatestheeffectoftheith genotype, withi=1,2, . . . 49,whereGenotype ei [symbol] N (0, [symbol] 2); Range indicates the effect of the jth range, with j=1,2, . . . ,40, where Range j [symbol] N (0, [symbol] 2); Row indicates the effect of the kth row, with k=1,2, . . . ,6, where Row k [symbol] N (Q, [symbol] 2); Replicate indicates the effect of the Ith replication, with I=1,2, . . . ,5, where

Replicate 1 [symbol] N (0, a [symbol] ); and Eijki indicates the residual error term that accounts for unexplained variability given by a2 such that E [symbol] N (0, a2).

E ijkl E

Repeatability was calculated according to Eq. 3:

$$Repeatability = \frac{: {i}_{2}^{2}}{{:}_{i}^{2} + \frac{{>}_{?}}{l}} ; Equation 3 , \qquad Eq . 3$$

Here, a2 and a2 indicate the variance components of the effects of genotype and i E the error term, respectively, and lis the number of replicates. In a minority of cases, replicatesdeviated from 5 due to seedling shortages or replacements of seedlings due to environmentalconditions.Creating imagesandwiches-stacking imagesofsingleplants tocreatetime- seriesimages (TSIs)

ImageswereimportedintoPython3.11.5usingiio.imread ( ) romimageioandresizedt o163×163x3witht.image.resize ( ) romTensorFlow, where163is theaverageimagesize (XY dimensions) and 3indicates three RGBcolorchannels. Tocreate time-series images (TSIs) or “image sandwiches,” single-plant images were temporally concatenated using np.concatenate ( ) fromNumPy) along thethirddimensionbyflight date (FIG. 7H).Eachflightaddedthreelayers, resultinginTSIs of 163x 163x 42pixels (3channels X14flights).EachCNNexperiencedeachplantasatemporallystackedimage,withtheFPC1scoreas theregressiontarget. Thedataset′soriginal dimensionality changed from 6,622 images of 163×163 × 3 (combined E1 and E2) to 473TSIsof 163x 163x 42pixels,corresponding to thenumber ofsingle plantsacrossE1andE2.

Post-augmentation, the total was 2,365 TSIs (473 initial+1,892 augmented).

CNNregressionusingTSIsastrainingdataandFPC1scoresasresponsevariables: Inspi redbyZingarettietal. (2020),hyperparameteroptimizationwasundertakenusingtheOptunaPythonpac kage (Akibaetal.,2019) foreachofthethreeregressionscenarios: usingTSIsto regressFPC1scoresof1) VSRs, 2) RCC, and3) TNDGRvalues.Optunaenables the user tocreatea “study,” wherebyanobjectivefunctionisdefinedwithaparameterspacethroughwhichOptun asystematicallysearchestoachieveoptimalperformanceatminimizing (inthiscase) ormaximizing atargetobjectivevalueacrossseveral “trials, “setat250inthisstudy.Eachtrial wasallowedtorunfor50epochs.Here, thestudysoughttofindthehyperparametersthatproducedthelow estmeansquarederror (MSE).MSEwasminimizedbecauseitisdifferentiable (itsderivativecanbecalculat ed), whichenablesasmoothcurvethatgradientdescentcantraveltofind the smallest error. Searchable hyperparameters are summarized in Table 2 and further detailsareprovidedinMethodsS4.Sixmodelsunderwenthyperparameteroptimization, twoeachforVSR s, RCC, and TNDGR index values, with the only difference between them being the specification of the first dense layer always being set to ‘relu’ (M1-M3) or being searchable by Optuna (M4- 6).ModelnamesandassociatedtraitsareoutlinedinTable3.

Model training and evaluation: Model development used TSIs from E1, whereasE2 served as an unseen validation set to avoid overfitting and assess general usability for differentcottongermplasm. Thesame50% ofTSIsfromE1wereusedfortrainingandOptunahyperpara meter optimization across six CNN models (FIG. 71), while the remaining 50% of E1 TSIsservedasaninitialvalidationset.Postaugmentation, thetrainingsettotaled585TSIs (117initial +468augmented) andthevalidationset590TSIs (118initial+472augmented),bringingE1′stotalto1,175TS Is.SettingtherandomizationseedensuredconsistentTSIsforallsixOptunastudies,enablingdirectmodelc omparisons.OptimalhyperparameterswerethenusedforCNNregressionon the full E2 validation set. Final evaluation used 80% of the 1,175 TSIs from E1 for training and all1,190 TSIs from E2 (238 initial+952 augmented) for validation. In each replication, 940 TSIs from E1servedas training (0.8 ×1,175=940), witharandomizationseedspecifiedforrepeatablesplits.Metricssavedforeachepochincl udedloss, validationloss, MAE, andvalidationMAE.RandR2werecalculatedusingR′scor ( ) unction (Pears oncorrelation) toassesscorrelationbetweenactualandpredictedFPC1values.MSE,RMSE, andMAPEwer ecalculatedusingtheMetricsRpackage.

Hyperparameter importances were determined with functional ANOVA (fANOVA) using theplot_param_importances ( ) unction.

ModelswerealsoassessedthroughANOVAofFPC1scoresoutputbythetopperforming CNNsforeachsenescencescoringmetrictodeterminevariancecomponents (Eq.2) and repeatability of predictedFPC1values (Eq. 3).Usingthe optimal hyperparameters determinedbyOptuna,CNNregressionofE1FPC1valueswas performedusing thesametrain/testsplitthatwasusedforthehyperparameteroptimizationtrials, wherea50/50train/test splitwasenactedwithaninternal20% validationset.Afterperforming25replicationsofCNNregressionusi ngarandom 50/50 train/test split within each replication, predicted FPC1 values for each E1 plant wereaveraged, producing a vector of 235 FPC1 values that were subsequently assessed through ANOVA.

Modelexplainability: InkeepingwiththeprecedentsetbyGhosaletal. (2018) for plant stress phenotyping and initially by Erhan et al. (2009) and Simonyan et al. (2013), saliencymapsweregeneratedforeachmodelusingarepresentativeTSIfromastay- greenandrapidsenescenceplant.

RESULTS

Correlations between vegetation indices and visual senescence ratings: In

total, 34vegetationindices (VIs) wereassessedfortheircorrelationswithVSRs (FIG. 9A) usingallavailabled atafromE1andE2,withformulasforeachVIpresented. Thehighestpositively- correlatedindexwithmanualsenescencescoreswastheredchromaticcoordinateindex (RCC) (Woebbec ke et al., 1995) with R=0.75 (FIG. 9B). However, the score with the highest magnitudeof correlation, though negative, was the transformed normalized green 390 and red index (TNDGR) (Tucker,1979) withR=−0.81 (FIG. 9C).

Functionalprincipalcomponentanalysis: FPCAwasperformedtosynthesizeasinglev aluefrom14temporalmeasuresforeachplantundertwoframeworks (MaterialsandMethods).WhenFP CAwasperformedusingdatafromE1only, FPC1andFPC2explained89.1% and7.3% forvisualscores,83.5% and10.1% forRCC, and86.1% and8.9% forTNDGR,respectively.ForVSRsusingpooleddatafromE1 andE2,FPC1explained90.2% ofthetemporalvariation (FIG. 10A),withFPC1explaining83.5% (FIG. 10B) an d 86.9% (FIG. 10C) ofthetemporalvariation for RCC and TNDGR, respectively. In the VSR functional principal component plot, manualinspectionofthe14imagesassociatedwitheachpoint (whichdenotesasingleplant) indicatedtha tgenerally,plantswithanFPC1scoreexceeding10couldbeclassifiedashavinga″rapidsenescence” phen otype (FIG. 8A;FIG. 10A), whilethosewithFPC1scoresbelow10correspondedto″stay- green “orresilientphenotypesnotedinFIG.8Band8C.Adistinctclusterofplantsexhibitingrapidsenescen ceisvisibleontherightsideoftheVSR FPCAplot (FIG. 10A).Clusteringwaslesspronouncedfor RCC (FIG. 10B), butTNDGRdisplayedmoderatepartitioningoftemporalphenotypesontheplot (FIG. 10C).

TemporalsenescencetrajectoriesofsingleplantsaspredictedbyFPCA: Temporal trajectoriesofpredictedFPCAscores forVSRsofplants withinE1 (FIG. 11A) revealedasimilar pattern of separation as the FPCA plots (FIG. 10A). Rapidly senescing single plants displayeda prominent rise in senescence between 110 and 130 days after transplanting (DAT). Six genotypesfromE1werechosentoillustratepotentialgenotypicdifferencesintemporalsenescence (FIG. 11B), in which four of five replicates within genotypes 5002, 5012, and 5021 underwent rapidsenescencewhileallreplicatesfromgenotypes5011,5036, and5038demonstratedvaryingdegrees ofstay-green.

Analysisofvariance (ANOVA) oftemporalsenescencephenotypeswithFPC1scores: ANOVA is an objective way to compare the consistency and precision of different measuresbetween replicates. Results of ANOVA with Eq. 2 (E1 data only) indicated the highest percentage ofvariationcapturedbygenotype (36.8%) wasproducedbysettingFPC1scoresofTNDGRastheresponse (FIG. 12C), followedbyFPC1scores ofVSRs (31.4%, FIG. 12A) andRCC (29.9%, FIG. 12B).RCCreportedthe highestdegreeoffieldspatialvariation, with21.1% ofvariabilityinFPC1 scoresattributabletoroweffectsand9.6% attributabletoreplicateeffects.Visualscoresdemonstratedt hehighestdegreeofunexplainederrorvariationat55.3%. Repeatability,ascalculatedbyEq.3,washighest forTNDGR (0.80),whileR2washighestinRCC (0.61).

Variance decomposition of FPC1 values from the top-performing CNN regressionmodels (regressingE1dataonly) withineachsenescencemeasureindicatedthatgenotypicvar iancewascapturedinagreaterproportionforvisualscoresandRCC,improvingoverANOVAresults from actual FPC1 scores to 34.5% (FIG. 12A), 36.2% (FIG. 12B), respectively, while staying at36.8% (FIG. 12C) forTNDGR.RepeatabilityusingCNNmodelsimproved2.9% forvisualscores, remained constantforRCC, anddecreased2.1% forTNDGRwhileR 2increased2.4% forvisualscoresanddecreased 10.5% and9.1% forRCCandTNDGR,respectively.

Hyperparameteroptimization: ThehyperparametersempiricallydeterminedbyOpt una to perform best for regression of each temporal senescence data set are presented herein,whereCNNmodelstructuresummariesdetailing theoutputshapesandparameternumbersateachlayer arealso detailed. ReLU was chosenas theactivation function for all models except M4, where hyperbolic tangent was selected. Except for M3 and M6 in TNDGR, the learning rate exertedthehighestvariableimportancebasedonfunctionalANOVAresults (FIG. 13A), explainingamaxim umof94% ofvariationinM1 (visualscore).Acrossthe250trials,resultsofeachtrial′sperformanceatattem ptingtominimizetheMSE arereported, withlocalminimaindicatedbyapink/redlinethatprogressesstepwiseasanewminimumisf ound (FIG. 13B).

CNNregressionmodeltrainingmetrics: Meanvaluesfrom25replicationsofevaluati ng M1-6 with unseen TSIs (the entirety of the E2 TSI set) are reported across 50 epochs oftraining within each replication withloss (FIG. 14A) and MAE (FIG. 14B). Lossand MAEscalesaredifferent for each of the threetemporalsenescencemetricsas noted by thedifferencesin Y-axisvalueranges.

CNNregressionmodelperformance: PerformancemetricsforallsixCNNregressionm odels (M1-6) across25replicationsarepresentedinTable4.

Allmetricsrefertoregressionresultsfromunseenvalidationimages (theentireE2dataset).RandR2wereassessedusing the cor ( ) function in R, while root mean squared error (RMSE), mean absolute error

(MAE), meansquarederror (MSE), andmeanabsolutepercentageerror (MAPE) werecalculatedusingact ual and predicted values within respective functions from the Metrics R package. FPC1 values ofVSRswerepredictedbyM1withanR2valueof0.857andameanabsolute percentageerror (MAPE) of1.12%, whileM4performedthestrongestofallmodelswithan R2of0.886.ModelperformanceforRCCwasstrongforM2 (R2=0.619) andmoderateforM5 (R2=0.435), bothwith large MAPE values. Performance for TNDGR was strong with both M3 and M6 exceeding R2=0.74.

Model explainability: Examination of activation maps from the stay-green (FIG. 15A) andrapidsenescence (FIG. 15B) temporalphenotypesfromM4revealedthatthehighestactivationre gionwascenteredonthecottonplantinbothcases.

Thisindicatesthemodelwasrelyingonplantpixelswhenoutputtingregressiontargetvaluesacrosstheenti reTSI.Regionsoflesseryetstill noteworthyactivationwereobservedattheedgesofmany oftheTSIs forsaliencymapsgeneratedfromM2/5andM3/6,potentiallyindicatingheightenedsensitivityofVIstolat e-seasonweedpressurethatoccurredinbothE1andE2 (FIG. 18-21).Additionalexamplesofactivation maps are shown in FIGS. 16A-16B, which show activation maps presented for M1 (visualsenescencescores), FIGS. 17A- 17B, whichshowactivationmapspresentedforM4 (visualsenescence scores), FIGS. 18A-18B which show activation maps presented for M2 (RCC); FIGS. 19A- 19BwhichshowactivationmapspresentedforM5 (RCC);FIGS. 20A-20Bwhichshowactivationmaps presented for M3 (TNDGR); and FIGS. 21A-21B which show activation maps presented for M6 (TNDGR).

### DISCUSSION

Leaf senescence in higher plants such as cotton (Gossypium hirsutum L.) entailscommunicationbetweenpathwaysinvolvedinenvironmentalsensing, hormones, andcircadia nrhythms

(GanandAmasino,1997,Limetal.,2007,Leeetal.,2021).Senescenceisinherentlyafunctionaltraitsince itprogressesalongacontinuum, thus, theamountofdatacollectedforindividuals in a senescence study depends on the time of the researcher and the instrumentation throughput.Elucidatingmechanismsunderlyingsenescencehasagronomicandbiologicalimplicationsfo ramyriadofcrops, particularlyduetoitsimplicationsfornutrientshuttlingfromsourcetosinktissue, withni trogenbeingthepredominantlyremobilizedelement (Gregersen,2011,Gregersenetal.,2013,Havéetal., 2017).Identificationofcropsof anytypewhosesenescencetrajectoriesarefine- tunedtoauniquegrowingenvironmentrequiresselectionprinciplesrootedinthetemporaltrajectoryofs enescence. Thisstudydemonstratedthatfield-based high-throughput phenotyping (FHTP) of spaced single plants using unoccupied aerial systems (UAS) canprovidetime-seriesspectral datathatcanbepairedwithfunctionaldataanalysis (FDA) techniquessuchasfunctionalprincipalcompone ntanalysis (FPCA) toimprovebiologicalsignals (geneticvarianceandrepeatability) whilereducingthedim ensionalityoftime-seriesdata (FIGS. 10A-10C).Thisapproachsimplifies decisions for futureexperimentationorcanenable fine- tuningofselectionsforenvironmentallyadaptedmaterial.Decodingsenescencehasyieldedsuccessinbas ic biology, revealing the tight regulation of the senescent transcriptome (Woo et al., 2016), theinterplay between plant hormones such as abscisic acid, senescence, and drought resistance (Zhaoetal.,2016), andtheactivityoftime- evolvinggeneregulationatpresenescentstages (Kimetal.,2018).

Linkingfunctionaldataanalysiswithdeeplearning: Regression- baseddeeplearningapproachesenablethepossibilityofmappingimagefeaturestocontinuousnumericd atasuchas plantcounting (Ribera etal.,2017), leafcounting (Xieetal., 2023), andplantage (Ubbensand Stavness, 2017). However, in the case of most regression tasks, models map features to outputvaluesatfixedpointsintime.Linkingfunctionaltraitssuchassenescence progressionrequiresinputting data into a CNN in a manner that embeds the temporal dimension, which was achieved inthis study by concatenating 3-channel RGB images of single plants along the third dimension (time) to create tensors withmore than3 channels (“imagesandwiches ”). With this method, the data setfrom the standpoint of input images was reduced from the number of UAS flights X the number ofsingleplantsdowntothenumberofsingleplantswhileholding theamountofspectraldataconstant.

FPCAoftime-seriessenescenceprogressioneffectivelyreducedthedimensionality of temporal senescence data, with the first principal component (FPC1) explainingbetween 83.5% (RCC, FIG. 10B) to 90.2% (VSR, FIG. 10A) of temporal variation for combined data fromE1andE2. VariancedecompositionoftheFPC1scoresby analysisofvariance (ANOVA) forVSRandindexvaluesforRCCandTNDGRrevealedthatbetween 29.9% to36.8% oftemporalvariationwasattributabletogenotype (FIGS. 12A- 12C).Importantly, thisindicatesthattheFPC1values carry genotypelevel variation through the dimension reduction process. In addition, ANOVAof E1 FPC1 values output by CNN regression indicated that genetic variance partitioning was higherwhilerepeatabilityvaluesimproved (VSR,FIG. 12A) orremainedconstant (RCC,FIG. 12B).FPCAreve aleddistinctmodesofsenescencetrajectoriesamongindividualplants (FIG. 11A) andindicatedthatdiffere ncesinsenescencetrajectoriespotentiallyexistamongthechromosomesubstitutionlines (CSLs) andchro mosomesegmentsubstitutionlines (CSSLs) (FIG. 11B).FPCAalsoreducesthecurseofdimensionality, the” l argepsmallnproblem “,wherethenumberofpredictors (p) exceeds thenumberofsamplesinastudy (n), posingproblems forconventionalstatisticalanalysis.

CNNregressionmodelperformance: Learningrateexertedoutsizedeffectsonperform anceofmodelsM1,M2,M4, andM5butscarcelyaffectedtheTNDGRmodels (M3andM6,FIG. 13A).Models M1-M3andM4- M6exploredthesamehyperparametersduringoptimization (Table2) exceptinthecaseofM1- M3,wheretheactivationfunctionforthefirstoftwo dense layers was always set to ReLU. This had a minimal effect on regression of FPC1 scores forVSRsandTNDGRbutaffectedRCC,wherethe R2ofM2was ˜42.5% higherthanM5 (0.619vs. 0.434). An inherent limitation of this approach is that initializing the hyperparameter search withdifferent starting conditions and a limited number of trials reduces the likelihood that two Optunastudies converge on the same hyperparameters. However, the high computational cost of runningeachofthesixmodelsinthisstudylimitedthetrialsto250.

TherobustR2valuesforunseenvalidationTSIs, especiallyforVSRFPC1values (0.857for M1,0.886forM4) demonstratedthatbothmodelseffectivelymappedsenescence- relatedfeaturesinthetemporallyconcatenatedTSIs.ThisrevealedCNNscanmapimagefeaturestoregres siontargetswithembeddedtemporalinformation, therebyuncoveringaspectsoftheorganism′slifehisto ryasopposedtoinformation aboutonetimepoint.SaliencymapsforM1/M4confirmed this (FIGS. 16- 17). Despite strong correlations (RCC, R=0.75, TNDGR, R=−0.81) withvisualsenescenceratings (FIG. 9B-9C) andstrong (R2=0.619,743, and0.745forM2,M3, andM6) ormoderateR2values (R2 =0.435forM5),somemapsshowedinaccurateactivationareas outsideplantpixels, indicatingsensitivitytoweedpressureorlightingdifferences.However,M2/M5 (RCC) andM3/M6 (TNDGR) saliencymapsmostlyactivatedcorrectlyattheplantcenter (FIGS. 18-21). The lower performance of these models vs. M1/M4 may be due to the larger scale ofFPC1 values for visual ratings. Subtle numeric differences for RCC and TNDGR mapped to significantsenescencetrajectorydifferences.ThiswasevidentintheclusteringpatternsobservedinFPCA ofRCCandTNDGR (FIG. 10B- 10C) ascomparedtovisualscores (FIG. 10A).ModerateclusteringinTNDGR (FIG. 10C) mayexplainitshigher performanceinM3/6duetogreaterseparationinFPC1valuesforrapidsenescencevs.stay- greenphenotypescomparedtoRCC.Overall, theVI- basedmodelsdemonstratetheirpotentialasempiricalreplacementsforVSRs.

Developingconnectionstothephenome: Thestudyshowsthattherearenovelheritabl ebiomarkersthatcanbedetectablebydeeplearningovertime, andnotpreviouslycapturedbyalargesetof existingbiomarkers. Thepresentdisclosurecontemplatesextractingadditional novel biomarkers detected by deep learning (e.g., in the saliency maps of FIGS. 15A-21B. The novel biomarkers can be then added to the set of all biomarkers to saturate the phenome withalargernumberofnovelbiomarkersthatcanbepredictiveofphenotypesofinterestusingcomposites coresfromthephenomicbiomarkers.

### Example 3: Deep learning-based high-throughput phenotype of Maize

(ZeamaysL.) tasselingfromUASimageryacrossenvironments

Floweringtimeisacriticalphenologicaltraitinmaize (ZeamaysL.) breedingprograms.T raditionalmeasurementsforassessingfloweringtimeinvolvesemi- subjectiveandlaborintensivemanualobservation, limitingthescaleandefficiencyofgeneticsandbreedi ngimprovement.Leveragingunoccupiedaerialsystem (UAS,alsoknownasUAVsordrones) technology coupled with convolutional neural networks (CNNs) presents a promising approach forhigh- throughput phenotyping of tasseling in maize. Most CNN image analysis is overly complicatedfor simple tasks relevant to plant scientists. Here a methodology for extracting tasseling from RGBimageryusingaCNN-basedapproachwasappliedto220hybrids and30testlinesgrownineightdiverseenvironments (WisconsinandTexas,U.S.A.) thenvalidatedthroug hanunrelatedsetofhybrids.Overallaccuraciesof. 946,. 911,.985, and.988wereobtainedforclassifyingmaizeimageswithorwithouttasselsfromCollegeStatio n,TXin2020;CollegeStation, TXin2021;Arlington, Wlin2021;andMadison, Wlin2021respectively.Byem ployingdeeplearning techniques, largervolumesofphenotypicdatacanbeprocessedenablinghigh-throughputphenotyping in breeding programs. Although large datasets are required to train CNN models, theproposedmethodologyprioritizessimplicityincomputationalarchitecturewhilemaintainingeffectiv enessinidentifyingfloweredmaizeacrossdiversegenotypesandenvironments.

### INTRODUCTION

Achievingcropimprovement,includinghigheryields, hingesoneffectivelymeasuring and managing various phenotypic traits that collectively contribute to yield, quality, andprofitability.Field- basedmanualphenotypingfacesscalabilityandtimeconstraints, especiallywhentraitsofinterestareonl ydetectableduringcriticalbiologicalwindows (Aasenetal.2020).

Modern breeding practices integrate high throughput phenotyping and genotypic data to establishcomprehensivegenomeandphenome- basedanalyses (Araus&Cairns,2014, Rincentetal.2018,Zhu et al. 2021). Whilegenomic automationadvancementshaveaccelerated beyond Moore's law (PolandandRife2012,Delsenyetal2010), twomajorautomationbottleneckspersistinphenomics: ext ractingphenomicmeasurementsandphenotypesfromimagesandefficientlyanalyzingthevastvolumeof phenotypicdataacquired (Furbank&Tester 2011, Minervinietal.

2015,Songetal.2021).Detectingphenotypicvariationinnear-real- timeandatscaleposeschallengeswithoutautomatedapproaches,giventhetime- consumingandpotentiallyerror-pronenature of manual methods. Various methods have attempted to address these challenges but oftendonotmeetthelevelsofthroughputrequiredforlargeplantbreedingprogramsandcommercialbree dingoperations (Lu etal.2017,Shaoetal.2021,A&Sangeetha,2021,Murciaetal.2021).

Maize (ZeamaysL.) isamonoeciousplantwithtasselscontainingthemaleanthers, which produce pollen, at the apex. Maize exhibits considerable variation in flowering timeamongbothinbredlines (Buckleretal.2009) andhybridsacrossenvironments (Rattalinoetal. 2011),resultingingermplasmwithvariationinfloweringinitiation- relatedtraitssuchasthedurationofthegrain-fill period (Daynardetal.1971).Femaleflowering (i.e.silking) ishighlycorrelated with male flowering (Izzam et al. 2017). Flowering time (both female and male) serves asacrucialindicatorofthetransitionfromvegetativetoreproductivegrowth, withsynchronousfloweringi nhybridproductionfieldsessentialformaximizingyield (Workuetal.2016,Cárcovaetal.2000,Baumetal.2 019).Ensuringappropriatefloweringtimeforavarietywithinitsenvironmentofproductionisalsocriticalf oroptimizinghybridseedproductionusing inbredlines, whichcanbedifficultwherephotoperiodassociatedvariationoccurs (Adaketal.2021b).Conv entionalmanualscoringofmalefloweringrequiresphysicallywalkingthefieldeveryday or every few days (with revisit time directly impacting accuracy) and manually estimating when50% ofplants ina plotshowanthers (anthesis) orsilks (silking) (Andersenetal.2005, Mace etal. 2013, Khanetal.2022).Transitioningfrommanualscoringoffloweringinpopulationstomeasurementbyu noccupiedaerialsystem (UAS, alsoknownasUAVsordrones) flightscoulddramaticallyreducelaborandtime.UASplatformscanreadilyc apturehigh- qualityimages, includingtasselinitiation,facilitatingautomateddetectionofmaizetasselinginbreedingfi elds (Kurtulmuş & Kavdir, 2014, Lu et al. 2016, Karami et al. 2021). While tassel initiation and anthesisarenotidentical, theyarehighlycorrelated, especiallyinmaterial withlargeflowering windows (Warrington&Kanemasu,1983), suchasinexoticintrogressionandpre-breedingprograms.

Incorporating artificial intelligence (AI) approaches, specifically deep learning (DL) applications, in plant breeding presents a promising approach to address the volume of unanalyzedoruninterpretedphenotypicdata (Ubbens&Stavness,2017,Naminetal.

2018).PreviousDLapplicationsinautomatedphenotypinghaveprimarilybeenlimitedtocontrolledenvir onmentorground-based field image acquisitions, which are often stationary, manually operated, or otherwiseimpractical for adoption at scales needed in genetic and breeding studies (Ye et al. 2012 Mirnezamietal.2021,Shaoetal.2023).Studiesemployingdeeplearningtechniqueshaveshownpromisei nautomatingphenotypingtaskssuchasmaizetasselmorphologyanddevelopment (Yuetal.2022,Zhang etal.2023), tassel counts (Luetal.2017, Luetal. 2020,Zanetal.

2020), andtheeffectoftasselsonleafareaindexestimationusingvegetationindices (Shaoetal.2023).How ever, thesemethodsareconstrainedbyeithertheirdestructiveornon- scalablemethodologyordatasetstothethousandsofdiverseplotsscreenedinfieldbreedingandgenetics programs (Alzadjalietal. 2021).

Recentadvancementsindeeplearninghaveexpandedthescopeofautomatedphenot yping by applying modern techniques to existing datasets. These techniques include the useofk- meansalgorithms, which are unsupervisedlearning methods forclustering dataintoKgroupsbasedonsimilarity (Kumaretal.2021).Similarly,K- nearestneighbor (KNN) algorithms, whichclassifyobjectsbasedontheirsimilaritytoneighboringobjects, havealsobeenutilizedinautomatedphenotypingtasks (FitriaWidiawatietal.2018).Furthermore,convol utionalneural networks (CNN) have emerged as powerful tools in automated phenotyping due to their ability tolearnfeaturesdirectlyfromrawdata, suchasimages (LeCunetal.2015). VariantsofCNNs, includingFastR -CNNandFasterR-CNN,havebeenexploredfordiverseobjectdetectionandclassification tasks including plant phenotyping (Ren et al. 2015, Lu and Cao, 2020, Liu et al. 2020). WhileFasterR- CNNoffersimprovedperformance,italsorequireshighercomputationwhichisabarrier.

Whenconsideringthechoiceofutilizingapre- trainedmodelforphenotypicanalysis, thevolumeofdatasetsavailableinanimmediatelyusablestatedon otnecessarilyresemblethedataformatsgeneratedinternallywithindifferentresearchprograms. Unlikes omepre-trainedmodels, whichexhibitarelatively highlevelofcomplexity andrequireasteeplearningcurve, alternativeapproachesandmodelscanofferamoreapproachableand modifiableframework.Thissimplicityreducesthebarriertounderstandingbutalsoprovidesimprovedint erpretabilitymitigatingthe′blackbox′effectassociatedwithmorecomplexdeeplearningmodels. Moreover, employing a simpler model allows for in-house modifications tailored to specificresearchneeds.

While advanced supervised models such as Faster R-CNN may offer sophisticatedfeatures, theadditionalcomputationalrequirementsdonotnecessarilyyieldsignificantim provements inresults comparedtosimpleralternatives (Rodeneetal. 2024).Manycloudbaseddataserviceschargeforthemanagementofsupervisionduringthelearningproc ess, addingtooverallcostandcomplexity.BasicCNNarchitecturesimplementedusingframeworkslikeKe rasprovide a more accessible, less computationally intensive, and easily applicable solution. The KerasCNN architecture used here was believed to be sufficient for the task while offering scalability andtransferabilityacrossRGBimagesthatdifferinnaturallightingconditions, resolutionandenvironmen t.Itwashypothesizedthatdeep- learningapproachesforhighthroughputtasselphenotypingareachievablewithoutrequiringhighly- complexcomputationalarchitecturewhichcanleadtoeasierincorporationandscalabilityacrossyearsan dlocations.

MATERIALS AND METHODS

2.1 Genomes to Fields Initiative (G2F) Maize Dataset

2.1.1 Field Design: Images from field experiments were collected in three fields in

College Station, TX, one field in Arlington, WI (WIH2) and one field in Madison, WI (WIH1) as part of the Genomes to Fields Initiative (G2F) in 2020 and 2021. Additional information on this experimentcanbe foundinLima etal. (2023). Inbrief, 220 hybridsand30 testlines weregrowninarandomizedcompleteblockdesign (RCBD) withtworeplicates,eachplotcomprisingonehy bridwithtworows (500plots perenvironment). InCollegeStation, TXandinArlington, WI thehybridswereallcrossedwithtesterparentPHZ51. InMadison, WIthehybridsgrownhadbeencrossedw ith tester parent PHP02. The specific data set in College Station, TX fields are as described in Adaketal. (2021a). InTexasthe500-plothybridtrialwasseparatelyevaluated underthreeseparatemanagement conditions for 1500 plots in total: TXH1 is defined as having optimal management andplantingdate, TXH2isdefinedasdrylandandreducedfertilizerwithoptimalplantingdate, andTXH3isd efinedasoptimalmanagementbutonemonthdelayedplantingdate.

2.1.2 Data Collection: UAS data acquisition of RGB images was made using a DJIPhantom4ProV2.0 (SZ DJITechnologyCo.Ltd.Shenzhen,China) equippedwitha1- inchCMOSRGBsensorwithamechanicalshutter.UsingtheDJIGSProapplication,flightmissionswerepla nnedwiththefollowingparameters: 25melevation (aboveground),90% forwardoverlap,80% side overlap, flight speed of 1.2 m/s, and a shutter interval of 2.0 s. Raw images were sortedintoseparate173foldersnamedaccordingtoeachflightdateinpreparationforconstructionofort homosaics within Agisoft Metashape (Agisoft LLC). Lower flight heights allow for higher groundsampling175distance/resolution.

2.1.3DataProcessing: Asummaryoforthomosaickingandgeoreferencingprocedure sisdescribedas: a) folderscontainingRGBimageswereloadedintoMetashape;b) photoalignmentwasco nductedusing60,000keypointsandOtiepointswithreferencedpreselection; c) an initial bundle adjustment was performed to optimize the f, cx/cy, k1, k2, k3, p1, andp2distortionparametersofthelens;
d) iterativemodelerrorreductionprocedures (termed” gradualselection “inMetashape) wereperformed toremoveerroneouspointsfromthesparsecloud;e) groundcontrolpoints (GCPs) wereimportedandman ualalignmentoftheirlocationswithinatleastsixRGBimageswasperformed followed by selecting the “update” option within Metashape to integrate the GCPs intothemodel;f) cameraalignmentwasperformedagainwithallavailabledistortionparameters;g) the dense point cloud was processed with “moderate” depth filtering at “medium” quality with allothersettingsleftasdefault;h) colorcalibrationwasperformedusingthesparsecloudas the sourceandthe “calibratewhitebalance” optionwasalsochecked;i) thedigitalelevationmap (DEM) was calculatedusingthedensecloudas thedatasourcewithall othersettingsleftasdefault; j) the orthomosaic was produced using the DEM as the surface with all other settings leftasdefault.AllgeoreferencedfinalproductswereexportedinWorldGeodeticSystem1984 (WGS84) d atum (specifically,UTMZone14NforTexasand16NforWisconsin) coordinates.

Orthomosaics and DEMs were exported from Metashape and saved with the.tif extension, whilethedensepointcloudwassavedwithboth.lasand.lazfileextensions.

Intotal, thedatasetconsistedof15,000.tifimagescollectedfromthirtyflightsconducte dacrossmultipleyearsandenvironments.Onlyflightsduringtheinitiationoftasselingwere selected. In 2020, four flights of fields TXH1 & TXH2 and three flights of TXH3 were used (FIG. 22A). In2021,fiveflights ofTXH1&

TXH2, threeflightsofTXH3,threeflightsofWIH1, andthreeflightsofWIH2wereconducted.Eachflighttoo k acompositeofRGBimagescontainingall500plotsatdifferingquality.Ofthe15,000totalimages, thedatas etbecame13,491imagesafterremoving images that were greater than 50% soil or had no recorded DTA (Table 5). 215 meaninglessthanfiftypercentfloweredor′1′meaningmorethanfiftypercentflowered, categories asestimatedvisually. Thedateof50% anthesis, as daystoanthesis (DTA) and50% silkingasdaystosilk (DTS), wererecordedfromvisualobservationsmanuallywalkingthefiel deverytwotothreedaysonceanytasselswerefirstobserved. Thetasselinitiationdatewasunfortunately notrecordedinthesemanualscoringsbutwereexpectedtobeafewdays beforeDTAandhighlycorrelated.Days to tasseling (DTT) wascalculatedas thedaysafter planting (DAP) corresponding to the first flight date where tassels are visually present in more than 50% of maizeplants.

2.1.5 Summary Statistics: The correlation between DTT and DTA was

determinedinR.ArandomeffectsmodelANOVAwasperformedusingthelme4packagetodeterminerep eatabilitymeasurementsofDTAandDTTforallenvironmentsexcludingMadisonwhereadifferenttester was usedaccording toEq. 1as wellasby treatmentaccordingtoEq.2inTexaswherethereweremultipleyearsandEq.3inWisconsinwheretherew asonlyoneyear.

$${Y}_{jklmn} = μ + {Pedigree}_{j} + {Env}_{k} + {Range}_{l ( k )} + {Row}_{m ( k )} + {Rep}_{n ( k )} {+}_{jklmn} \\ Equation 1 ; \qquad Eq . 1$$

Y is a vector of length 230 (the sum of the number of hybrids) denoting each traitvalue (DTTorDTA) inDAPforeachhybridinone ofeightenvironments.Heredenotes thegrandmean; Pedigree denotes the overall effect of the jth maize hybrid, Env denotes the overall effect ofthekth treatmentenvironment;Rangedenotestheeffectofthelth rangenestedinthekthenvironment; Row denotes theeffectof the mth row nestedin the kth environment; Rep denotesthenth replicationnestedinthekth environment;and [denotestheerrortermaddressingnonexplainedvariability.

$${Y}_{jklmn} = μ + {Pedigree}_{j} + {Year}_{k} + {Range}_{l} + {Row}_{m} + {Rep}_{n} {+}_{jklmn} \\ Equation 2 ; \qquad Eq . 2$$

Equation 2 differs from Equation 1 such that Year is included as an effect whileRange,Row, andReparenolongernestedeffects asanalysisis being performedonaperenvironmentbasis.

$${Y}_{jklmn} = μ + {Pedigree}_{j} + {Range}_{l} + {Row}_{m} + {Rep}_{n} {+}_{jlmn} Equation 3 ; \qquad Eq . 3$$

Equation 3 differs from Equation 1 such that each environment (year by location) is analyzedseparatelyandthereforeRange,Row, andReparenolongernestedeffects.

Repeatability was calculated according to Eq.4. Here, & and 2indicate the variance components of the effects of genotype and the error term, respectively, and n is thenumberofreplicates:

$$Repeatability = \frac{{:}_{G}^{2}}{{:}_{G}^{2} + \frac{{>}_{?}^{2}}{n}} ; Equation 4 , \qquad Eq . 4$$

11

TensorFlow, seaborn, matplotlib.pyplot, and imageio.v3. Image files were namedwithadatecodeformat “YYYYMMDD- “whichallowedforindividualyearsandlocationstobesubdivided.

Givenlargedifferencesinflightdatesbetweenenvironments,specificregionscouldbe selectedbyyear (e.g.2020or2021) orbythecombinationofyearandmonthtogether (e.g.202105or2021 07).Imageswereread,resizedto500x500pixels, andcheckedtoensurethey had the dimensions 500 x 500×3 representing only the RGB bands and removing any alphachannels beforebeingstoredinanarray.This arrayhadall pixelvaluesof′NA′setto′ 0′ (anartifactofremovingsoilwithfieldlmageR) beforenormalizingallpixelvaluesfrom.tifformat images by dividing by 255 (the maximum pixel brightness value) such that all pixel values fell withinarangeof [0,1].

Eachmodelwasrepeatedtentimesincludingtherandomlyiteratedtrain/testsplit for image files from Texas in 2020 which was 80% training 20% test. Texas 2020 was the onlysetusedfortraining. Trainingofthemodeloccurredoverfiftyepochs, withsubsequentclassifications madeonpreviouslyunseenimagesfromTexas2020,Texas2021, andWisconsin2021 datasets. Performance of the DL approach was analyzed according to the following equations:

Precision (Eq.5) measurestheaccuracyofpositivepredictionsmadebythemodel. It was calculated as the ratio of true positive predictions to the total number of positivepredictionsmadebythemodel, includingbothcorrectandincorrectpositivepredictions.

$$Precision = \frac{True Positive}{True Positive + False Positive} ; Equation 5 , \qquad Eq . 5$$

Recall (Eq. 6) measures the completeness of positive predictions made by themodel.Itwascalculated astheratiooftruepositivepredictions tothetotalnumberofactualpositiveinstancesinthedataset.Mathematically, recallisrepresentedas . . .

$$Recall = \frac{True Positive}{True Positive + False Negative} ; Equation 6 , \qquad Eq . 6$$

TheF1-score (Eq.7) istheharmonicmeanofprecisionandrecall.Itprovidesabalance between precision and recall, giving equal weight to both measures. F1-score is calculatedusingthefollowingformula:

$$F 1 - score = 2 \times ( \frac{Precision \times Recall}{Precision + Recall} ) ; Equation 7 , \qquad Eq . 7$$

Accuracy (Eq. 8) measures the overall correctness of the model's predictions.

Itwascalculatedas the ratioofcorrectpredictions (bothtrue positivesandtrue negatives) to thetotalnumberofpredictions.Mathematically,accuracyisrepresentedas:

$$Accuracy = \frac{True Positive + True Negative}{Total} ; Equation 8 , \qquad Eq . 8$$

Inordertoproperlysavemetrics, emptylistsforevaluation, metrics, confusionmatric es,predictedlabels, andF1-scoreswereinitiated.Floweringstatusvalueswereone-hotencoded before the CNN model was executed. The CNN model architecture was constructed afterperforming hyperparameter optimization (FIG. 23) and is as described in Table 6.

HyperparameteroptimizationwasperformedusingtheOptunapackageinPython (Akibaetal.2019) and determinedthatthemostimportanthyperparameterwashavingfiveconv2Dlayers. Thegoalof hyperparameter optimization is to isolate the specific combination of adjustable variables that willproduce the best accuracy while minimizing loss. Loss shows the difference between the predictedvalueandthegroundtruthvalue, andthedifferencebetweenlossandvalidationlossisausefulm etric for gauging over- or underfitting. The line ‘tt.keras.backend.clear_session ( ) was included attheendoftheloopinordertooptimizememoryusage.

ConvlayersareallConv2D.: Evaluationofmodelperformanceincludedthegeneration andstorageofconfusionmatricesandsaliencymapstovisualizeclassificationresults.Saliency maps were created by copying the model to extract and visualize specific layers for specificimages, allowing theexportoflayersthatshowactivationforeithermaizeshowing tassels ornot.Metricswere301concatenatedintostructuredlistsandexportedtoCSVfilesforcomprehensiveana lysis and 302 documentation. F1-scores were then compared against previously published deeplearning303studies. Labelslistsweregenerated andsortedtoshowwhichflightdayswerethefirsttorecord304tasseling. individually (Table 3b), and all environments individually.

RESULTS

3.1 Summary Statistics

3.1.1 Statistical Analysis of DTT and DTA: Correlation (r2) between DTT and

DTAbasedonthedatainLimaetal. (2023) wasfoundtobe0.71 (FIG. 24).ANOVAwas performedontrials all using the same genotypes (TXH1, TXH2, TXH3, and WIH2 datasets) for DTT and DTA (Table7a), aswellas forWIH1tasseling (DTT) anddaystoanthesis (DTA).322dataset (FIG. 25A- 25B).Itwasalsohighlyaccurate (0.911and.981) onindependenttestdatasets 323inTexas2021andArlington, Wisconsin (FIG. 25C). Precision, Recall, and F1-scores for 324 corresponding datasets arepresentedinFIG.25D.Saliencymapsweregeneratedshowingtheactivationofdifferentconvolutional ordenselayersformaizeplantswithandwithouttassels (FIGS. 26A- 26B). Layerswithtasselspresentshowmoreactivationthanthesamelayerwithouttassels.

3.2.2Madison, WITasselDetection: In2021aspartofG2Fbutwerenotpreviously used for training in this study. These used the same inbred lines but a different tester (PHP02) tocreatethehybrids.Imageswerescoredmanuallyandthenlabeledusingtenreplicatio ns of randomly partitioned train/test splits from the TX 2020 trained model. The model was very accurate (0.988) at determining flowering status for the unrelated hybrids in Madison,

Wi (FIGS. 27A-27B).

### DISCUSSION

TheCNNmodelusedhere, whentrainedonlargedatasets,demonstratedeffectiveness inautomatingphenotypingtasksincludingthedetectionoftasselingplotsinmaizefieldswithoutnecessita tingmorecomplexcomputationaltools (Table8).Byautomatingtheidentificationoffloweringmaizeusin gUAS-basedimagery, theobjectivewastoreplacethetime- consumingandsubjectiveprocessofmanualrecordingdaystoanthesisor tasselidentificationfrom images with a more efficient and objective method. The limited image resolution of most UASdata sets, including this one, is insufficient to observe the presence of pollen released from anthers (DTA).However, tasselsarereasonableproxiesandcouldbeobservedintheseimages.

Beyondtreatingtheimagecollectiondatesasindependent, thedateoftasselappearan cecouldbeextractedfromthesedeeplearningmethodsbyarrangingpredictedclassesby dateand findingthe firstoccurrence of alabel indicating the detection of tassels, however thisisonly practicaliftheflightsarespacedcloselyenough. Tasselemergence isarelativelyrapidprocess, thereforeinordertodeterminetasseldatebyUASimageryflightsshouldoccur dailywithintheflowering windowofall testedvarieties.For example, iftasselinitiationoccurredat73daysafter planting butflightsonlyoccurredondays72and78, the tasselemergence wouldn′tbecaptured until day 78. Conversely, if flights were made multiple times a day, the precision of tasselemergencetotimewithinadaymightbeabletobedetected.Thiswould haverelevancetoquantitativegeneticstudiestodissectloci withsmallereffects thanasingledayas itappears themajority ofsegregating floweringloci demonstrateinmaize (Buckleretal. 2009).Alternatively, togettimingoftasselappearancewithsparseUAS data,futuredeeplearningmodelscouldgobeyondclassificationtodevelopquantitativemodelsforestim atesoftasseling;eitherasthepercentageofplantsper plottasselingorthepercentageofthetasselsemergedwithinplantsintheplot.However, theseapproache swouldrequiremanualestimatesonindividualplantsormanual quantitative estimates of how much the tassel has emerged to sufficiently train the model.GiventhehighrepeatabilityobservedforDTT (andDTA) demonstratedhere, themanualandpredi ctedclassificationofDTTwasahighqualitymeasureanditwouldmakesensetoinvestinquantitativeestim ation.

ComparingresourceinvestmentsbetweenDTTandDTAiscomplicated,dependingont echnologyavailableandused. Theroughestimateshere (Table8) showthatDTTtook moreeffort, andhighereducation levels thanDTA, which can easily bemeasured by studentworkerswithlimitededucationandtraining.However,itisworthnotingthatUAScollectionandan alysis technologies will continue to improve in speed and accuracy for DTT, while DTA cannot befurtherscaledorimproved.Manyotherphenotypesandpredictionscansimultaneouslybemadeusing the same UAS data (Gano et al. 2024), such as plant height (Anderson et al. 2019, Pugh et al.2018, Tirado et al. 2020), yield predictions (Kumar et al. 2023, Sunoj et al. 2021, Barzin et al. 2020),disease (Wuetal.

2019,Chivasaetal.2021, DeSalvioetal.2022), andfurtherenhancetheunderstandingofthephenome (M urrayetal.2023).Nevertheless, arecentsurveyidentifiedthe” high cost of instruments/devices or software” and the “Lack of knowledge or trained personnel toanalyzedata” asimportantbarrierstoUASadoption (Lachowiecetal.2024). aAccuracydidnotimproveafter20epochs.

Thedecisionheretouseasimple, modifiablemodelasopposedtopre- trainedalternativesoronewithhighercomputationalrequirementsalignswiththegoalofmakingdeeplea rning-based phenotypic analysis more accessible and cost-effective without sacrificing accuracy. The affordability and versatility of RGB cameras further support this experience, enabling programswithlimitedresourcestostillbenefitfromincorporatingmorephenotypicdataintoresearchen deavors. Unlikehyperspectralcameras, whicharemoreexpensiveandrequirespecializedequipment,RG Bcamerasofferanopportunitytoreadilyincorporatehigherimageresolutionandmorephenotypicdatas uitablefordeeplearningtechniques.ProgramsusingRGBcanthereforecapitalizeonthepotentialforlarge rdatasets,evenwithconstrainedresources, byimplementingthese methods more quickly.

Multispectral camera data, incorporating a few additional bands overRGBwithcomparablespeedandresolution, couldfurtherimproveresultsandareworthinvestigating for DTT. However, this canrequire the useofmore expensivetechnology,potentiallyprohibitiveforsmaller-scaleprogramsandproducers. WhilecodingthisapproachinPython,differentsituationswereencounteredwherethe architectureoftheCNNwouldleadtoerroneouspredictions.Activationmapscanbeinspectedforproperc onvolutionallayeractivationatthetraitofinterest aswellasconfusionmatrices with predicted labels. In situations where the flowering status of unscored images is being classified, manualassessmentcanbeusedonanyorall images thatdonotreceiveaconsistentpredicted label. In doing this it was observed that orthomosaic stitching created artifacts in certainsegmentsofthefield,generallyduetowindyconditionsorpooroverlapbetweenimages. Furthermore, poorimageresolutionledtolessconsistentclassifications. Thedevelopmentofauser- friendlymethodologysuitablefordeploymentamongadiverserangeofusersorproducers, regardless of their computational experience, accessibility, or motivation to incorporate Al, has thepotential tonotonlyenhanceaccuracybutalsosavetimeandimproveresultsinthelong run.

Vegetative indices are more informative than raw bands in terms of signal-to-noise ratio and usefulfor detecting phenotypesinbandsoflightthatarenotpresentintheRGB spectrum (Danileviczetal.2021). Incorporatingvegetativeindicesandmultispectralimageryshouldalsobeconsider edinfuturestudies.

Thecorrelationanalysisrevealedastrongrelationshipbetweendaystoanthesis (DTA) a nddaystotasseling (DTT),withhighR2 (0.71) andgoodcorrespondenceacrosstheentiredataset.Repeatab ilitybetweentraitsinthesamedatasetisamoreobjectivemetricsinceitevaluates consistency of an observation that is not due to chance, and is not dependent on anothermanualmeasure, whichmayhaveitsownerror, likeR2.Whilethecombinedanalysisdemonstrate dsimilarmoderaterepeatabilityvaluesforDTA (0.651) andDTT (0.541), therepeatability in individual environments varied substantially (0.365-0.885) for DTA and (0.141- 0.790) forDTT. Therearetwoprimarypotentialcausesofreducingrepeatability.Onebeingmeasuremente rrors fromhumans (DTA) ortheCNNmodel (DTT), theotherisareducedwindowofgeneticvariation. DTT hastwo additional potentialcauses ofreducedrepeatability asmeasuredhere, thetemporalgranularityoftheflightsandtheuseofclassification.CombinedDTTcouldn otberevisitedorprojectedforwardorbackwardslikemanuallyestimatedDTAcan,compressingthevariati on. For example, in the combined analysis there is a range of 58 to 81 days for DTT, but 52 to82daysforDTA.

The phenotyping of DTA in Wisconsin was better suchthat higher repeatabilitiesin WI were observed despite a smaller range of flowering time (20 days) compared to TX (30 days). The measurement error was especially notable in TXH3 DTT which had both compressed variationinflowering (10 days) duetoheat, andlowerresolutionimages (repeatability=0.141).

WhenexaminingtherepeatabilityofDTA, lowvalueswereobtainedinTXenvironments (0.141-0.549) comparedwithWlenvironments (0.365- 0.885), suggestingsubstantialmeasurementerrorinobservationsofthistrait.Thisdiscrepancymayindica tehighamountsoferrorrather thanalackofastronggeneticcomponenttothesetraits.Repeatabilityestimatesfor both DTA (0.885) and DTT (0.790) werehighest inWIH2 and therepeatability of DTTinWIH1 (0.572) exceeded that of DTA (0.365). Flights in these environments were more frequent duringthefloweringwindowandimagesweretakenatahigherresolution. Theoveralllowrepeatabilityest imatesforDTAmeasurementscouldbecausedbyhumanerrorthroughdiscrepancyinvisualdetectionofs mallpollengrainsorsubjectivedetermination, whileerrorinDTTwaslikelyfromorthomosaicstitching,lo wimageresolution, andlackofflightsconcurrentwithtasselinitiation.

TherelativelyhighpercentageofvarianceattributedtotheYearcomponentforDTTinTexas islikelyduetothelargevariationingroundsamplingdistance (6.8mmperpixelin2020versus10.9mmperpi xelin2021).Giventhesefindings, futurestudies shouldprioritizetheacquisitionofhigh- resolutionimageswithneardailyflightswithintheanticipatedfloweringwindowofeachtreatment to enhance data accuracy and repeatability. Furthermore, if high enough resolution wasobtained, it is conceivable that anthers could be detected on the tassel, unifying the CNN approachwithconventionalDTAmeasures.

Thisstudyhighlightsthepotentialfortemporalphenotypingbyextractingadditionalph enotypictraitsthroughoutthegrowingseason.Thiscouldhappeninnear- realtime, onlylimitedbydataprocessing, orashere, retrospectively.Thiscanprovidenewinformationand phenotypesonspecificplotsandgenotypes.Notablythesenewmeasurescanhelpbreedersandplantbiol ogistsgainvaluableinsightsintothedynamicsofplantdevelopmentandpotentiallystressresponseswhe nthesephenotypesareobservableinRGBimagery.Additionally, thisapproach, once validated by other researchers, may offer opportunities for timely intervention andmanagementdecisionsforproducers;themostobviousexamplebeingdetasselingdecisionsforhybri dseedproduction.

Ascomparedtoexistingmethodsofextractingfloweringtime,establishingaground truth in a field by counting tassels requires manual counting, which is both labor- intensiveandexpensive.Thisstudydemonstratedthataqualitativevisualtraitphenotypedinoneenviro nmentcaneasilybeextendedtoanotherusingdeeplearningtoreducefuturecostsand labor. Infact, theimprovedestimationofWisconsin, basedonTexas-traineddatademonstratedthat image and data quality can improve estimation accuracy beyond the original training dataset.PhenotypingusingUASimagerymayincurinitialexpensesassociatedwithequipmentpurchases butwillallowforhigh- throughputphenotypingatareducedhourlycost.Furthermore, historicalimageryallowsretrospectived ataanalysisfornewphenotypesastheyaredeveloped. Thereisinherentvalueto a high volume of data, regardless of quality beyonda certain baseline (LaneandMurray2021).Eventually,asmorebreedingprogramsacquireandroutinelycollectUAS technology,easy-to-useprotocolsforprocessingthevastvolumesofdataproducedwillbeessential in incorporating phenomic data into multi-omic analyses (Chen et al. 2022). It should alsobepossibletoapplythismethodologytounoccupiedgroundsystemsormanuallyacquiredimagerypr ovidedtheinputimagesappearsimilar tothedataset.Automationoftasseldetectionnotonlyenhancesthescalabilityandspeedofphenotypingb utalsoreduceshumanerrorandvariability.Byestablishingavisuallyscoreddatasetformodeltrainingandt henvalidatingthistrainedmodelusingdatafromunknowngenotypesandenvironments, thechallengesa ssociatedwithaccuratelydetectingthecomplexfloweringphenotypeswereaddressed.

DevelopingConnectionstothePhenome: Thestudyfurthershowedthattherearenove Iheritablebiomarkersdetectablebydeeplearning.Again, thenovelbiomarkerscanbeadded to the set ofall biomarkers (e.g., knownexisting biomarkers) to saturate the phenome withbiomarkers that can be predictive of important phenotypes using composite scores from phenomicbiomarkers.

### Tables

TABLE 1
Distribution of senescence scores belonging to each category across
experiment 1 (E1) and E2 revealed that many plants displayed either
stay-green or a resurgence in vigor after an initial period of senescence.
Data augmentation included images undergoing a horizontal flip and
three clockwise rotations of 90°, 180°, and 270°.

| Visual | | | | | | |
|---|---|---|---|---|---|---|
| Senescence | | | | | | |
| Rating | 0 | 1 | 2 | 3 | 4 | 5 |
| E1 | 1,129 | 939 | 448 | 196 | 109 | 469 |
| E2 | 1,303 | 1,096 | 474 | 127 | 59 | 273 |
| Combined | 2,432 | 2,035 | 922 | 323 | 168 | 742 |
| E1 Augmented | 5,645 | 4,695 | 2,240 | 980 | 545 | 2,345 |
| E2 Augmented | 6,515 | 5,480 | 2,370 | 635 | 295 | 1,365 |
| Combined | 12,160 | 10,175 | 4,610 | 1,615 | 840 | 3,710 |
| Augmented | | | | | | |

MCCRef.No.: 11164-020US1

TABLE 2
The parameter space through which Optuna searched
duringhyperparameteroptimizationispresented.

| Hyperparameter | Type | Range/Choices | Notes |
|---|---|---|---|
| Dropout rate | Float | 0.0 to 0.5 | Uniformly distributed |
| Learning rate | Float | 1 × 10^{-s} to | Logarithmically scaled |
| | | 1 × 10⁻² | |
| Regularization | Float | 1 × 10⁻⁴ and | Logarithmically scaled |
| | | 1 × 10⁻² | |
| Activation | Categorical | ‘relu’, | AppliestoConv2Dlayersforfirstsetofmodels; |
| function | | ‘tanh’, | applies to Conv2D layers and |
| | | ‘linear’ | firstoftwodenselayersforsecondsetofmodels |
| Dense neurons | Categorical | 128, 256, | N/A |
| | | 512, 1024 | |
| First kernel size | Categorical | 2, 3, 4, | Size of kernel in first Conv2D layer |
| | | 5, 6, 7 | |
| Initial filters | Categorical | 16, 32 | Number of filters in first Conv2D layer |
| NumberofConv2D | Integer | 1 to 5 | N/A |
| layers | | | |

TABLE 3
CNNmodelnames, regressiontargets, andnotesexplainingthedifference
between the first set of models (M1-M3) and the second
set (M4-M6). The regressiontargets are FPC1scoresoftemporalsenescence
derived from pooled data from experiments E1andE2for:
visualsenescenceratings(VSRs), RCC, orTNDGRvegetationindexvalues.

| | Regression | |
|---|---|---|
| Model Name | Target Value | Activation Function Configuration |
| M1 | VSR FPC1 | Conv2D layers: searchable by Optuna; |
| | | dense layer 1: ReLU |
| M2 | RCC FPC1 | Conv2D layers: searchable by Optuna; |
| | | dense layer 1: ReLU |
| M3 | TNDGR FPC1 | Conv2D layers: searchable by Optuna; |
| | | dense layer 1: ReLU |
| M4 | VSR FPC1 | Conv2D layers: searchable by Optuna; |
| | | dense layer 1: searchable byOptuna |
| M5 | RCC FPC1 | Conv2D layers: Searchable by Optuna; |
| | | dense layer 1: searchable byOptuna |
| M6 | TNDGR FPC1 | Conv2D layers: Searchable by Optuna; |
| | | dense layer 1: searchable byOptuna |

TABLE 4
Model performance metrics for M1-6 calculated using actual and predicted
values from CNN regression. Data are grouped according to regression
target variables due to the differences in scale associated with each
target that affect the interpretation of root mean squared error (RMSE),
mean absolute error (MAE), mean squared error (MSE), and MAPE.

| Regression Target | Model | R² | R | RMSE | MAE | MSE | MAPE (%) |
|---|---|---|---|---|---|---|---|
| VSR | M1 | 0.857 | 0.926 | 3.12 | 2.38 | 9.72 | 1.12 |
| | M4 | 0.886 | 0.941 | 2.76 | 2.09 | 7.61 | 1.19 |
| RCC | M2 | 0.619 | 0.787 | 0.0396 | 0.0298 | 0.00157 | 18.9 |
| | M5 | 0.435 | 0.659 | 0.0485 | 0.0367 | 0.00235 | 19.6 |
| TNDGR | M3 | 0.743 | 0.862 | 0.0832 | 0.0631 | 0.00692 | 5.94 |
| | M6 | 0.745 | 0.863 | 0.0787 | 0.0575 | 0.00620 | 5.44 |

TABLE 5
Image distribution and resolution.

| Treatment(Field) | Number | Ground Sampling Distance |
|---|---|---|
| TXH1 2020 | 2000 | 6.8 mm/pixel |
| TXH2 2020 | 1491 | |
| TXH3 2020 | 1000 | |
| TXH1 2021 | 2500 | 10.9 mm/pixel |
| TXH2 2021 | 2500 | |
| TXH3 2021 | 1000 | |
| WIH1 2021 | 1500 | |
| WIH2 2021 | 1500 | |

TABLE 6
CNN Architecture.

| Layer name | Output size | Operations |
|---|---|---|
| conv1 ^{a} | 498 × 498 | [3 × 3, 32, relu] |
| pool1 | 249 × 249 | 2 × 2 max pool, stride 2 |
| conv2 | 247 × 247 | [3 × 3, 64, relu] |
| pool2 | 123 × 123 | 2 × 2 max pool, stride 2 |
| conv3 | 121 × 121 | [3 × 3, 128, relu] |
| pool3 | 60 × 60 | 2 × 2 max pool, stride 2 |
| conv4 | 58 × 58 | [3 × 3, 256, relu] |
| pool4 | 29 × 29 | 2 × 2 max pool, stride 2 |
| conv5 | 27 × 27 | [3 × 3, 512, relu] |
| pool5 | 13 × 13 | 2 × 2 max pool, stride 2 |
| flatten1 | 338 | |
| dense1 | 256 | [256, relu] |
| dense2 | 2 | [2, sigmoid] |

TABLE 7a
ANOVA results for DTA and DTT of the entire dataset excluding Madison.

| | DTA | DTA | DTA | DTT | DTT | DTT |
|---|---|---|---|---|---|---|
| | vcov | sdcor | Percent | vcov | sdcor | Percent |
| Pedigree | .632 | .957 | .028 | .413 | .643 | .012 |
| Treatment:Row | <.001 | <.001 | <.001 | <.001 | <.001 | <.001 |
| Treatment:Range | .092 | .302 | .004 | .251 | .501 | .008 |
| Treatment:Rep | .650 | .806 | .028 | .281 | .531 | .008 |
| Treatment | 16.24 | 4.03 | .705 | 26.68 | 5.16 | .802 |
| Residual | 5.42 | 2.33 | .235 | 5.62 | 2.37 | .169 |
| | RMSE | RSquared | Repeatability | RMSE | RSquared | Repeatability |
| | 2.247 | .764 | .651 | 2.299 | .831 | .541 |

TABLE 7b
ANOVA results for DTA and DTT for Madison, WI (WIH1).

| | DTA | DTA | DTA | DTT | DTT | DTT |
|---|---|---|---|---|---|---|
| | vcov | sdcor | Percent | vcov | sdcor | Percent |
| Pedigree | .441 | .664 | .191 | .252 | .502 | .370 |
| Row | .276 | .525 | .119 | .053 | .231 | .078 |
| Range | <.001 | <.001 | <.001 | <.001 | <.001 | <.001 |
| Rep | .056 | .237 | .024 | <.001 | <.001 | <.001 |
| Residual | 1.53 | 1.24 | .665 | .377 | .614 | .552 |
| | RMSE | RSquared | Repeatability | RMSE | RSquared | Repeatability |
| | 1.08 | .335 | .365 | .480 | .448 | .572 |

TABLE 8
F1 scores of varying DL approaches that identify
maize tassels. Higher (0-1)scoresarebetter.

| Study | DL approach/architecture | F1-scores |
|---|---|---|
| Zan, 2020 | RandomForest & VGG16 | .94 |
| Karami, 2021 | CenterNet, TSD, DetectoRS | .932, .899, .903 |
| Alzadjali, 2021 | Faster R-CNN (ResNet50) | .979 |
| Alzadjali, 2021 | TD-CNN (Inception v3) | .959 |
| Shepard, 2024 | CNN (Keras) | .96, .935, .99 |

TABLE 9
Time estimates for DTT by human and computer, DTA by human.

| | | DTT | DTA manual |
|---|---|---|---|
| Task | DTT human time effort | computationaltime | estimates |
| Manual scoring | 30 minutesperflight × 4flightstocapture | (90 s per epoch at | 0.25 minutes perplot × |
| | DTTperfield = 2 hours | 20epochs^{a})30 minutes × | 5 days per field = |
| | | 10 replications = 5 | 313 person hours |
| | | hours | |
| UAS flights | .5 hours per flight × 4 | NA | NA |
| | flights to captureDTT per | | |
| | field = 2 hours | | |
| Orthomosaicing | .25 hours × 4 flights = 7.5 hours | 1 hour per flight × 4 | NA |
| | | flights = 4 hours | |
| Shape file creation | .25 hours × 4 flights = 1 hour | NA | NA |
| Plot extraction | <.5 hour | NA | NA |
| Deep learning model | 24 hours of programming, | 12 hours hyperparameter tuning + | NA |
| trainingandprediction | troubleshooting | 12 hoursfortrainingandpredictions = | |
| | | 24 hours | |
| Analysis | 2 hours | NA | 2 hours |
| Total | 42 hours | 33 hours | 315 hours |

### Example 4: Animal Phenotyping

Commonissuesinanalyzingananimalorgroupofanimalsinclude: determiningthebree dofanimal (“admixturegenetics”), determiningtherelationshipsbetweenanimals (“pedigreegenetics”), determiningtheageofanimals (biologicalage), howwelltheanimalsaretakencareof (e.g., environmental and/orowner/caretakereffects), dispositionsoftheanimals (e.g., geneticandenvironmentalinteraction s), and/orhowtocareforanimals (e.g., personalizedmedicine).Questionsofbreedandrelationshipcanbe determinedbygenetictesting, butotheranalyses may not be solved by genetic testing alone. The methods of the present disclosure can beapplied to animals or groups of animals to perform these analyses and predict these and any othertraits. It should be understood that various combinations of these analyses apply to many differenttypes of animals, including humans, although the present example describes them in the context ofdogs.

Implementationsofthepresentdisclosurecanbeconfiguredforanalyzingandmaking predictionsabouttraitsofanimals (e.g., dogs).For example,manydomesticanimalsarecommonlyphoto graphedandvideo- recorded, generatinglargeamountsofdataaboutthephenotypesoftheanimals.Thisdatacanbeseparat edtemporally (e.g., overdays, months, oryears) andcanincludedifferentviewsofthesameanimal (e.g., fromdifferentangles, underdifferentlightingconditions).Additionally,animalscanbephotograph edand/orrecordedovertime for medical reasons (e.g., at a veterinarian). For example, photos of teeth, hair, nails, etc. canbeacquired.

The images over time can be used to extract temporal phenomic biomarkers thatcan be used to evaluate environmental and gene/environmental interactions. Additionally, traits ofinterestcanbeacquiredfromgeneticand/orveterinarytesting.

As an example, the example implementation for canine phenotyping can be usedtoimproveonexisting “brute- force” methodsofAlfordetectingdogbreedsand/orotherdogtraits.Existingmethods maynotconsiderthephenotypeoftheanimalovertimebyconnectingmultiplephotostogether, may not beabletohandlenewcases, and/ormayrequireseparateAlmodelsforeachsubjectofinterest (e.g., breed, relationships, aging, temperament, nutrition, medicine,etc.).

An example implementation of the present disclosure includes a model trained todeveloppredictivebiomarkers for dogs. The training sets canincludeimages of thesamedog over time. Theexampleimplementationcanextractbiomarkersusingknownfeaturesand/orAlapproaches (e.g., as model variables). The training set can optionally further include phenotypes ofinterestofthesamedog, forexamplebehavior, health,responsetodiet/medicine,etc. Thetrainingsetca noptionallyfurtherincludegeneticdata (e.g., breedandrelationshiptestingofmixed breeds). The genetic data can be used to further determine environmental effects, effects ofthegenetic/environmentalinteractions, and/orbreed-specificeffects.

Themodelcanbedevelopedbymeasuringanynumberofbiomarkersfromtheimages.

Optionally, alargenumberofbiomarkersareused, forexample thebiomarkerscanincludeall thebiomarkersthatasystemcanpracticallyextractfromanimage.Itshouldalsobeunderstoodthattheme thodsforextractingbiomarkerscanincludeanyofthemethodsdescribedin the present disclosure to measure/discover biomarkers. Phenotypes of interest can be estimatedfromcompositescoresofbiomarkers,forexamplebymachinelearning.

Theexampleimplementationcanfurtherincludeapplyingthemodel.For example, a user can input images and/or video of a dog to get an interpretable predictions (e.g. ofphenotypesofinterest) basedonbiomarkers, andthepredictionscanincludeuncertaintyestimatesba sedonthebiomarkers.

### REFERENCES

Although the subject matter has been described in language specific to structuralfeatures and/ormethodologicalacts, itis tobeunderstoodthatthesubjectmatterdefinedintheappended claims is not necessarily limited to the specific features or acts described above. Rather, the specific features and acts described above are disclosed as example forms of implementing theclaims.A,S.,&Sangeetha,J. (2021).SmartIrrigationandprecisionfarmingofpaddyfieldusingunmann edgroundvehicleandinternetofthingssystem. InternationalJournalofAdvancedComputerScienceand Applications,12 (12).

Aasen, H., Kirchgessner, N., Walter, A., & Liebisch, F. (2020). PhenoCams forfield phenotyping: Using very high temporal resolution digital repeated photography to investigateinteractionsofgrowth,phenology, andharvesttraits.FrontiersinPlantScience,11.

ADAK, A., MURRAY, S. C. & WASHBURN, J. D. 2024. Deciphering temporal growth patterns in maize: integrative modeling of phenotype dynamics and underlying genomicvariations.NewPhytologist,242,121-136.

Adak, A., Murray, S. C., Anderson, S. L., Popescu, S. C., Malambo, L., Romay, M. C., & de Leon, N. (2021). Unoccupied aerial systems discovered overlooked loci capturing thevariationofentiregrowingperiodinmaize. ThePlantGenome, 14 (2).

Adak,A.,Murray,S.C.,Božinović,S.,Lindsey,R.,Nakasagga,S.,Chatterjee,S., Anderson, S. L., & Wilde, S. (2021). Temporal vegetation indices and plant height from remotelysensedimagerycanpredictgrainyieldandfloweringtimebreedingvalueinmaizeviamachinele arningregression.RemoteSensing, 13 (11),2141.

Adak, A., Murray, S.C. and Anderson, S.L., “Temporal phenomic predictionsfromunoccupiedaerialsystemscanoutperformgenomicpredictions, “BioRxiv,G3accepte d (2021).

AKIBA, T., SANO, S., YANASE, T., OHTA, T. & KOYAMA, M. Optuna: A next- generation hyperparameteroptimization framework. 20192019. 2623-2631.

Akiba, T., Sano, S., Yanase, T., Ohta, T., & Koyama, M. (2019). Optuna.

Proceedings of the 25th ACM SIGKDD International Conference on Knowledge Discovery & DataMining.

Allen, G. E., “Mendel and modern genetics: the legacy for today,” Endeavour,27 (2),63-68 (2003).

Alvergne,A.,Huchard,E.,Caillaud,D.,Charpentier,M.J.,Setchell,J.M.,Ruppli, C., Féjan, D., Martinez, L., Cowlishaw, G. and Raymond, M., “Human ability to recognize kinvisuallywithinprimates, “InternationalJournalofPrimatology,30 (1),199-210 (2009).

Alzadjali, A., Alali, M. H., Veeranampalayam Sivakumar, A. N., Deogun, J. S.,Scott,S.,Schnable,J.C.,&Shi,Y. (2021).MaizetasseldetectionfromUAVimageryusingDeepLearning. FrontiersinRoboticsandAl,8.

Andersen,J.R.,Schrag,T.,Melchinger,A.E.,Zein,I.,&Lübberstedt,T. (2005). Validation of DWARF8 polymorphisms associated with flowering time in elite Europeaninbredlinesofmaize (ZeamaysL.). TheoreticalandAppliedGenetics, 111 (2),206-217.

ANDERSON, S. L. & MURRAY, S. C. 2020. R/UAStools: : plotshpcreate: Createmulti-polygonshapefilesforextractionofresearchplotscaleagricultureremotesensingdata. Frontiers in plant science, 11, 511768.

Anderson, S. L., Murray, S. C., Malambo, L., Ratcliff, C., Popescu, S., Cope, D.,Chang, A., Jung, J., & Thomasson, J. A. (2019). Prediction of maize grain yield before maturity usingimprovedtemporalheightestimatesofunmannedaerialsystems. ThePlantPhenomeJournal,2 (1), 1-15.

Anderson, S.L., Murray, S.C., Chen, Y., Malambo, L., Chang, A., Popescu, S.,Cope,D.andJung,J.,” Unoccupiedaerialsystemenabledfunctionalmodelingofmaizeheightreveals dynamicexpressionofloci, “PlantDirect,4 (5),e00223. (2020).

AQUIL, M. A. I. & ISHAK, W. H. W. 2021. Evaluation of scratch and pre- trainedconvolutionalneuralnetworksfortheclassificationofTomatoplantdiseases.IAESInternationalJo urnalofArtificialIntelligence, 10,467.

Araus, J. L., & Cairns, J. E. (2014). Field high-throughput phenotyping: TheNewCropBreedingFrontier.TrendsinPlantScience, 19 (1),52-61.

Barzin, R., Pathak, R., Lotfi, H., Varco, J., & Bora, G. C. (2020). Use of UASmultispectral imageryatdifferentphysiologicalstagesforyieldpredictionandinputresourceoptimizationincorn.

### RemoteSensing,12 (15),2392.

BATES, D., MÄCHLER, M., BOLKER, B. & WALKER, S. 2014. Fitting linear mixed-effects models using Ime4. arXiv preprint arXiv: 1406.5823.

Baum, M. E., Archontoulis, S. V., & Licht, M. A. (2019). Planting date, hybridmaturity, and weather effects on maize yield and crop stage. Agronomy Journal, 111 (1), 303-313.

Bernardo, R. and Yu, J., “Prospects for genomewide selection for quantitativetraitsinmaize,” CropScience,47 (3),1082-1090 (2007).

Boland, M.R., Shahn, Z., Madigan, D., Hripcsak, G. and Tatonetti, N.P.,

“Birthmonthaffectslifetimediseaserisk: aphenome- widemethod,” JournaloftheAmericanMedicalInformaticsAssociation,22 (5), 1042-1053 (2015).

Box, J.F., “Guinness, Gosset, Fisher, and small samples,” Statistical science,45-52 (1987).

Brewer,K.,Clulow,A.,Sibanda,M.,Gokool,S.,Odindi,J.,Mutanga,O.,Naiken,V.,C himonyo, V.G.andMabhaudhi, T., “Estimationofmaizefoliartemperatureandstomatal conductance as indicators of water stress based on optical and thermal imagery acquiredusinganUnmannedAerialVehicle (UAV) platform,” Drones,6 (7),169 (2022).

Buckler, E.S.,Holland,J.B.,Bradbury,P.J.,Acharya,C.B.,Brown,P.J.,Browne, C., Ersoz, E., Flint-Garcia, S., Garcia, A., Glaubitz, J. C., Goodman, M. M., Harjes, C., Guill, K.,Kroon,D.E.,Larsson,S.,Lepak,N.K.,Li,H.,Mitchell,S.E.,Pressoir,G., . . . . McMullen,M. D. (2009). Thegeneticarchitectureofmaizefloweringtime.Science,325 (5941),714718.

CAI, J., OKAMOTO, M., ATIENO, J., SUTTON, T., LI, Y. & MIKLAVCIC, S. J. 2016. Quantifying the onset and progression of plant senescence by color image analysis for highthroughputapplications.PLOSOne, 11,e0157102.

Cárcova, J., Uribelarrea, M., Borrás, L., Otegui, M. E., & Westgate, M. E. (2000).

Synchronous pollination within and between ears improves kernel set in maize. Crop Science,40 (4),1056-1061.

Carroll,A.A.,Clarke,J.,Fahlgren,N.,Gehan,M.A.,Lawrence- Dill, C.J.andLorence, A., “NAPPN: Who we are, where we are going, and Why You Should Join Us!,” The PlantPhenomeJournal,2 (1), 1-4 (2019).

Chen,C.J.,Rutkoski,J.,Schnable,J.C.,Murray,S. C.,Wang,L.,Jin,X.,Stich,B., Crossa, J., Hayes, B. J., & Zhang, Z. (2022). Role of the genomics-phenomics-agronomy paradigminplantbreeding.PlantBreedingReviews, 627-673.

CHEN, K., ZHANG, X., PETERSEN, A. & MÜLLER, H.-G. 2017. Quantifying infinite dimensional data: Functional data analysis in action. Statistics in Biosciences, 9, 582-604.

Chen, R., Chu, T., Landivar, J.A., Yang, C. and Maeda, M.M., “Monitoringcotton (Gossypium hirsutum L.) germination using ultrahigh-resolution UAS images,” PrecisionAgriculture,19 (1),161177 (2018).

CHEN, Y. & DONG, H. 2016. Mechanisms and regulation of senescence andmaturityperformanceincotton.FieldCropsResearch, 189,1-9.

Chen, Y., “High-density linkage map construction, mapping of agronomictraits in tropical maize (Zea Mays L.) and validating SNPs controlling maize grain yield and plantheightinsouthernhybridtestcrosses” (TexasA&MDoctoraldissertation) 2016.

Chivasa, W., Mutanga, O., & Burgueño, J. (2021). UAV-based high- throughputphenotypingtoincreasepredictionandselectionaccuracyinmaizevarietiesunderartificialM SVinoculation.ComputersandElectronicsinAgriculture,184,106128.

Chu, T., Starek, M.J., Brewer, M.J., Murray, S.C. and Pruter, L.S., “Assessinglodgingseverityoveranexperimentalmaize (Zea maysL.) fieldusingUASimages,” RemoteSensing,9 (9),923 (2017).

Church, D.M., “A next-generation human genome sequence,”

### Science,376 (6588),34-35 (2022).

Currie, J. and Vogl, T., “Early-life health and adult circumstance in developingcountries,” Annu. Rev. Econ., 5 (1), 1-36 (2013). Boland, M.R., Shahn, Z., Madigan, D., Hripcsak,

G. and Tatonetti, N.P., “Birth month affects lifetime disease risk: a phenome-wide method,” JournaloftheAmericanMedicalInformaticsAssociation, 22 (5), 1042-1053 (2015).

Currie, J. and Vogl, T., “Early-life health and adult circumstance in developingcountries,” Annu.Rev.Econ.,5 (1),1-36 (2013).

Danilevicz, M. F., Bayer, P. E., Boussaid, F., Bennamoun, M., & Edwards, D. (2021).Maizeyieldpredictionatanearlydevelopmentalstageusingmultispectralimagesandgenot ypedataforpreliminaryhybridselection.RemoteSensing,13 (19),3976.

Das, A., Schneider, H., Burridge, J., Ascanio, A.K.M., Wojciechowski, T., Topp,C.N., Lynch, J.P., Weitz, J. S. and Bucksch, A., “Digital imaging of root traits (DIRT): a high- throughputcomputingand collaboration platform for field-basedroot phenomics, “Plantmethods, 11 (1), 1-12 (2015).

Daynard, T. B., Tanner, J. W., & Duncan, W. G. (1971). Duration of the grainfilling periodandits relationtograinyieldincorn,zeamaysl.CropScience,11 (1),45-48.

DEJOODE, D. R. & WENDEL, J. F. 1992. Genetic diversity and origin of theHawaiian Islands cotton, Gossypium tomentosum. AmericanJournal ofBotany, 79, 1311- 1319.

Delseny, M., Han, B., & Hsing, Y. I. (2010). High throughput DNA sequencing: Thenewsequencingrevolution.PlantScience, 179 (5),407-422.

DESALVIO, A. J. 2024. Supplementary Data-Temporal Image SandwichesEnableLinkbetweenFunctionalDataAnalysisandDeepLearningforSingle- PlantCottonSenescence [Online].

DESALVIO, A. J., ADAK, A., MURRAY, S. C., WILDE, S. C. & ISAKEIT, T. 2022.

Phenomic data-facilitated rust and senescence prediction in maize using machine learningalgorithms.Scientificreports, 12,1-14.

DeSalvio, A.J., Adak, A., Murray, S.C., Wilde, S.C. and Isakeit, T., “Phenomicdata- facilitatedrustandsenescencepredictioninmaizeusingmachinelearningalgorithms,” Scientificrepor ts, 12 (1), 114 (2022).

DONG, H., LI, W., TANG, W., LI, Z., ZHANG, D. & NIU, Y. 2006. Yield, quality and leaf senescence of cotton grown at varying planting dates and plant densities in the YellowRiverValleyofChina.FieldCropsResearch,98,106-115.

East, E.M., “The relation of certain biological principles to plant breeding, “ConnecticutAgriculturalExperimentStation, No.158, (1907).

ERHAN, D., BENGIO, Y., COURVILLE, A. & VINCENT, P. 2009. Visualizing higher-layer features of a deep network. University of Montreal, 1341, 1.

Ezenne, G.I., Jupp, L., Mantel, S. K. and Tanner, J.L., “Current and potentialcapabilitiesofUASforcropwaterproductivityinprecisionagriculture,” AgriculturalWaterM anagement,218,158-164 (2019).

FitriaWidiawati,I., Nugrahapraja,H.,&Fajriyah,R. (2018).K-NearestNeighbor (KNN) analysis on genes expression datasets of maize nested association mapping (NAM) showed confident classification on organ-specific expression. 2018 1st International Conference onBioinformatics, Biotechnology, andBiomedicalEngineering- BioinformaticsandBiomedicalEngineering.

Fu, Y., Wen, T.J., Ronin, Y.I., Chen, H.D., Guo, L., Mester, D.I., Yang, Y., Lee, M., Korol, A.B., Ashlock, D. A. and Schnable, P.S., “Genetic dissection of intermated recombinantinbredlinesusinganewgeneticmapofmaize,” Genetics, 174 (3),1671-1683 (2006).

FURBANK, R. T. & TESTER, M. 2011. Phenomics-technologies to relieve thephenotypingbottleneck.Trendsinplantscience, 16,635-644.

GAN, S. & AMASINO, R. M. 1997. Making sense of senescence (moleculargeneticregulationandmanipulationofleafsenescence).Plantphysiology,113,313.

GAN, S. 2003. Mitotic and postmitotic senescence in plants. Science of AgingKnowledgeEnvironment,2003,re7-re7.

Gano, B., Bhadra, S., Vilbig, J. M., Ahmed, N., Sagan, V., & Shakoor, N. (2024). Drone I based imaging sensors, techniques, and applications in plant phenotyping for cropbreeding: Acomprehensivereview. ThePlantPhenomeJournal,7 (1).

Gehan,M.A.,Fahlgren,N.,Abbasi,A.,Berry,J.C.,Callen,S.T.,Chavez,L.,Doust, A.N., Feldman, M.J., Gilbert, K.B., Hodge, J. G. and Hoyer, J.S., “PlantCV v2: Image analysissoftwareforhighthroughputplantphenotyping, “PeerJ,5,e4088 (2017). Georgiades, E., Klissouras, V., Baulch,J., Wang,G.andPitsiladis, Y.,” Whynature prevails over nurture in the making of the elite athlete,” BMC genomics, 18 (8), 59-66 (2017).

GHOSAL, S., BLYSTONE, D., SINGH, A. K., GANAPATHYSUBRAMANIAN, B., SINGH, A. & SARKAR, S. 2018. An explainable deep machine vision framework for plant stressphenotyping.ProceedingsoftheNationalAcademyofSciences, 115,4613-4618.

GLOROT, X., BORDES, A. & BENGIO, Y. Deep sparse rectifier neural networks. 20112011. JMLR Workshop and ConferenceProceedings, 315-323.

Goudet, J., Kay, T. and Weir, B.S., “How to estimate kinship,”

### Molecularecology,27 (20),4121-4135 (2018).

GREGERSEN, P. L. 2011. Senescence and nutrient remobilization in cropplants. Themolecularandphysiologicalbasisofnutrientuseefficiencyincrops, 83-102.

GREGERSEN, P. L., CULETIC, A., BOSCHIAN, L. & KRUPINSKA, K. 2013. Plant senescence and crop productivity. Plant molecular biology, 82, 603-622.

GUO, X., QIU, Y., NETTLETON, D. & SCHNABLE, P. S. 2023. High-Throughput Field Plant Phenotyping: A Self-Supervised Sequential CNN Method to Segment Overlapping Plants.PlantPhenomics,5,0052.

GUO, Y., LIU, Y., OERLEMANS, A., LAO, S., WU, S. & LEW, M. S. 2016. Deep learning for visual understanding: A review. Neurocomputing, 187, 27-48.

HAVÉ, M., MARMAGNE, A., CHARDON, F. & MASCLAUX-DAUBRESSE, C. 2017. Nitrogen remobilization during leaf senescence: lessons from Arabidopsis to crops. Journal ofExperimentalBotany,68,2513-2529.

Henkhaus, N., Bartlett, M., Gang, D., Grumet, R., Jordon-Thaden, I., Lorence,A.,Lyons,E.,Miller,S.,Murray,S.,Nelson,A.and Specht,C., “Plantsciencedecadalvision2020- 2030: Reimagining thepotential ofplants for a healthy andsustainable future,”

Plantdirect,4 (8),e00252 (2020).

Houle, D., Govindaraju, D. R. and Omholt, S., “Phenomics: the nextchallenge,” Naturereviewsgenetics, 11 (12),855-866 (2010).

HULSE-KEMP, A. M., ASHRAFI, H., ZHENG, X., WANG, F., HOEGENAUER, K.

A.,MAEDA, A. B. V., YANG, S. S., STOFFEL, K., MATVIENKO, M. & CLEMONS, K. 2014. Development and bin mapping of gene-associated interspecific SNPs for cotton (Gossypium hirsutum L.) introgressionbreedingefforts.BMCgenomics, 15,1-14.

Izzam, A. (2017). Genetic variability and correlation studies for morphologicalandyieldtraitsinmaize (ZeamaysL.).PureandAppliedBiology,6 (4).

Jez,J.M., Topp,C.N.,Matthews,M.L.andMarshall-Colón,A., “Multiscaleplant modeling: from genome to phenome and beyond.” Emerging topics in life sciences, 5 (2), 231- 237. (2021).

JUNG, M., SONG, J. S., SHIN, A.-Y., CHOI, B., GO, S., KWON, S.-Y., PARK, J., PARK, S. G. & KIM, Y.-M. 2023. Construction of deep learning-based disease detection model inplants.ScientificReports, 13,7331.

Karami, A., Quijano, K., & Crawford, M. (2021). Advancing Tassel Detectionandcounting: Annotationandalgorithms.RemoteSensing, 13 (15),2881.

KARHUNEN, K.1946. Zur spektraltheorie stochastischer prozesse. Ann.Acad. Sci. Fennicae, Al, 34.

Khan, S. U., Zheng, Y., Chachar, Z., Zhang, X., Zhou, G., Zong, N., Leng, P., &Zhao, J. (2022). Dissection of maize drought tolerance at the flowering stage using genome- wideassociationstudies.Genes, 13 (4),564.

KIM, H. J., PARK, J.-H., KIM, J., KIM, J. J., HONG, S., KIM, J., KIM, J. H., WOO, H. R.,HYEON,C.&LIM,P.O.2018.Time-evolvinggeneticnetworksreveala NACtroikathatnegatively regulates leaf senescence in Arabidopsis. Proceedings of the National Academy ofSciences,115,E4930-E4939.

KNYSHOV, A., HOANG, S. & WEIRAUCH, C. 2021. Pretrained convolutionalneural networks perform well in a challenging test case: identification of plant bugs

(Hemiptera: Miridae) usingasmallnumberoftrainingimages. InsectSystematicsandDiversity,5,3.

Krause, M.R., González-Pérez, L., Crossa, J., Pérez-Rodríguez, P., Montesinos- López,O.,Singh,R.P.,Dreisigacker,S.,Poland,J.,Rutkoski,J.,Sorrells,M.

andGore,M.A., “Hyperspectralreflectance- derivedrelationshipmatricesforgenomicpredictionofgrainyieldinwheat,” G3: Genes,Genomes,Geneti cs,9 (4),1231-1247 (2019).

Kumar,A.,Desai,S.V.,Balasubramanian, V.N.,Rajalakshmi,P.,Guo, W.,Balaji Naik, B., Balram, M., & Desai, U. B. (2021). Efficient maize tassel-detection method using UAVbasedremotesensing.RemoteSensingApplications: SocietyandEnvironment,23,100549.

Kumar, C., Mubvumba, P., Huang, Y., Dhillon, J., & Reddy, K. (2023). Multi- stagecornyieldpredictionusinghigh- resolutionUAVmultispectraldataandMachineLearningModels.Agronomy,13 (5),1277.

Kurtulmuş, F., & Kavdir, i. (2014). Detecting corn tassels using computervisionandsupportVectorMachines.ExpertSystemswithApplications,41 (16),7390- 7397.

Lachowiec, J., Feldman, M. J., Matias, F. I., Lebauer, D., & Gregory, A. (2024).

Unoccupied Aerial Systems Adoption inAgriculturalResearch.

Lander, E. S. “Initial impact of the sequencing of the human genome, “Nature,470 (7333),187-197 (2011).

Lane, H.M.,&Murray,S.C. (2021).Highthroughputcanproducebetterdecisions than high accuracy when phenotyping plant populations. Crop Science, 61 (5), 3301-3313. Lane, H.M., Murray, S.C., Montesinos López, O.A., Montesinos López,

A.,Crossa,J.,Rooney,D.K.,Barrero- Farfan,I.D.,DeLaFuente,G.N.andMorgan,C.L.,” Phenomicselectionandpredictionofmaizegrainyie Idfromnear- infraredreflectancespectroscopyofkernels, “ThePlantPhenomeJournal,3 (1),e20002 (2020).

Langstroff, A., Heuermann, M.C., Stahl, A. and Junker, A., “Opportunities andlimitsofcontrolled- environmentplantphenotypingforclimateresponsetraits,” TheoreticalandAppliedGenetics,1- 16 (2021).

LECUN, Y., BENGIO, Y. & HINTON, G. 2015. Deep learning. nature, 521, 436-

444.

LECUN, Y., BOTTOU, L., BENGIO, Y. & HAFFNER, P. 1998. Gradient-based learning applied to document recognition. Proceedings of the IEEE, 86, 2278-2324.

LEE, J., KANG, M. H., KIM, J. Y. & LIM, P. O. 2021. The role of light and circadian clock in regulation of leaf senescence. Frontiers in Plant Science, 12, 669170.

Lee, M., Sharopova, N., Beavis, W.D., Grant, D., Katt, M., Blair, D. andHallauer,A., “Expanding thegeneticmapofmaizewiththeintermatedB73xMo17 (IBM) population, “Plantmolecularbiol ogy,48 (5),453-461 (2002).

LIM, P. O., KIM, H. J. & GIL NAM, H. 2007. Leaf senescence. Annu. Rev.

### PlantBiol., 58,115136.

LIM, P. O., WOO, H. R. & NAM, H. G. 2003. Molecular genetics of leafsenescenceinArabidopsis.Trendsinplantscience,8,272-278.

LOÈVE, M. 1946. Fonctions aléatoires à décomposition orthogonaleexponentielle.LaRevueScientifique,84,159-162.

Lima,D.C.,Aviles,A.C.,Alpers,R.T.,Perkins,A.,Schoemaker,D.L.,Costa,M., Michel, K. J., Kaeppler, S., Ertl, D., Romay, M. C., Gage, J. L., Holland, J., Beissinger, T., Bohn, M., Buckler, E., Edwards, J., Flint-Garcia, S., Gore, M. A., Hirsch, C. N., . . . de Leon, N. (2023). 2020- 2021fieldseasonsofMaizeGxEprojectwithinthegenomestofieldsinitiative. BMCResearchNotes, 16 (1).

Liu, F., Van Der Lijn, F., Schurmann, C., Zhu, G., Chakravarty, M.M., Hysi, P.G.,Wollstein,A.,Lao,O.,DeBruijne,M.,Ikram,M.A.andVanDerLugt,A.,” A genome- wideassociationstudyidentifiesfivelociinfluencingfacialmorphologyinEuropeans, “PloSGeneticse100 2932 (2012).

Liu, Y., Cen, C., Che, Y., Ke, R., Ma, Y., & Ma, Y. (2020). Detection of maizetasselsfromUAVRGBimagerywithfasterR-CNN.RemoteSensing, 12 (2),338.

Lu, H., & Cao, Z. (2020). TASSELNETV2+: A fast implementation for high- throughputplantcountingfromhigh-resolutionRGBimagery.FrontiersinPlantScience,11.

Lu, H., Cao, Z., Xiao, Y., Zhuang, B., & Shen, C. (2017). TasselNet: Countingmaizetasselsinthewildvialocalcountsregressionnetwork.PlantMethods, 13 (1).

LYU, J. I. L., BAEK, S. H., JUNG, S., CHU, H., NAM, H. G., KIM, J. & LIM, P. O. 2017. High-throughput and computational study of leaf senescence through a phenomic approach.FrontiersinPlantScience,8,250.

Mace,E.S.,Hunt,C.H.,&Jordan,D.R. (2013).Supermodels: Sorghumandmaize provide mutual insight into the genetics of flowering time. Theoretical and Applied Genetics,126 (5) ,1377-1395.

MAKANZA, R., ZAMAN-ALLAH, M., CAIRNS, J. E., MAGOROKOSHO, C., TAREKEGNE, A., OLSEN, M. & PRASANNA, B. M. 2018. High-throughput phenotyping of canopy cover and senescence in maize field trials using aerial digital canopy imaging. Remote Sensing, 10,330.

Malouff, J.M., Rooke, S. E. and Schutte, N.S., “The heritability of humanbehavior: Results of aggregating meta-analyses,” Current Psychology, 27 (3), 153-161 (2008). Marks, J., “Historiography of eugenics,” American Journal of

HumanGenetics,52 (3),650 (1993). Marshall-

Colon,A.,Long,S.P.,Allen,D.K.,Allen,G.,Beard,D.A.,Benes,B.,VonCaemmerer,S.,Christensen,A.J.,Cox, D.J., Hart,J.C.andHirst,P.M., “Cropsinsilico: generating virtual crops using an integrative and multi- scale modeling platform,” Frontiers in plantscience,8,786 (2017).

Matias, F. I., Caraza Harter, M. V., & Endelman, J. B. (2020).

Fieldimager: AnRpackagetoanalyzeorthomosaicimagesfromagriculturalfieldtrials. ThePlantPheno meJournal,3 (1).

Matias, F. I., Green, A., Lachowiec, J. A., LeBauer, D., & Feldman, M., “Bison- Fly: An open-source UAV pipeline for plant breeding data collection,” The Plant Phenome Journal,5 (1),e20048 (2022).

MINERVINI, M., ABDELSAMEA, M. M. & TSAFTARIS, S. A. 2014. Image-based plant phenotyping with incremental learning and active contours. Ecological Informatics, 23, 35 -48.

Minervini, M.,Scharr, H.,&Tsaftaris,S.A. (2015).Imageanalysis: Thenewbottlene ck in plant phenotyping [applications corner]. IEEE Signal Processing Magazine, 32 (4), 126-131.

Mirnezami,S.V.,Srinivasan,S.,Zhou,Y.,Schnable,P.S.,&Ganapathysubramani an, B. (2021). Detection of the progression of anthesis in field-grown maizetassels: Acasestudy.PlantPhenomics, 2021.

MOHANTY, S. P., HUGHES, D. P. & SALATHÉ, M. 2016. Using deep learning for imagebased plant disease detection. Frontiers in plant science, 7, 1419.

MORRIS, J. S. 2015. Functional regression. Annual Review of Statistics and

### ItsApplication,2,321-359.

Murcia, H. F., Tilaguy, S., & Ouazaa, S. (2021). Development of a low- costsystemfor3DOrchardMappingIntegratingUGVandLidar.Plants,10 (12),2804.

MURRAY, S. C., ADAK, A., DESALVIO, A. & LANE, H. 2022. Temporal field phenomics allows discovery of nature AND nurture, so can we saturate the phenome? AuthoreaPreprints.

Neupane, K.andBaysal-Gurel, F., “Automaticidentificationandmonitoringof plant diseases using unmanned aerial vehicles: A review,” Remote Sensing, 13 (19), 3841 (2021).

NIGUS, E. A., TAYE, G. B., GIRMAW, D. W. & SALAU, A. O. 2023. Development of a Model for Detection and Grading of Stem Rust in Wheat Using Deep Learning. MultimediaToolsandApplications, 1-28.

NIU, H., PEDDAGUDREDDYGARI, J. R., BHANDARI, M., LANDIVAR, J. A., BEDNARZ,C.W.&DUFFIELD,N.2024. In-SeasonCottonYieldPredictionwithScale-AwareConvolutional Neural Network Models and Unmanned Aerial Vehicle RGB Imagery. Sensors, 24,2432.

NIU, Y. H., DONG, H. Z., LI, W.-J. & LI, H.-M. 2007. Effects of removal of earlyfruiting branches on yield, fiber quality and premature senescence in Bt transgenic cotton. CottonSci,19,52-56.

NSF. 2010 Project. “To determine the function of all genes in Arabidopsisthalianabytheyear2010” (21October 2010).

OUGHAM, H., HÖRTENSTEINER, S., ARMSTEAD, I., DONNISON, I., KING, I., THOMAS, H. & MUR, L. 2008. The control of chlorophyll catabolismand the status of yellowing as abiomarkerofleafsenescence.PlantBiology,10,4-14.

Panofsky, A., Dasgupta, K. and Iturriaga, N., “How White nationalists mobilizegenetics: Fromgeneticancestryandhumanbiodiversitytocounterscienceandmetapolitics, “A mericanJournalofPhysicalAnthropology,175 (2),387-398. (2021).

Pauli, D., Andrade-Sanchez, P., Carmo-Silva, A.E., Gazave, E., French, A.N.,Heun,J.,Hunsaker,D.J.,Lipka,A.E.,Setter,T.L.,Strand,R.J.andThorp,K.R.,” Field-basedhigh- throughput plant phenotyping reveals the temporal patterns of quantitative trait loci associatedwithstress-responsivetraitsincotton, “G3: Genes,Genomes,Genetics,6 (4),865- 879 (2016).

PAWARA, P., OKAFOR, E., SURINTA, O., SCHOMAKER, L. & WIERING, M.

Comparing local descriptors and bags of visual words to deep convolutional neural networks forplantrecognition.20172017.ICPRAM.

Poland, J. A., & Rife, T. W. (2012). Genotyping] by sequencing for PlantBreedingandGenetics. ThePlantGenome,5 (3).

Pugh, N. A., Horne, D. W., Murray, S. C., Carvalho, G., Malambo, L., Jung, J.,Chang, A., Maeda, M., Popescu, S., Chu, T., Starek, M. J., Brewer, M. J., Richardson, G., & Rooney,

W. L. (2018). Temporal estimates of crop growth in sorghum and maize breeding enabled byunmannedaerialsystems. ThePlantPhenomeJournal,1 (1),1-10.

RattalinoEdreira,J.I., BudakliCarpici, E.,Sammarro,D.,&Otegui,M.E. (2011). Heat stress effects around flowering on kernel set of temperate and tropical maize hybrids.FieldCropsResearch, 123 (2),62-73.

Ren, S., He, K., Girshick, R., & Sun, J. (2015). Faster R-CNN: Towards real- timeobjectdetectionwithregionproposalnetworks.IEEETransactionsonPatternAnalysisandMachinel ntelligence,39 (6),1137-1149.

Rexroad, C., Vallet, J., Matukumalli, L.K., Reecy, J., Bickhart, D., Blackburn, H.,Boggess,M.,Cheng,H.,Clutter,A.,Cockett,N.andErnst,C.,” Genometophenome: improvinganimal health, production, and well-being-a new USDA blueprint for animal genome research 2018- 2027, “Frontiersingenetics,10,327 (2019).

RIBERA, J., CHEN, Y., BOOMSMA, C. & DELP, E. J. Counting plants using deeplearning.20172017.IEEE,1344-1348.

Richmond, S., Howe, L.J., Lewis, S., Stergiakouli, E. and Zhurov, A., “Facialgenetics: abriefoverview, “Frontiersingenetics,9,462 (2018).

Rincent,R.,Charpentier,J.P.,Faivre-Rampant,P.,Paux,E.,LeGouis,J.,Bastien, C. and Segura, V., “Phenomic selection is a low-cost and high-throughput method based onindirect predictions: proof of concept on wheat and poplar,” G3: Genes, Genomes, Genetics, 8 (12), 3961- 3972 (2018).

Robert, P., Brault, C., Rincent, R. and Segura, V., “Phenomic selection: A newandefficientalternative togenomicselection,” In [ComplexTraitPrediction] Humana, NewYork,397-420 (2022).

Rodene,E.,Fernando,G.D.,Piyush,V.,Ge,Y.,Schnable,J.C.,Ghosh,S.,&Yang, J. (2024). Image filtering to improve maize tassel detection accuracy using machine learningalgorithms.Sensors,24 (7),2172.

SAHA, S., RASKA, D. A. & STELLY, D. M. 2006. Upland Cotton (Gossypium hirsutum L.) x Hawaiian Cotton (G. tomentosum Nutt. Ex. Seem.) F1 hybrid hypoaneuploidchromosomesubstitutionseries.

Seethepalli, A., Dhakal, K., Griffiths, M., Guo, H., Freschet, G. T. and York, L.M., “RhizoVisionExplorer: open- sourcesoftwareforrootimageanalysisandmeasurementstandardization,” AoBplants,13 (6), plab0 56 (2021).

Shakoor, N., Northrup, D., Murray, S., & Mockler, T. C. “Big data drivenagriculture: bigdataanalyticsinplantbreeding,genomics, andtheuseofremotesensingtech nologiestoadvancecropproductivity,” ThePlantPhenomeJournal,2 (1),1-8. (2019).

Shao, M., Nie, C., Cheng, M., Yu, X., Bai, Y., Ming, B., Song, H., & Jin, X. (2021). Quantifying effect of tassels on near-ground maize canopy RGB images using deep learningsegmentationalgorithm.PrecisionAgriculture,23 (2),400-418.

Shao, M., Nie, C., Zhang, A., Shi, L., Zha, Y., Xu, H., Yang, H., Yu, X., Bai, Y., Liu, S., Cheng, M., Lin, T., Cui, N., Wu, W., & Jin, X. (2023). Quantifying effect of maize tassels on LAlestimationbasedonmultispectralimageryandmachinelearningmethods.ComputersandElectronics inAgriculture,211,108029.

Shi, Y., Thomasson, J.A., Murray, S.C., Pugh, N.A., Rooney, W.L., Shafian, S.,Rajan,N.,Rouze,G.,Morgan,C.L.,Neely,H.L.and Rana,A., “Unmannedaerialvehicles forhigh- throughputphenotypingandagronomicresearch, “PloSone,11 (7),e0159781 (2016).

SHIM, J., MANGAT, P. K. & ANGELES-SHIM, R. B. 2018. Natural variation in wild Gossypium species as a tool to broaden the genetic base of cultivated cotton. J. Plant Sci. Curr.Res,2.

SHRIVASTAVA, V. K., PRADHAN, M. K. & THAKUR, M. P. Application of pre- trained deep convolutional neural networks for rice plant disease classification. 2021 2021. IEEE,1023-1030.

SIMONYAN, K., VEDALDI, A. & ZISSERMAN, A. 2013. Deep inside convolutional networks: Visualising image classification models and saliency maps. arXiv preprintarXiv: 1312.6034.

Song,P.,Wang,J.,Guo,X.,Yang,W.,&Zhao,C. (2021).High- throughputphenotyping: Breaking through the bottleneck in future crop breeding. The Crop Journal, 9 (3), 633-645.

Speed, D. and Balding, D.J., “Relatedness in the post-genomic era: is it stilluseful?,” NatureReviewsGenetics, 16 (1),33-44 (2015).

SRIVASTAVA, N., HINTON, G., KRIZHEVSKY, A., SUTSKEVER, I. & SALAKHUTDINOV, R. 2014. Dropout: a simple way to prevent neural networks from overfitting. Thejournalofmachinelearningresearch, 15,1929-1958.

Sunoj, S., Cho, J., Guinness, J., van Aardt, J., Czymmek, K. J., & Ketterings, Q. M. (2021). Corn grain yield prediction and mapping from unmanned aerial system (UAS) multispectralimagery.RemoteSensing, 13 (19),3948.

Tirado, S. B., Hirsch, C. N., & Springer, N. M. (2020). UAV I based imagingplatformformonitoringmaizegrowththroughoutdevelopment.PlantDirect,4 (6).

TUCKER, C. J. 1979. Red and photographic infrared linear combinations formonitoringvegetation.RemotesensingofEnvironment,8,127-150.

### Tuggle,C.K.,Clarke,J.,Dekkers,J.,Ertl,D.,Lawrence-

Dill,C.J.,Lyons,E.,Murdoch, B.M., Scott, N.M. and Schnable, P.S., “The Agricultural Genome to Phenome

Initiative (AG2PI): creatingasharedvisionacrosscropandlivestockresearchcommunities, “Genomeb iology,23 (1),1-11 (2022).

UBBENS, J. R. & STAVNESS, I. 2017. Deep plant phenomics: a deep learningplatformforcomplexplantphenotypingtasks.Frontiersinplantscience,8,1190.

Ubbens,J.,Cieslak,M.,Prusinkiewicz,P.andStavness,l.,” Theuseofplantmodels in deep learning: an application to leaf counting in rosette plants,” Plant methods, 14 (1), 1- 10 (2018).

Ubbens, J. R. and Stavness, I., “Deep plant phenomics: a deep learningplatform for complex plantphenotypingtasks,” Frontiersinplantscience,8,1190 (2017).

WANG, J.-L., CHIOU, J.-M. & MÜLLER, H.-G. 2016. Functional data analysis. Annual Review of Statistics and its application, 3, 257-295.

Warrington,I.J.,&Kanemasu,E.T. (1983).Corngrowthresponsetotemperatur e and photoperiod I. Seedling emergence, tassel initiation, and anthesis1. AgronomyJournal, 75 (5),749-754.

Watt,M.,Fiorani,F.,Usadel,B.,Rascher,U.,Muller,O.andSchurr,U., “Phenotypi ng: new windows into the plant for breeders,” Annual review of plant biology, 71 689-712 (2020).

Wee, C. W., & Dinneny, J. R., “Tools for high-spatial and temporal- resolutionanalysisofenvironmentalresponsesinplants,” Biotechnologyletters,32 (10),1361- 1371 (2010).

WOEBBECKE, D. M., MEYER, G. E., VON BARGEN, K. & MORTENSEN, D. A. 1995. Color indices for weed identification under various soil, residue, and lighting conditions.TransactionsoftheASAE,38,259-269.

WOO, H. R., KIM, H. J., LIM, P. O. & NAM, H. G. 2019. Leaf senescence:
systems and dynamics aspects. Annual review of plant biology, 70, 347-376.

WOO, H. R., KOO, H. J., KIM, J., JEONG, H., YANG, J. O., LEE, I. H., JUN, J. H., CHOI, S. H., PARK, S. J. & KANG, B. 2016. Programming of plant leaf senescence with temporal andinter-organellarcoordinationoftranscriptomeinArabidopsis.Plantphysiology,171,452-467.

Worku,M.,Makumbi,D.,Beyene,Y.,Das,B.,Mugo,S.,Pixley,K.,Bänziger,M., Owino, F., Olsen, M., Asea, G., & Prasanna, B. M. (2016). Grain yield performance and floweringsynchronyofCimmyt′stropicalmaize (ZeamaysL.) parentalinbredlinesandsinglecrosses. Euphytica, 211 (3), 395-409.

Wu,D.,Li,X.,Tanaka,R.,Wood,J.C.,Tibbs-Cortes,L.E.,Magallanes- Lundback,M.,Bornowski,N.,Hamilton,J.P.,Vaillancourt,B.,Diepenbrock,C.H.andLi,X., “Combining GWAS and TWAS to identify candidate causal genes for tocochromanol levels in maizegrain,” bioRxiv (2022).

Wu, G., Miller, N. D., de Leon, N., Kaeppler, S. M., & Spalding, E. P. (2019). Predicting Zea mays flowering time, yield, and kernel dimensions by analyzing aerial images.FrontiersinPlantScience,10.

Wu,H.,Wiesner-Hanks,T.,Stewart,E.L.,DeChant,C.,Kaczmar,N.,Gore,M.A., Nelson, R. J. and Lipson, H.,. “Autonomous detection of plant disease symptoms directly fromaerialimagery,” Theplantphenomejournal,2 (1), pp. 1-9 (2019).

XIE, X., GE, Y., WALIA, H., YANG, J. & YU, H. 2023. Leaf-counting in monocotplantsusingdeepregressionmodels.Sensors,23,1890.

YOO, H.-J. 2015. Deep convolution neural networks in computer vision:
areview.IEIETransactionsonSmartProcessing&Computing,4,35-43.

YOSHIDA, Y. 1962. Nuclear control of chloroplast activity in Elodea leaf cells. Protoplasma, 54,476-492.

Yu,J.,Zhang,Z.,Zhu,C.,Tabanao,D.A.,Pressoir,G.,Tuinstra,M.R.,Kresovich, S., Todhunter, R. J. and Buckler, E.S., “Simulation appraisal of the adequacy of number ofbackgroundmarkersforrelationshipestimationinassociationmapping,” ThePlantGenome,2 (1) (2009 1.

Yu, X., Yin, D., Nie, C., Ming, B., Xu, H., Liu, Y., Bai, Y., Shao, M., Cheng, M., Liu, Y., Liu, S., Wang, Z., Wang, S., Shi, L., & Jin, X. (2022). Maize tassel area dynamic monitoringbasedonnear-groundandUAVRGBimagesbyU- Netmodel.ComputersandElectronicsinAgriculture, 203,107477.

Zan, X., Zhang, X., Xing, Z., Liu, W., Zhang, X., Su, W., Liu, Z., Zhao, Y., & Li, S. (2020).AutomaticdetectionofmaizetasselsfromUAVimagesbycombiningrandomforestclassifiera ndVGG16.RemoteSensing,12 (18),3049.

Zhang, L., Niu, Y., Zhang, H., Han, W., Li, G., Tang, J. and Peng, X., “Maizecanopy temperature extracted from UAV thermal and RGB imagery and its application in waterstressmonitoring, “Frontiersinplantscience,10,1270 (2019).

Zhang, W., Wu, S., Wen, W., Lu, X., Wang, C., Gou, W., Li, Y., Guo, X., & Zhao, C. (2023). Three-dimensional branch segmentation and phenotype extraction of Maize TasselbasedonDeepLearning.PlantMethods, 19 (1).

ZHANG, Z., RONG, J., WAGHMARE, V. N., CHEE, P. W., MAY, O. L., WRIGHT, R. J., GANNAWAY, J. R. & PATERSON, A. H. 2011. QTL alleles for improved fiber quality from a wildHawaiian cotton, Gossypium tomentosum. Theoretical and applied genetics, 123,1075- 1088.

ZHAO, Y., CHAN, Z., GAO, J., XING, L., CAO, M., YU, C., HU, Y., YOU, J., SHI, H. & ZHU, Y. 2016. ABA receptor PYL9 promotes drought resistance and leaf senescence. ProceedingsoftheNationalAcademyofSciences,113,1949-1954.

Zhu, X., Leiser, W. L., Hahn, V., & Würschum, T. (2021). Phenomic selection iscompetitivewithgenomicselectionforbreedingofcomplextraits. ThePlantPhenomeJournal,4 (1).

ZINGARETTI, L. M., GEZAN, S. A., FERRÃO, L. F. V., OSORIO, L. F., MONFORT, A., MUÑOZ, P. R., WHITAKER, V. M. & PÉREZ-ENCISO, M. 2020. Exploring deep learning for complextraitgenomicpredictioninpolyploidoutcrossingspecies.Frontiersinplantscience,11,506702.

1. A method of selecting biomarkers, the method comprising: generatingapluralityofbiomarkers;
selecting a subset of biomarkers from the plurality of biomarkers; and
determining a relationship between the subset of biomarkers and a phenotype of interest.

2. The method of claim 1, wherein selecting the subset of biomarkers comprisesselectingheritableorrepeatablesetofbiomarkersfromthepluralityofbiomarkers.

3. The method of claim 1, wherein the plurality of biomarkers comprises at least one ofgeneticdata, metabolomicdataorproteomicdata.

4. The method of claim 1, wherein the plurality of biomarkers are generated using aconvolutionalneuralnetwork.

5. Themethodofclaim1, whereinselectingthesubsetofbiomarkerscomprisesselecting biomarkers based a correlation between a biomarker of the plurality of biomarkers with adifferentbiomarkerofthepluralityofbiomarkers.

6. Themethodofclaim1, whereindeterminingarelationshipbetweenthesubsetofbiomark ers and the phenotype of interest is based on a machine learning test of prediction ability.

7. The method of claim 6, wherein the machine learning test of prediction abilitycomprisesalassotest, aridgeregressiontest, aBayesBorarandomforestregressiontest.

8. The method of claim 7, wherein the machine learning test further comprises crossvalidation.

9. The method of claim 1, wherein the plurality of biomarkers comprise temporally (longitudinally) measuredbiomarkers.

10. Themethodofclaim1, whereinthemethodfurthercomprisesdetermininganoptimal management strategy for improving health, production or other value added trait for asubject, wherein the optimal managementstrategy for thesubject is configured to change thephenotypeofinterestofthesubject.

11. The method of claim 1, wherein phenotype of interest comprises a disease or risk ofdisease.

12. The method of claim 1, wherein the phenotype comprises a plant phenotype.

13. The method of claim 1, wherein the plurality of biomarkers comprise image data.

14. The method of claim 13, further comprising decomposing the image data into apluralityofimagefeatures.

15. The method of claim 14, wherein the plurality of image features comprise estimatesofspectralreflectance,apositionororientationofasubject.

16. The method of claim 14, wherein the plurality of image features comprise estimatesofasizeofasubject.

17. A method of predicting a phenotype of interest of a subject, the method comprising: generatingapluralityofbiomarkers;
selecting a subset of biomarkers from the plurality of biomarkers; and
determining a relationship between the subset of biomarkers and the phenotype ofinterest;
receiving sample biomarkers; wherein the sample biomarkers comprise one or moremeasurementsofthesubject;and predicting the phenotype of interest of the subject based on the sample biomarkers and therelationshipbetweenthesubsetofbiomarkersandthephenotypeofinterest.

18. The method of claim 20, wherein selecting the subset of biomarkers comprisesselectingheritablebiomarkersfromthepluralityofbiomarkers.

19. The method of claim 20, wherein the plurality of biomarkers comprises at least oneofgeneticdata, metabolomicdataorproteomicdata.

20. The method of claim 20, wherein the plurality of biomarkers are generated using aneuralnetwork.
